(19) United States
(12) Patent Application Publication
(10) Pub. No.: US 2025/0381198 A1
(43) Pub. Date: Dec. 18, 2025

(54) MIDAZOLAM AND KETAMINE FOR ENHANCED SEDATION

(71) Applicant: Melt Pharmaceuticals, Inc., Brentwood, TN (US)

(72) Inventor: Larry Dillaha, Brentwood, TN (US)

(21) Appl. No.: 19/242,284

(22) Filed: Jun. 18, 2025

Related U.S. Application Data
(60) Provisional application No. 63/661,385, filed on Jun. 18, 2024.

(51) Int. Cl. A61K 31/5517; A61K 31/135; A61P 25/20

(52) U.S. Cl. CPC A61K 31/5517; A61K 31/135; A61P 25/20

(57) ABSTRACT

Provided herein are pharmaceutical compositions comprising midazolam and ketamine, and methods of inducing sedation (e.g., procedural sedation) in a subject using administration of such compositions, the compositions optionally including a pharmaceutically active compound of a third class. Compositions may be in sublingual or buccal form, or incorporated into vehicles for extended release. Methods for fabricating the compositions and using them for anesthesiological applications are also described.

### CROSS-REFERENCE TO RELATED APPLICATIONS

This application claims priority to U.S. Application No. 63/661,385, filed Jun. 18, 2024, the entire contents of which are incorporated by reference herein.

### FIELD OF THE TECHNOLOGY

The present invention relates generally to the field of pharmacology and more specifically to compositions comprising midazolam and ketamine, which have anesthetic properties that are useful in various kinds of medical practice, such as surgery or a medical procedure, and to methods of preparing and using such compositions (e.g., for sedation).

### BACKGROUND

The present disclosure relates to pharmaceutical formulations comprising a combination of midazolam and ketamine, and optionally one or more pharmaceutically active compounds of third class (e.g., an anesthetic, antiemetics, antianxiety medications and/or analgesics), and methods for using the same for providing anesthesia (e.g., inducing sedation) by administering such compositions orally, including such administrations as sublingual or buccal. The formulations may also include slow release reversal agents that would counteract the initial anesthesia effect.

It is necessary in many cases to use local anesthesia in the course of various surgical procedures, e.g., ophthalmic surgeries or urological interventions. For instance, when local anesthesia is employed during or prior to intraocular operations (e.g., topically applied local anesthesia), the occurrences of pain, anxiety, peri-operative stress, nausea, agitation, vomiting and the like are less frequent, which will typically have a very beneficial effect on the surgical experience and reducing the number of intraocular complications such as bleeding, secretions, cardiac and/or pulmonary complications, etc. The severity of those complications when they do occur will also be less pronounced when local anesthesia is used.

Traditionally, an intravenous route is used to administer medications. Alternatives to intravenous methods and therapies have been suggested and previously used for the treatment. In particular, oral administration of benzodiazepines, opioid analgesics, propofol, ketamine or etomidate utilizing the MAC procedure (monitored anesthesia care) has been suggested and tried, but no more than minimal to moderate improvement has been achieved by such methods. Therefore, there remains a need for better treatments of these disorders.

This patent specification discloses such pharmaceutical compositions suitable for anesthesiological applications (e.g., procedural sedation) that can achieve positive patient outcomes while free of drawbacks and deficiencies of existing methods and formulations. Methods of fabricating and administering the same are also discussed.

### SUMMARY OF THE INVENTION

In one aspect, provided herein is a method of inducing procedural sedation in a subject, the method comprising administering (e.g., sublingually administering) a pharmaceutical composition comprising midazolam and ketamine, wherein the administration achieves a level of sedation for procedural sedation in the subject. The time period of sedation may last for a time period of 1 hour or less (e.g., 45 minutes or less, 30 minutes or less, or 15 minutes or less).

In another aspect, provided herein is a method of inducing procedural sedation in a subject, the method comprising administering (e.g., sublingually administering) to the subject a first dose of a pharmaceutical composition comprising midazolam and ketamine; and administering (e.g., sublingually administering) to the subject a second dose of the pharmaceutical composition after the sublingual administration of the first dose, wherein the administration achieves a level of sedation for procedural sedation in the subject. The time period of sedation may last for a time period of 1 hour or less (e.g., 45 minutes or less, 30 minutes or less, or 15 minutes or less). The sublingually administration of the second dose of the pharmaceutical composition may occur within 30 minutes (e.g., within 15 minutes) after administering the first dose of the pharmaceutical composition.

In another aspect, provided herein is a method of reducing the occurrence of rescue, the method comprising administering (e.g., sublingually administering) a pharmaceutical composition comprising midazolam and ketamine to achieve procedural sedation in a subject. The rescue may be performed when the subject's level of sedation is a 1 on the Ramsay sedation scale. The rescue may be pre-operative or intra-operative. The occurrence of rescue may be reduced as compared to administration of midazolam alone or ketamine alone.

In such methods, the level of sedation achieved in the subject may be measured via the Ramsay sedation scale. The level of sedation achieved may be greater than achieved by administering midazolam alone, or ketamine alone.

In such methods, the weight ratio of midazolam to ketamine in the pharmaceutical composition may be about 1:5 to about 1:20, optionally about 3:25 (e.g. about 3 mg of midazolam and about 25 mg of ketamine) or about 3:50 (e.g., about 3 mg of midazolam and about 50 mg of ketamine).

Such methods may result in a Cmax of midazolam in the subject that is at least about 10% lower, at least about 20% lower, at least about 30% lower, at least about 40% lower, at least about 50% lower, at least about 60% lower, at least about 70% lower, or at least about 80% lower than a Cmax of midazolam resulting from intravenous administration of an equivalent amount of midazolam (e.g., less than or equal to about 140 ng/ml). Such methods may also result in an AUC0-t of midazolam in the subject that is not statistically different from or at least about 10% lower, at least about 20% lower, about least about 30% lower, at least about 40% lower, or at least about 50% lower than an AUC0-t of midazolam resulting from intravenous administration of an equivalent amount of midazolam. Such methods may also result in an AUCinf of midazolam in the subject that is not statistically different from or at least about 10% lower, at least about 20% lower, at least about 30% lower, at least about 40% lower, or at least about 50% lower than an AUCinf of midazolam resulting from intravenous administration of an equivalent amount of midazolam.

Such methods may result in a Cmax of 1 hydroxymidazolam in the subject that is at least about 25% greater, at least about 50% greater, at least about 100% greater, or at least about 200% greater than a Cmax of 1 hydroxymidazolam resulting from intravenous administration of an equal amount of midazolam (e.g., greater than or equal to about 6 ng/mL).

Such methods may result in a Cmax of ketamine in the subject that is at least about 10% lower, at least about 20% lower, at least about 30% lower, at least about 40% lower, at least about 50% lower, at least about 60% lower, at least about 70% lower, or at least about 80% lower than a Cmax achieved from intravenous administration of an equal amount of ketamine (e.g., less than or equal to about 275 ng/mL). Such methods may also result in an AUC0-t of ketamine in the subject that is at least about 10% lower, at least about 20% lower, at least about 30% lower, at least 40% about lower, at least about 50% lower, at least about 60% lower, or at least about 70% lower than an AUC0-t of ketamine resulting from intravenous administration of an equivalent amount of ketamine. Such methods may also result in an AUCinf of ketamine in the subject that is at least about 10% lower, at least about 20% lower, at least about 30% lower, at least about 40% lower, at least about 50% lower, at least about 60% lower, or at least about 70% lower than an AUCinf of ketamine resulting from intravenous administration of an equivalent amount of ketamine. Such methods may also result in a half-life (t1/2) of ketamine in the subject that is at least 1 hr, 2 hr, 3 hr, 4 hr, or 5 hr shorter than a half-life of ketamine resulting from intravenous administration of an equivalent amount of ketamine.

Such methods may result in a Cmax of norketamine in the subject that is at least about 10% greater, at least about 25% greater, at least about 50% greater, at least about 100% greater, at least about 150% greater, at least about 200% greater, at least about 300% greater, or at least about 400% greater than a Cmax of norketamine achieved from intravenous administration of an equal amount of ketamine (e.g., greater than or equal to about 30 ng/mL).

In another aspect, provided herein is a solid pharmaceutical composition formulated for sublingual and or buccal administration comprising about 3 mg of midazolam and about 50 mg of ketamine. The solid pharmaceutical composition may further comprise a third pharmaceutically active compound selected from a benzodiazepine receptor antagonist, non-benzodiazepine based sedative, β-blocker, α-2-adrenergic agonist, pain reliever, antiemetic medicament, non-steroid anti-inflammatory drug (NSAID), or antihistamine medicament, or a combination thereof or pharmaceutically acceptable salts, hydrates, solvates or N-oxides thereof. The solid pharmaceutical composition may be in a solid dosage form selected from a troche, lozenge, capsule, pill, cap, or bolus. The solid pharmaceutical composition may further comprise a binder, optionally selected from a binder as described herein. The solid pharmaceutical composition may further comprise an excipient, optionally selected from an excipient as described herein. Said solid pharmaceutical composition may be used in a method of inducing sedation in a subject (e.g. as described herein).

### BRIEF DESCRIPTION OF THE DRAWINGS

FIG. 1 shows mean plasma concentration-time profiles of the analyte midazolam on the linear scale, from the pharmacokinetics population after administration of MELT-100 midazolam and ketamine formulations, and midazolam IV alone. Open circles depict Treatment A (Test 1)=A single MELT-100 sublingual tablet (3 mg midazolam and 25 mg ketamine). Open triangles depict Treatment B (Test 2)=A single MELT-100 sublingual tablet (2×3 mg midazolam and 25 mg ketamine administered sublingually, which 15 min apart, total dose=6/50 mg). Open squares depict Treatment C (Reference 1)=A single dose of midazolam 3.5 mg administered as IV in 3 increments over 2 minutes of 1.5 mg, 1 mg, and 1 mg (2 minutes between each administration)

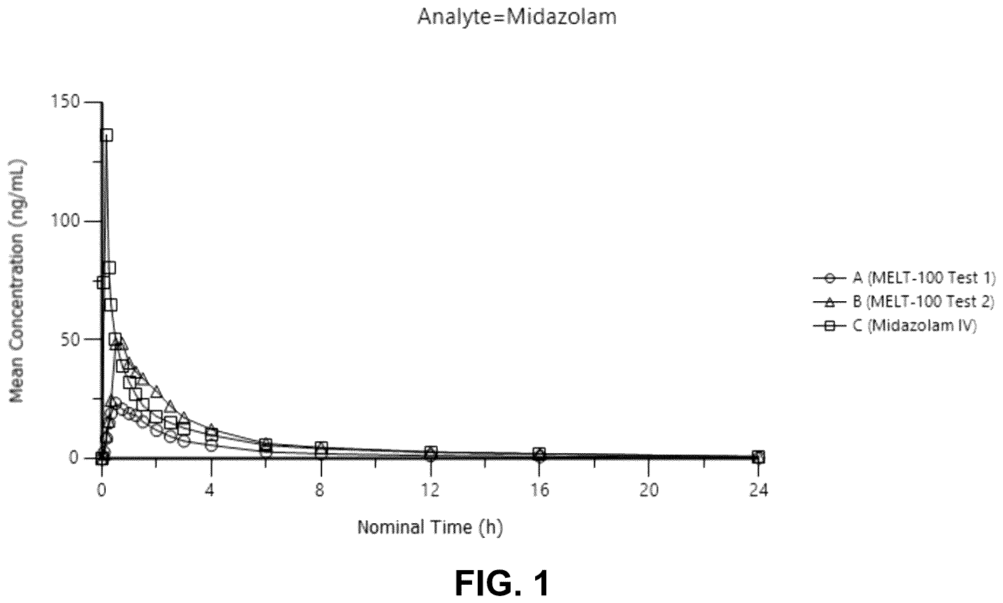

FIG. 2 shows mean plasma concentration-time profiles of the analyte 1-hydroxymidazolam on the linear scale, from the pharmacokinetics population after administration of MELT-100 midazolam and ketamine formulations, and midazolam IV alone. Open circles depict Treatment A (Test 1)=A single MELT-100 sublingual tablet (3 mg midazolam and 25 mg ketamine). Open triangles depict Treatment B (Test 2)=A single MELT-100 sublingual tablet (2×3 mg midazolam and 25 mg ketamine administered sublingually 15 min apart, total dose=6/50 mg). Open squares depict Treatment C (Reference 1)=A single dose of midazolam 3.5 mg administered as IV in 3 increments over 2 minutes of 1.5 mg, 1 mg, and 1 mg (2 minutes between each administration).

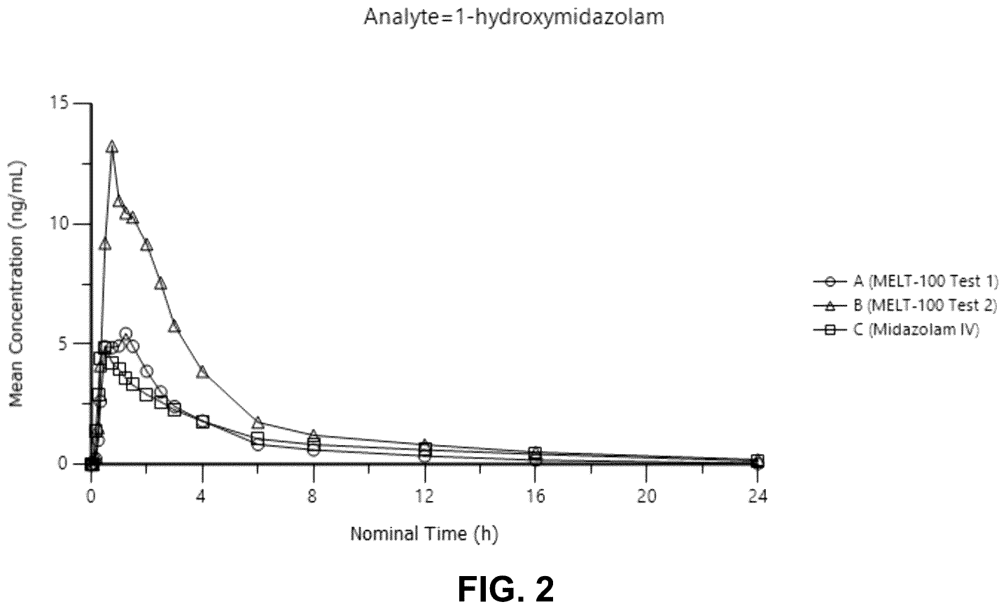

FIG. 3 shows mean plasma concentration-time profiles of the analyte ketamine on the linear scale, from the pharmacokinetics population after administration of MELT-100 midazolam and ketamine formulations, and ketamine IV alone. Open circles depict Treatment A (Test 1)=A single MELT-100 sublingual tablet (3 mg midazolam and 25 mg ketamine). Open triangles depict Treatment B (Test 2)=A single MELT-100 sublingual tablet (2×3 mg midazolam and 25 mg ketamine administered sublingually 15 min apart, total dose=6/50 mg). Open triangles depict Treatment D (Reference 2)=A single dose of ketamine 18 mg (10 mg/mL in a 20 mL vial) administered as IV over 5 minutes.

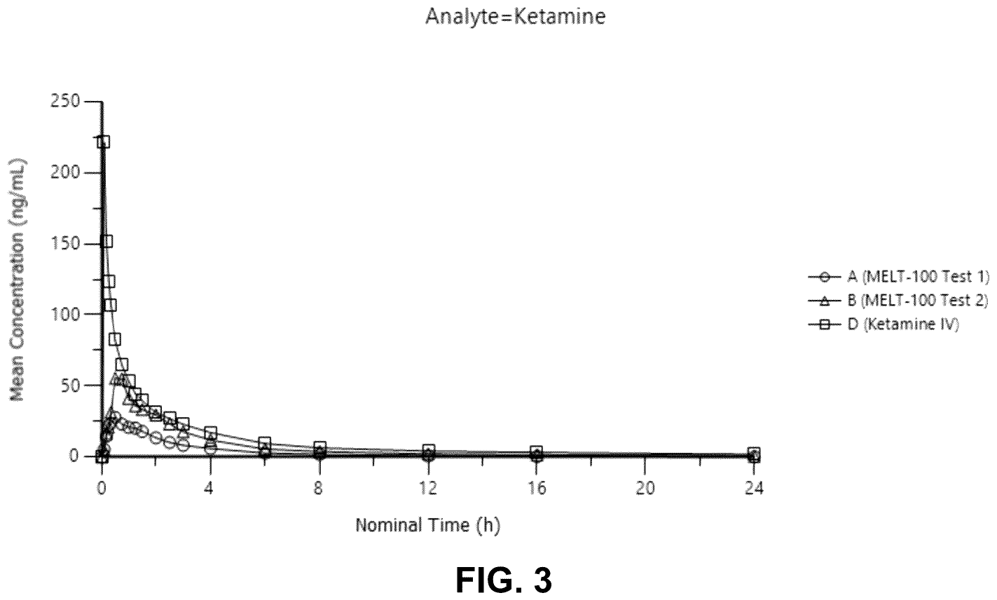

FIG. 4 shows mean plasma concentration-time profiles of the analyte norketamine on the linear scale, from the pharmacokinetics population after administration of MELT-100 midazolam and ketamine formulations, and ketamine IV alone. Open circles depict Treatment A (Test 1)=A single MELT-100 sublingual tablet (3 mg midazolam and 25 mg ketamine). Open triangles depict Treatment B (Test 2)=A single MELT-100 sublingual tablet (2×3 mg midazolam and 25 mg ketamine administered sublingually 15 min apart, total dose=6/50 mg). Open squares depict Treatment D (Reference 2)=A single dose of ketamine 18 mg (10 mg/mL in a 20 mL vial) administered as IV over 5 minutes.

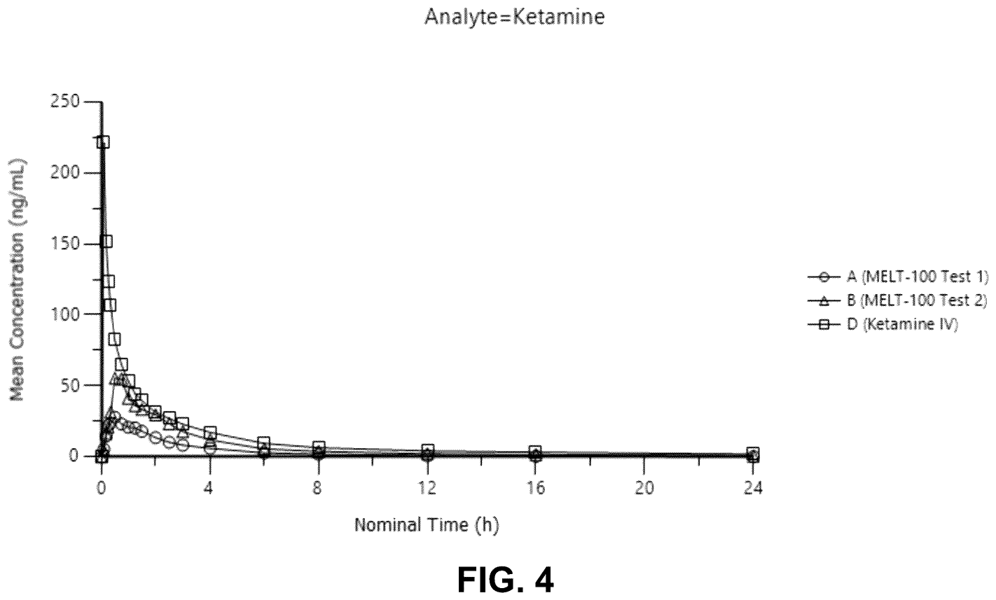

FIG. 5 shows a plot of a Kaplan-Meier Curve of time to achieve preoperative target sedation level for the all-treated analysis set of the Phase 3 MELT-300 study. It represents the time from the administration of study drug until the first Ramsay Sedation Scale (RSS) score of 2 or 3, calculated in minutes. The y-axis shows event-free survival (% of subjects who have not achieved the preoperative target sedation level) and the x-axis shows the number of minutes since administration of the study drug. Calculations were for subjects dosed with MELT-300 who achieve a preoperative RSS score of 2 or 3 using the Kaplan-Meier (KM) method. Analysis excluded subjects who receive preoperative rescue sedation medication.

### DETAILED DESCRIPTION

Discussed below are components of aspects of methods disclosed herein. These and other materials are disclosed herein, and it is understood that when combinations, subsets, interactions, groups, etc., of these materials are disclosed that while specific reference of each various individual and collective combinations and permutation of these components may not be explicitly disclosed, each is specifically contemplated and described herein.

Other aspects and iterations of the invention are described more thoroughly below.

### 1. Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range: i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The terms “anesthetic,” “anesthesia,” “anesthesiology” and the like refer herein to substances, compounds, processes or procedures that induce insensitivity to pain such as a temporary loss of sensation.

The term “conscious sedation,” which for the purposes of this application, may be used inter-changeably with the term “procedural sedation”, and is used herein to refer to an induced state of sedation characterized by a minimally depressed consciousness such that the patient is able to continuously and independently maintain a patent airway, retain protective reflexes, and remain responsive to verbal cues and/or tactile or physical stimulation.

Conscious sedation is typically performed/induced to decrease the level of anxiety in a patient and to elicit an improved degree of cooperation from the patient prior to or during a procedure. Conscious sedation, therefore, refers to a condition that is medically different and distinct from deep sedation which is the next level of sedation defined as depression of consciousness when the patient's ability to independently maintain ventilatory function may be impaired and he or she cannot be easily aroused; however, the patient will still purposefully respond to repeated or painful stimulation.

Conscious sedation is also clearly distinguishable for the purposes of the present application from the lower level of sedation (i.e., minimal sedation when the patient is able to maintain a normal response to verbal stimuli) as well as the highest level of sedation (i.e., general anesthesia when there is no response from the patient even with painful stimulus).

The term “pre-sedation” is defined for the purposes of this application as conscious sedation that is induced some time before a procedure, e.g., between about 5 minutes and about 1 hour prior.

The terms “solvate” and “hydrate” are used herein to indicate that a compound or substance is physically or chemically associated with a solvent for “solvates” such as water (for “hydrates”).

The term “NMDA antagonist” is defined as a compound that inhibits (“antagonizes”) the action of the N-methyl-D-aspartate receptors and is inclusive of both competitive and noncompetitive antagonists, glycine antagonists and uncompetitive channel blockers, as these terms are under-stood by those having ordinary skill in the art.

The term “β-blocker” refers to a compound of any kind that can prevent or reduce the stimulation of the adrenergic receptors responsible for increased cardiac action.

The term “antiemetic” is defined as a drug or medicament that treats, reduces, and/or prevents nausea and/or vomiting.

The term “non-steroid anti-inflammatory drug” or “NSAID” refers to a class of compounds that are free of any steroid moieties yet are capable of providing analgesic, antipyretic and/or anti-inflammatory effects.

The term “antihistamine medicament” refers to any compound that is capable of inhibiting or counteracting the physiological effects of histamine.

The term “polyglycol” is defined as a polymer or oligomer containing several etherglycol link-ages that yields one or more glycols when these linkages are cleaved, e.g., by hydrolysis.

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, and products used for facilitating drug absorption or solubility or for other pharmacokinetic considerations.

The term “binder” refers to a substance or compound that promotes, provides or improves cohesion, i.e., a substance that causes the components of a mixture to cohere to form a solid item that possesses integrity.

The term “troche” refers to a small tablet or lozenge (i.e., a medicated candy intended to be dis-solved in the mouth), typically in a form of a disk, a ball or rhombic in cross-section, comprising medication and processed into a paste and dried.

The term “therapeutically effective amount” is defined as the amount of a compound or pharmaceutical composition that will elicit the biological or medical response of a tissue, system, animal or human that is being sought by the researcher, medical doctor or other clinician.

The term “pharmaceutically acceptable” when used in the context of a carrier, diluent or excipient, refers to a substance that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

The terms “oral administration” and “orally administering” are broadly defined as a route of administration where a medication is taken through the mouth including “sublingual administration” and “buccal administration” where the medication is placed under the tongue or be-tween the gums and the cheek, respectively, to be absorbed by the body, or to be administered sublingually or buccally as a liquid.

### 2. Pharmaceutical Compositions

### 2.1. Midazolam and Ketamine

According to embodiments of the present invention, there are provided pharmaceutical compositions for anesthetic purposes (e.g., comprising midazolam and ketamine). In particular embodiments, the anesthetic purpose is for conscious sedation in a subject (e.g., procedural sedation as described herein). The compositions comprise, consist of, or consist essentially of, a combination of therapeutically effective amounts of a pharmaceutically active compound of a first class and a pharmaceutically active compound of a second class, wherein the pharmaceutically active compound of the first class is midazolam and the pharmaceutically active compound of the second class is ketamine. In certain further embodiments, the compositions optionally comprise, in addition to the above-mentioned pharmaceutically active compounds of the first and second classes (i.e., midazolam and ketamine), at one or more (e.g. at least one) pharmaceutically active compound of a third class.

The pharmaceutically active compound of the first class that is used in a composition as described herein (e.g., a composition of a method as described herein) is midazolam, or a pharmaceutically acceptable salt, hydrate, solvate or N-oxide thereof. Midazolam is of the benzodiazepine drug class, having a benzodiazepine moiety comprising a benzene ring condensed with a diazepine ring and a seven-member heterocycle having two nitrogen atoms. These two nitrogen atoms of a benzodiazepine may be at any positions of the ring (e.g., 1,2-diazepine, 1,3-diazepine or 1,4-diazepine). Midazolam is a 1,4-diazepine. The IUPAC name corresponding to midazolam is: 8-chloro-6-(2-fluorophenyl)-1-methyl-4H-imidazo[1,5-a][1,4]benzodiazepine. Commercially available formulations of midazolam include those having the tradenames: VERSED®, DORMICUM®, and HYPNOVEL®, each of which are contemplated for use herein. In some embodiments (e.g., of a method or composition as described herein), midazolam comprises the chemical structure depicted below as Structure (I):

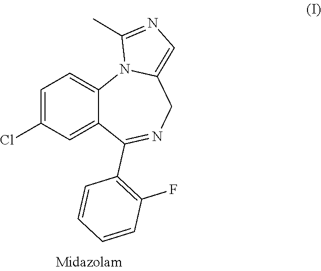 [SMILES: Cc1ncc2n1-c1ccc(Cl)cc1C(c1ccccc1F)=NC2]

The therapeutically effective amount of midazolam in the pharmaceutical composition (e.g., that is administered to the subject) can be between about 0.2 mass % and about 5.0 mass % of the composition. In some embodiments, the therapeutically effective amount of midazolam in the pharmaceutical composition can be between about 1.0 mass % and about 3.0 mass %, for example, about 2.5 mass % of the composition.

The pharmaceutically active compound of the second class that is used in a composition as described herein (e.g., a composition of a method as described herein) is ketamine, or a pharmaceutically acceptable salt, hydrate, solvate or N-oxide thereof (e.g., ketamine hydrochloride). Ketamine is in the NMDA antagonist drug class. The IUPAC name corresponding to ketamine is: 2-(2-chlorophenyl)-2-(methylamino)cyclohexanone. Commercially available formulations of midazolam include those having the tradenames: KETANEST®, KETASET®, or KETALAR® (HCl salt), each of which are contemplated for use herein. In some embodiments, (e.g., of a method or composition as described herein) ketamine comprises the chemical structure depicted below as Structure (II):

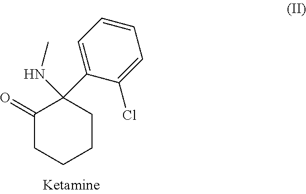 [SMILES: CNC1(c2ccccc2Cl)CCCCC1=O]

The therapeutically effective amount of ketamine in the pharmaceutical composition (e.g., that is administered to the subject) can be between about 1.0 mass % and about 10.0 mass % of the composition. In some embodiments, the therapeutically effective amount of ketamine in the pharmaceutical composition can be between about 4.0 mass % and about 6.0 mass %, for example, about 5.0 mass % of the composition.

Accordingly, in some embodiments of a pharmaceutical composition as described herein, the combined quantities of both the midazolam and the ketamine, taken together, are between about 1.2 mass % and about 15.0 mass % of the composition, such as between about 3.0 mass % and about 12.0 mass %, for example, about 10.0 mass % of the composition.

In particular embodiments of the pharmaceutical composition, the weight ratio of midazolam to ketamine is about 1:5 to about 1:20. In some embodiments, the weight ratio of midazolam to ketamine is about 1:5. In some embodiments, the weight ratio of midazolam to ketamine is about 1:6. In some embodiments, the weight ratio of midazolam to ketamine is about 1:7. In some embodiments, the weight ratio of midazolam to ketamine is about 1:8. In some embodiments, the weight ratio of midazolam to ketamine is about 3:25. In some embodiments, the weight ratio of midazolam to ketamine is about 1:9. In some embodiments, the weight ratio of midazolam to ketamine is about 1:10. In some embodiments, the weight ratio of midazolam to ketamine is about 1:11. In some embodiments, the weight ratio of midazolam to ketamine is about 1:12. In some embodiments, the weight ratio of midazolam to ketamine is about 1:13. In some embodiments, the weight ratio of midazolam to ketamine is about 1:14. In some embodiments, the weight ratio of midazolam to ketamine is about 1:15. In some embodiments, the weight ratio of midazolam to ketamine is about 1:16. In some embodiments, the weight ratio of midazolam to ketamine is about 3:50. In some embodiments, the weight ratio of midazolam to ketamine is about 1:17. In some embodiments, the weight ratio of midazolam to ketamine is about 1:18. In some embodiments, the weight ratio of midazolam to ketamine is about 1:19. In some embodiments, the weight ratio of midazolam to ketamine is about 1:20.

In some embodiments, the pharmaceutical composition may comprise from about 1 mg to about 10 mg of midazolam, such as about 1 mg, 2 mg, 3 mg, 4 mg, 5 mg, 6 mg, 7 mg. 8 mg, 9 mg, or about 10 mg of midazolam. In some embodiments, the pharmaceutical composition may comprise from about 1 mg to about 2 mg, about 1 mg to about 4 mg, about 1 mg to about 6 mg, about 1 mg to about 8 mg, about 1 mg to about 10 mg, about 2 mg to about 10 mg, about 4 mg to about 10 mg, about 6 mg to about 10 mg, about 8 mg to about 10 mg, about 1 mg to about 5 mg, or about 2 mg to about 4 mg of midazolam.

In some embodiments, the pharmaceutical composition may comprise from about 20 mg to about 80 mg of ketamine, such as about 20 mg, 25 mg. 30 mg, 35 mg, 40 mg, 45 mg, 50 mg, 55 mg, 60 mg, 65 mg, 70 mg. 75 mg, or about 80 mg of ketamine. In some embodiments, the pharmaceutical composition may comprise from about 20 mg to about 30 mg, about 20 mg to about 40 mg, about 20 mg to about 50 mg, about 20 mg to about 60 mg, about 20 mg to about 70 mg, about 20 mg to about 80 mg, about 30 mg to about 80 mg, about 40 mg to about 80 mg, about 50 mg to about 80 mg, about 60 mg to about 80 mg, about 70 mg to about 80 mg, about 20 mg to about 50 mg, about 30 mg to about 70 mg, or about 40 mg to about 60 mg of ketamine.

In specific embodiments, the pharmaceutical composition comprises about 3 mg of midazolam and about 25 mg of ketamine. In specific embodiments, the pharmaceutical composition comprises about 3 mg of midazolam and about 50 mg of ketamine.

### 2.2. Optional Pharmaceutically Active Compounds

A pharmaceutical composition as described herein may further comprise one or more (e.g., at least one) pharmaceutically active compound of a third class. The purpose of including a third class is for alleviation or control of a subject's potential sensitivity to the pharmaceutical composition (e.g., sensitivity to midazolam and/or ketamine, or to sublingual administration generally). In some embodiments, the pharmaceutically active compound of the third class is selected from a benzodiazepine receptor antagonist, non-benzodiazepine based sedative, β-blocker, α-2-adrenergic agonist, pain reliever, antiemetic medicament, non-steroid anti-inflammatory drug (NSAID), or antihistamine medicament. In some embodiments, more than one pharmaceutically active compound of the third class is included in the composition (e.g., for different purposes, e.g., an NSAID, a β-blocker, and an antiemetic). In some embodiments, more than one pharmaceutically active compound of the third class may be included in the composition for the same purpose (e.g., multiple β-blockers may be included, e.g., along with multiple antihistamine medicaments).

In certain situations a patient may be sensitive to benzodiazepines, generally, or to midazolam, specifically (e.g., may become excessively drowsy). For such patients, provided herein are certain further embodiments of the midazolam-containing pharmaceutical compositions described herein, further comprise an amount of a benzodiazepine receptor antagonists. Such a receptor antagonist may counteract the effect of midazolam after the surgical procedure is complete, effectively providing a slow release feature. In some embodiments, the one or more pharmaceutically active compound of a third class comprises a benzodiazepine receptor antagonist, or a pharmaceutically acceptable salt, hydrate, solvate or N-oxide thereof. In some embodiments, the benzodiazepine receptor antagonist is selected from an antagonist of the commercially available formulations: ANEXATE®, ROMAZICON®. Other benzodiazepine receptor antagonists known to a skilled artisan are also suitable for the composition. The use of said antagonists is also envisioned as a routine practice (i.e., not just for sensitive patients), for example, in situations when a larger than typical or usual dosage of midazolam is medically indicated, or recommended, or necessary.

In some embodiments, the one or more pharmaceutically active compound of a third class comprises a non-benzodiazepine based sedative, or a pharmaceutically acceptable salt, hydrate, solvate or N-oxide thereof. In some embodiments, the benzodiazepine based sedative is selected from eszopiclone, ramelteon, zolpidem, or zaleplon. Other benzodiazepine based sedatives known to a skilled artisan are also suitable for the composition.

In some embodiments, the one or more pharmaceutically active compound of a third class comprises a β-blocker (e.g., as described herein), or a pharmaceutically acceptable salt, hydrate, solvate or N-oxide thereof. In some embodiments, the β-blocker is selected from metoprolol, propranolol, acebutolol, nadolol, atenolol, betaxolol, esmolol, bisoprolol fumarate, carvedilol, nebivolol, penbutolol, timolol, or sotalol. Each of these is corresponds to a commercially available formulation as shown in TABLE 1, which also discloses IUPAC chemical names. Other aβ-blockers known to a skilled artisan are also suitable for the composition.

TABLE 1
Examples of β-Blockers That Can Be Used in Composition

| Compound | Chemical Name (IUPAC) | Trade Name(s) |
|---|---|---|
| Metoprolol | 1-[4-(2-methoxyethyl)phenoxy]-3-(propan-2- | LOPRESSOR ®; TOPROL ® |
| | ylamino)propan-2-ol | |
| Propranolol | 1-naphthalen-1-yloxy-3-(propan-2-ylamino)propan-2-ol | CIPLA ®; INDERAL ®; and many |
| | | others |
| Acebutolol | N-[3-acetyl-4-[2-hydroxy-3-(propan-2- | SECTRAL ®; PRENT ® |
| | ylamino)propoxy]phenyl]butanamide | |
| Nadolol | (2R,3S)-5-[3-(tert-butylamino)-2-hydroxypropoxy]-1,2,3,4- | CORGARD ® |
| | tetrahydronaphthalene-2,3-diol | |
| Atenolol | 2-[4-[2-hydroxy-3-(propan-2- | TENORMIN ® |
| | ylamino)propoxy]phenyl]acetamide | |
| Betaxolol | 1-[4-[2-(cyclopropylmethoxy)ethyl]phenoxy]-3-(propan-2- | KERLONE ®; BETOPTIC ® and |
| | ylamino)propan-2-ol | others |
| Esmolol | methyl 3-[4-[2-hydroxy-3-(propan-2- | BREVIBLOC ® |
| | ylamino)propoxy]phenyl]propanoate | |
| Bisoprolol | (E)-but-2-enedioic acid; 1-(propan-2-ylamino)-3-[4-(2- | ZEBETA ® |
| fumarate | propan-2-yloxyethoxymethyl)phenoxy]propan-2-ol | |
| Carvedilol | 1-(9H-carbazol-4-yloxy)-3-[2-(2- | COREG ®; CARVIL ®; and |
| | methoxyphenoxy)ethylamino]propan-2-ol | many others |
| Nebivolol | 1-(6-fluoro-3,4-dihydro-2H-chromen-2-yl)-2-[[2-(6-fluoro- | NEBILET ®; BYSTOLIC ® |
| | 3,4-dihydro-2H-chromen-2-yl)-2- | |
| | hydroxyethyl]amino]ethanol | |
| Penbutolol | (2S)-1-(tert-butylamino)-3-(2-cyclopentylphenoxy)propan-2- | LEVATOL ®, |
| | ol | LEVATOLOL ®; and many others |
| Timolol | (2S)-1-(tert-butylamino)-3-[(4-morpholin-4-yl-1,2,5- | TIMOPTIC ®; BETIMOL ®; and |
| | thiadiazol-3-yl)oxy]propan-2-ol | many others |
| Sotalol | N-[4-[1-hydroxy-2-(propan-2- | BETAPACE ® and many others |
| | ylamino)ethyl]phenyl]methanesulfonamide | |

In some embodiments, the one or more pharmaceutically active compound of a third class comprises an α-2-adrenergic agonist, or a pharmaceutically acceptable salt, hydrate, solvate or N-oxide thereof. In some embodiments, the α-2-adrenergic agonist is dexmedetomidine hydrochloride. Other α-2-adrenergic agonists known to a skilled artisan are also suitable for the composition.

In some embodiments, the one or more pharmaceutically active compound of a third class comprises a pain reliever, or a pharmaceutically acceptable salt, hydrate, solvate or N-oxide thereof. In some embodiments, the pain reliever is acetaminophen. Other pain relievers known to a skilled artisan are also suitable for the composition.

In some embodiments, the one or more pharmaceutically active compound of a third class comprises an antiemetic medicament (e.g., as described herein), or a pharmaceutically acceptable salt, hydrate, solvate or N-oxide thereof. In some embodiments, the antiemetic medicament is selected from ondansetron, dolasetron, granisetron, palonosetron, promethazine, imenhydrinate, or meclizine. Each of these corresponds to a commercially available formulation as shown in TABLE 2, which also discloses chemical names of such compounds. Other antiemetics known to a skilled artisan are also suitable for the compositions.

TABLE 2
Examples of Antiemetics That Can Be Used in Compositions

| Compound | Chemical Name (IUPAC) | Trade Name(s) |
|---|---|---|
| Ondansetron | (RS)-9-methyl-3-[(2-methyl-1H-imidazol-1-yl)methyl]- | ZOFRAN ®; ONDISOLV ® |
| | 2,3-dihydro-1H-carbazol-4(9H)-one | |
| Dolasetron | (2a,6a,8a,9a(3)-octahydro-3-oxo-2,6-methano-2H- | ANZEMET ® |
| | quinolizin-8-yl-1H-indole-3-carboxylate | |
| | monomethanesulfonate, monohydrate | |
| Granisetron | 1-methyl-N-((1R,3r,5S)-9-methyl-9- | KYTRIL ® |
| | azabicyclo[3.3.1]nonan-3-yl)-1H-indazole-3-carboxamide | |
| Palonosetron | (3aS)-2-[(3S)-1-azabicyclo[2.2.2]oct-3-yl]-2,3,3a,4,5,6- | ALOXI ® |
| | hexahydro-1H-benz[de]isoquinolin-1-one | |
| Promethazine | (RS)-N,N-dimethyl-1-(1OH-phenothiazin-10- | PHENERGAN ® |
| | yl)propan-2-amine | |
| Dimenhydrinate | 2-benzhydryloxy-N,N-dimethylethanamine; 8-chloro-1,3- | DRAMAMINE ®; GRAVOL ®; |
| | dimethyl-7H-purine-2,6-dione | VOMEX ®; and many others |
| Meclizine | (RS)-1-[(4-chlorophenyl)-phenylmethyl]-4-[(3- | BONINE ®; BONAMINE ®; |
| | methylphenyl)methyl]piperazine | ANTIVERT ®; and many others |

In some embodiments, the one or more pharmaceutically active compound of a third class comprises a non-steroid anti-inflammatory drug (NSAID) (e.g., as described herein or other NSAIDs known to a skilled artisan are also suitable for the composition).

In some embodiments, the one or more pharmaceutically active compound of a third class comprises an antihistamine medicament (e.g., as described herein). In some embodiments, the antihistamine medicament is selected from hydroxyzine pamoate, hydroxyzine hydrochloride, diphenhydramine hydrochloride, meclizine, chlorpheniramine, clemastine, promethazine, or prochlorperazine. Other antihistamine medicaments known to a skilled artisan are also suitable for the composition.

The therapeutically effective amount of the one or more pharmaceutically active compound of the third class in the pharmaceutical composition (e.g., that is administered to the subject) can be between about 0.1 mass % and about 5.0 mass % of the composition. In some embodiments, the therapeutic effective amount of the one or more pharmaceutically active compound of the third class can be between about 1.0 mass % and about 4.0 mass %, for example, about 2.5 mass % of the composition.

Accordingly, in some embodiments of a pharmaceutical composition as described herein, the combined quantities of all the pharmaceutically active compounds (i.e., midazolam, ketamine, and all of the one or more pharmaceutically active compounds of the third class) taken together in the composition can be between about 1.3 mass % and about 20.0 mass % of the composition, such as between about 3.0 mass % and about 12.0 mass %, for example, about 10.0 mass % of the composition. Those having ordinary skill in the art will determine the most appropriate quantities of each the pharmaceutically active compound that are within the above-mentioned ranges and that are most suitable for a particular patient. In some embodiments, the mass ratios between the pharmaceutically active compounds as shown in TABLE 3 may be used for compositions comprising midazolam, ketamine hydrochloride, and the β-blocker propranolol hydrochloride:

TABLE 3
Exemplary Mass Ratios between Midazolam, Ketamine Hydrochloride
and Propranolol Hydrochloride in a Composition

| | | Ketamine | Propranolol |
|---|---|---|---|
| Ratios | Midazolam | Hydrochloride | Hydrochloride |
| Between about | 1 | 2 | 1 |
| and about | 1 | 10 | 1 |
| Such as between about | 1 | 4 | 1 |
| and about | 1 | 6 | 1 |
| For example | 1 | 5 | 1 |

In specific embodiments, the pharmaceutical composition as described herein comprises midazolam, ketamine, and ondansetron. In some embodiments, the mass ratio of midazolam: ketamine: odansetron is about 3:25:2. In some embodiments, the mass ratio of midazolam: ketamine: odansetron is about 3:50:2.

### 2.3. Matrix Former

The pharmaceutical compositions described herein may further comprise one or more matrix forming ingredients (also referred to herein as “matrix formers”). The matrix formers are used to form the matrix, which disintegrates in a subject's mouth to release the active ingredients. In some embodiments, the matrix forming ingredients may include gelatin, mannitol, cellulose derivatives (e.g., methylcellulose, ethylcellulose, hydroxypropylcellulose, hypromellose, carboxymethylcellulose, etc.), agar agar, carob gum, xanthan gum, pectin, chitosan, starches, trehalose, sucrose, acrylic acid polymers, acrylate polymers. In a preferred embodiment, the matrix forming ingredients may include gelatin, mannitol, and combinations thereof.

The matrix former(s) may be present in the pharmaceutical composition in an amount from about 40% to about 95% by weight of the pharmaceutical composition. For example, the matrix former(s) may be present in the pharmaceutical composition in an amount from about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95%. In some embodiments, the matrix former(s) may be present in the pharmaceutical composition in an amount of about 40%, 45%, 50%, 55%, 60%, 65%, 70%, 75%, 80%, 85%, 90%, or about 95% by weight of the pharmaceutical composition. In a non-limiting embodiment, the matrix former(s) are present in an amount from about 80% to about 95% by weight of the pharmaceutical composition, such as about 90%.

### 2.4. Inactive or Neutral Compounds

The pharmaceutical compositions as described herein may further comprise one or more inactive or neutral compound(s). In some embodiments, the inactive or neutral compound is a pharmaceutically acceptable excipient or carrier. Certain dosage formats described herein may require several pharmaceutically acceptable excipients or carriers. In some embodiment, the pharmaceutically acceptable excipient or carrier is selected from a binder, antioxidant, adjuvant, pH adjuster, synergist or preservative. In some embodiments of a pharmaceutical composition, the mass concentration of the one or more inactive or neutral compound(s) can be between about 80 mass % and about 99 mass % of the pharmaceutical composition, such as between about 85 mass % and about 95 mass %, e.g., about 90 mass %. It is noted that in some embodiments, the pharmaceutical composition does not include and/or does not require a reconstitution diluent for use.

Particular embodiments of the invention are directed to pharmaceutical formulations that are formulated as solid articles suitable for sublingual or oral administration (e.g., orally disintegrating tablets, troches, lozenges, capsules, pills, caps or boluses). Binder(s) and/or excipient(s) are useful or may be required for fabricating the solid articles. The compositions can be prepared by first mixing the pharmaceutically active compounds described above with suitable binder(s) and/or excipient(s) followed by molding or compressing the blend. In some embodiments, the solid article is hard (e.g., a hard lozenge or troche). In some embodiments, the solid article is chewable (e.g., a chewable lozenge or troche). In certain embodiments, the pharmaceutical composition comprises one or more binder(s). In some embodiments, the binder is a polyglycol (e.g., as described herein). In some embodiments, the polyglycol is selected from polyethylene glycol (PEG), polyethylene oxide (POE), methoxypolyethylene glycol, polypropylene glycol, polybutylene glycol, or a derivative thereof. In some embodiments, the binder has a molecular weight that is sufficient to provide the necessary hardness and time for dissolution of the composition form (e.g., a troche). In some embodiments, the molecular weight of the binder is between about 1,000 Daltons and about 8,000 Daltons. In some embodiments, the binder is PEG-1450 or PEG-400. In some embodiments, the binder is a polyglycol derivative selected from:
(a) PEG-laureates or dilaureates (e.g., PEG-10-; PEG-12-; PEG-20-; PEG-32-laurates; PEG-20- or PEG-32-dilaurates; PEG-20-glyceryl-; PEG-30-glyceryl- or PEG-40-glyceryl-laurates; or PEG-80-sorbitan laurate);
(b) PEG-oleates, dioleates or trioleates (e.g., PEG-12-; PEG-15-; PEG-20-; PEG-32; PEG-200- or PEG-400-oleates; PEG-20- or PEG-32-dioleates; PEG-20-trioleate, PEG-25-glyceryl trioleate; PEG-20-glyceryl- or PEG-30-glyceryl-oleates; or PEG-40-sorbitan oleate);
(c) PEG-stearates and distearates (e.g., PEG-15-; PEG-40-; PEG-100-stearates; PEG-32-distearate; or PEG-20-glyceryl stearate);
(d) castor, palm kernel, corn or soya oil derivatives of PEG (e.g., PEG-35-; PEG-40- or PEG-60-castor oils; PEG-40-; PEG-50- or PEG-60-hydrogenated castor oils; PEG-40-palm kernel oil; PEG-60-corn oil; or PEG-30-soya sterol);
(e) other PEG derivatives (e.g., PEG-24- or PEG-30-cholesterol; PEG-25-phytosterol; PEG-6- or PEG-8-caprate/caprylate glycerides; tocopheryl PEG-100 succinate; or PEG-15-100 octylphenol products or PEG-10-100 nonylphenol products); or
(f) other products such as: polyglyceryl-10-laurate; POE-9- or POE-23-lauryl ethers; POE-10- or POE-20-oleyl ethers; POE-20-stearyl ether; polysorbates-20 or 80; polyglyceryl-10-oleate; Tween 40; Tween 60; sucrose monostearate; monolaurate; monopalmitate; or various products of Poloxamer series.
   Other binders known to a skilled artisan are also suitable for the composition.

In some embodiments, the pharmaceutical composition comprises one or more sweeteners. The one or more sweeteners may be selected from the group consisting of acesulfame, aspartame, dextrose, fructose, maltitol, saccharin, sorbitol, sucralose, sucrose, pharmaceutically acceptable salts thereof, and combinations thereof. The one or more sweeteners may be included in the pharmaceutical composition in an amount from about 0.5% to about 2% by weight of the pharmaceutical composition.

In some embodiments, the pharmaceutical composition may comprise a flavoring agent. The flavoring agent may include synthetic or natural flavoring agents. The flavoring agent may include vanillin, menthol, methyl anthranilate, manganite, ethyl 2-methylpentanotate, isopentyl acetate, ethyl decadienoate, gamma-octalactone, allyl benzoate, allyl caproate, allyl hexanoate, d-limonene, ethyl butyrate, 3,7-dimethyl-2,6, octadienal, ethyl methylphenylglycidate, or other flavoring agents known in the art and combinations thereof. The flavoring agent may be present in an amount from about 0.5% to about 5% by weight of the pharmaceutical composition.

In certain embodiments, the pharmaceutical composition comprises one or more excipient(s) (e.g., a pharmaceutically acceptable excipient). In some embodiments, the excipient is selected from peppermint oil, or any natural or artificial fruit, vegetable, flower, beverage, or candy flavor, or a combination thereof. Other excipients known to a skilled artisan are also suitable for the composition.

In certain embodiments, the pharmaceutical composition comprises one or more antioxidant(s). In some embodiments, the antioxidant is selected from atocopherol acetate, acetone sodium bisulfite, acetylcysteine, ascorbic acid, ascorbyl palmitate, butylated hydroxyanisole, butylated hydroxytoluene, cysteine, cysteine hydrochloride, tocopherol natural, tocopherol synthetic, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, or tocopherol. Other antioxidants known to a skilled artisan are also suitable for the composition.

In certain embodiments, the pharmaceutical composition comprises one or more adjuvant(s) and/or synergists(s). In some embodiments, the adjuvant or synergists is selected from citric acid, EDTA (ethylenediaminetetraacetate) and salts thereof, hydroxyquinoline sulfate, phosphoric acid, or tartaric acid. Other adjuvants and synergists known to a skilled artisan are also suitable for the composition.

In certain embodiments, the pharmaceutical composition comprises one or more preservative(s). In some embodiments, the preservative is selected from benzalkonium chloride, benzethonium chloride, benzoic acid and salts thereof, benzyl alcohol, boric acid and salts thereof, cetylpyridinium chloride, cetyltrimethyl ammonium bromide, chlorobutanol, chlorocresol, chorhexidine gluconate, chlorhexidine acetate, cresol, ethanol, imidazolidinyl urea, metacresol, methylparaben, nitromersol, o-phenyl phenol, a paraben, phenol, phenylmercuric acetate/nitrate, propylparaben, sodium benzoate, sorbic acid and salts thereof. β-phenylethyl alcohol, or thimerosal. Other preservatives known to a skilled artisan are also suitable for the composition.

In certain embodiments, the pharmaceutical composition comprises one or more pH adjusters. The pH adjuster may comprise acetic acid, citric acid, fumaric acid, hydrochloric acid, lactic acid, malic acid, nitric acid, propionic acid, phosphoric acid, sodium phosphate, sulfuric acid, tartaric acid, ammonium carbonate, diethanolamine, monoethanolamine, potassium hydroxide, sodium bicarbonate, sodium borate, sodium carbonate, sodium hydroxide, sodium phosphate, trolamine, or combinations thereof. In an example, the pH adjuster comprises citric acid.

The amount of the one or more pH adjusters added will vary depending on the desired pH of the pharmaceutical composition. The pH of the pharmaceutical composition may range from about 3 to about 6, such as from about 3 to about 4, about 3 to about 5, about 3 to about 6, about 4 to about 5, about 4 to about 6, or about 5 to about 6. In some embodiments, the pH of the pharmaceutical composition may be about 3, about 3.5, about 4, about 4.5, about 5, about 5.5, or about 6. In a preferred embodiment, the pH of the pharmaceutical composition is about 3.5.

According to certain additional embodiments, the pharmaceutical compositions comprise, consist of or consist essentially of, a therapeutically effective amount of midazolam and ketamine. In some embodiments, the pharmaceutical composition does not comprise another benzodiazepine (e.g., midazolam is only benzodiazepine in the composition). In some embodiments, the pharmaceutical composition does not comprise another NMDA antagonist (e.g., ketamine is the only NMDA antagonist in the composition).

### 2.5. Vehicles

A vehicle can be used as a medium by which a pharmaceutical composition as described herein is administered to a subject. In some embodiments, the vehicle comprises a polymer. In some embodiments, the polymer is selected from an ester of cellulose (e.g., methyl cellulose or hydroxypropyl methyl cellulose), poly(lactic-co-glycolic acid), polylactic acid, polyglycolide, dextrin, polyacetals, poly(N-(2-hydroxypropyl) methacrylamide), polycaprolactone, or poly-3-hydroxybutyrate. In some embodiments, the vehicle comprises one or more excipient(s), which may be selected from gelatin, sodium saccharin, stevioside, peppermint oil, or any natural or artificial fruit, vegetable, flower, beverage, or candy flavor, or a combination thereof. The use of extended release capsules ensconcing the pharmaceutical formulation, or matrix polymer structures is also provided for. A pharmaceutical composition as described herein can be formulated as a troche, a lozenge, a capsule, a pill, a cap, or a bolus.

In additional embodiments, the pharmaceutical compositions of the present invention may be incorporated into vehicles allowing extended release of the compositions over a period of time. To achieve this effect, the compositions may be combined with polymers forming such vehicles. The product will be extended release capsules ensconcing or enveloping the pharmaceutical formulation, or alternatively, a matrix polymer structure holding the pharmaceutical formulation that is embedded into the matrix.

The vehicle carrying the pharmaceutical formulation may be configured to allow the gradual release of the pharmaceutical formulation over a time period for sedation as described herein, or less than a time period for sedation as described herein (e.g., about 5 minutes or less, about 10 minutes or less, about 15 minutes or less, about 20 minutes or less, about 25 minutes or less, about 30 minutes or less, about 25 minutes or less, about 30 minutes or less, about 35 minutes or less, about 40 minutes or less, or about 45 minutes or less). The rate of release may be uniform throughout the entire period of release; alternatively, those having ordinary skill in the art may formulate the release vehicle in such a way as to allow different rates of release at different times, for example, faster release at the beginning of the process of release and slower at later stages, or vice versa, or in any other way that may be necessary.

The vehicle may be manufactured from any pharmaceutically acceptable polymer that is capable of releasing at least 95 mass % of the pharmaceutical formulation that the vehicle incorporates within the above-mentioned time periods (e.g., a time period for sedation as described herein, or less than a time period for sedation as described herein). In some embodiments, the vehicle may be formulated to ensure the release of at least 97 mass % of the pharmaceutical formulation, for example, at least 99.5 mass %.

Those having ordinary skill in the art will select the most appropriate polymer for making the vehicle. As guidance only, some non-limiting examples of such polymers include, but are not limited to, esters of cellulose, e.g., methyl cellulose and hydroxypropyl methyl cellulose. Other acceptable polymers include, but are not limited to, poly(lactic-co-glycolic acid) (PLGA), polylactic acid, polyglycolide, dextrin, polyacetals, poly(N-(2-hydroxypropyl) methacrylamide), polycaprolactone, and poly-3-hydroxybutyrate.

In some embodiments, the vehicle comprises a water-soluble methylcellulose or hydroxypropyl methylcellulose polymer. In some embodiments, the water-soluble methylcellulose or hydroxypropyl methylcellulose polymer is selected from the METHOCEL® family of products, for example, a hydroxypropyl methylcellulose product METHOCEL®E4M, 20% METHOCEL®K4M, or 10% METHOCEL®K100 or, alternatively and particularly useful for hot melt extrusion, another hydroxypropyl methylcellulose product AFFINISOL™MHPMC (all mentioned hydroxypropyl methylcellulose polymers are available from Dow Chemical Co., Midland, Mich.).

### 2.6. Methods for Fabricating

According to further embodiments, methods for fabricating a pharmaceutical composition as described herein are provided. A one-batch formulation method may be used, where the components of the pharmaceutical composition can be combined in single container; the components may be added to the container simultaneously or consecutively. Alternatively, a two- or multiple-batch method(s) may be used if desired, where each component of the pharmaceutical composition can be combined in separate container followed by combining the contents of each container.

In one exemplary, non-limiting procedure, pre-measured quantities of each ingredient in the form of dry powder can be mixed to form a dry blend followed by mixing it with a pre-molten troche base. The composition can then be molded to form a troche.

In another embodiment, the pharmaceutical composition may be fabricated by a freeze-drying process. The methods include: (a) dosing a formulation comprising a non-gelling matrix forming agent into a preformed mold; (b) dosing a formulation comprising a matrix former into the preformed mold; and (c) freeze drying the formulations dosed in steps (a) and (b) to form the pharmaceutical composition.

In step (a), the formulation comprising a non-gelling matrix forming agent includes the midazolam or a pharmaceutically acceptable salt thereof, ketamine or a pharmaceutically acceptable salt thereof, optionally one or more additional water-soluble inactive ingredients, and water. For example, the one or more additional water-soluble inactive ingredients may include pH adjusters, sweeteners, flavors, and the like. In some examples, mannitol may also be added to the formulation of step (a).

The formulation of step (a) is typically in the form of a solution or suspension. Accordingly, a solvent is also present in the formulation. A suitable solvent can be readily chosen by one of ordinary skill in the art once the final composition of the formulation is known, i.e., pharmaceutically active ingredient, excipient, etc. to be present. Preferred solvents include ethanol, isopropanol, other lower alkanols and water, and, more preferably, water. The amount of solvent, preferably water, present in the formulation of step (a) ranges preferably from about 50% to about 98%, more preferably from about 65% to about 98%, and most preferably from about 75% to about 95% based on weight of the formulation of step (a).

The formulation of step (a) can be made by any conventional method. Most typically, the inactive ingredients may be mixed together at any temperature, though preferably between about 20° C. to about 80° C., to form a solution. The solution may then be cooled to a subambient temperature, if heated above an ambient temperature, preferably from about 1° C. to about 30° C., more preferably from about 2° C. to about 20° C., and most preferably from about 5° C. to about 15° C., at which point the active ingredients may be added.

In some aspects, step (a) is repeated one or more times prior to performing step (b). In this way, additional layers may be formed. There is no limit to the number of layers that may be formed using step (a); however, step (b) must follow the performance of one or more step (a), such that the final layer contains a matrix former.

In step (b), a matrix former is dosed into the preformed mold. The formulation of step (b) is typically in the form of a solution or suspension. Accordingly, a solvent is also present in the formulation. A suitable solvent can be readily chosen by one of ordinary skill in the art once the final composition of the formulation is known, i.e., pharmaceutically active ingredient, excipient, etc. to be present. Preferred solvents include ethanol, isopropanol and water, and more preferably, water. The amount of solvent present in the formulation of step (b) ranges preferably from about 50% to about 98%, more preferably from about 65% to about 98%, and most preferably from about 75% to about 95% based on weight of the formulation of step (b).

The formulation of step (b) can be made by any conventional method. Most typically, the matrix former, solvent and optional ingredients may be mixed together at any temperature, though preferably between about 20° C. to about 80° C., to form a solution. The solution may then be cooled to ambient temperature, if heated above an ambient temperature, preferably from about 15° C. to about 30° C., and more preferably from about 20° C. to about 30° C.

In some aspects, step (b) may be repeated one or more times prior to performing step (c). In this way, additional layers may be formed. Step (b) may be repeated one or more times regardless of whether step (a) is also repeated. Preferably, it is not repeated more than four times without also repeating step (a).

In step (c), the formulations dosed in steps (a) and (b) are freeze dried to form the pharmaceutical composition. In a preferred embodiment, step (c) comprises the sub-steps of (cl) freezing the formulations dosed in steps (a) and (b) and then (c2) freeze drying the formulations dosed in steps (a) and (b) to form the pharmaceutical composition of the present invention. Typically, the dosed formulations in the preformed molds are frozen by any means known in the art, for example by passing them through a liquid nitrogen tunnel, preferably for about one to about ten minutes. One of ordinary skill in the art would readily understand the speed with which to pass them through the tunnel. The dosed formulations in the preformed molds are then freeze dried under vacuum.

In another embodiment, the method may include: (a) forming a formulation comprising a non-gelling matrix forming agent into a preformed mold; (b) forming a formulation comprising a matrix former into the preformed mold; (c) combining the formulations formed in steps (a) and (b) into a single-phase formulation; (d) dosing the single-phase formulation into a preformed mold; and (e) freeze drying the formulation dosed in step (d) to form the pharmaceutical composition.

The formulation of steps (a) and (b) can be made by any conventional method described in the embodiment above.

In step (a), the formulation comprising a non-gelling matrix forming agent includes the midazolam or a pharmaceutically acceptable salt thereof, ketamine or a pharmaceutically acceptable salt thereof, optionally one or more additional water-soluble inactive ingredients, and water. For example, the one or more additional water-soluble inactive ingredients may include pH adjusters, sweeteners, flavors, and the like. In some examples, mannitol may also be added to the formulation of step (a).

The formulation of step (a) is typically in the form of a solution or suspension. Accordingly, a solvent is also present in the formulation. A suitable solvent can be readily chosen by one of ordinary skill in the art once the final composition of the formulation is known, i.e., pharmaceutically active ingredient, excipient, etc. to be present. Preferred solvents include ethanol, isopropanol, other lower alkanols and water, and, more preferably, water. The amount of solvent, preferably water, present in the formulation of step (a) ranges preferably from about 50% to about 98%, more preferably from about 65% to about 98%, and most preferably from about 75% to about 95% based on weight of the formulation of step (a).

The formulation of step (a) can be made by any conventional method. Most typically, the inactive ingredients may be mixed together at any temperature, though preferably between about 20° C. to about 80° C., to form a solution. The solution may then be cooled to a subambient temperature, if heated above an ambient temperature, preferably from about 1° C. to about 30° C., more preferably from about 2° C. to about 20° C., and most preferably from about 5° C. to about 15° C., at which point the active ingredients may be added.

In step (b), a formulation comprising a matrix former is formed. The formulation of step (b) is typically in the form of a solution or suspension. Accordingly, a solvent is also present in the formulation. A suitable solvent can be readily chosen by one of ordinary skill in the art once the final composition of the formulation is known, i.e., pharmaceutically active ingredient, excipient, etc. to be present. Preferred solvents include ethanol, isopropanol and water, and more preferably, water. The amount of solvent present in the formulation of step (b) ranges preferably from about 50% to about 98%, more preferably from about 65% to about 98%, and most preferably from about 75% to about 95% based on weight of the formulation of step (b).

The formulation of step (b) can be made by any conventional method. Most typically, the matrix former, solvent and optional ingredients may be mixed together at any temperature, though preferably between about 20° C. to about 80° C., to form a solution. The solution may then be cooled to ambient temperature, if heated above an ambient temperature, preferably from about 15° C. to about 30° C., and more preferably from about 20° C. to about 30° C.

In step (e), the formulations made in steps (a) and (b) and dosed in step (d) are freeze dried to form the pharmaceutical composition. In a preferred embodiment, step (e) comprises the sub-steps of (e1) freezing the formulation dosed in step (d) and then (e2) freeze drying the formulations dosed in step (d) to form the pharmaceutical composition of the present invention. Typically, the dosed formulations in the preformed molds are frozen by any means known in the art, for example by passing them through a liquid nitrogen tunnel, preferably for about one to about ten minutes. One of ordinary skill in the art would readily understand the speed with which to pass them through the tunnel. The dosed formulation in the preformed molds are then freeze dried under vacuum.

### 3. Subjects

The pharmaceutical compositions described herein can be administered to a subject (e.g., a patient) in need of conscious sedation, procedural sedation, and/or pre-sedation, and in general for any kind of non-general anesthesia, by various local administrations. More specifically, the pharmaceutical compositions described herein may be prescribed by ordinarily skilled medical practitioners such as physicians, as the means of conscious sedation or pre-sedation. In certain embodiments, the subject experiences or expects to experience prior to or during a medical or surgical procedure any one of: anxiety (e.g., high anxiety), panic attack (e.g., bouts of panic attacks), disquietude, apprehension, angst, or a feeling of psychological discomfort or distress. In some embodiments, the subject is a human. In some embodiments, the human subject is of any age. In some embodiments, the subject is a child. In some embodiments, the subject is an adolescent. In some embodiments, the subject is an adult. In other contemplated embodiments, the subject is not human.

### 3.1. Subject Procedures

A pharmaceutical composition as described herein is administered to a subject prior to an outpatient surgery or medical procedure, both invasive and non-invasive. In some embodiments, the outpatient surgery or medical procedure is selected from an ophthalmic surgery, dental procedure, urological procedure, obstetric and gynecological procedure, gastrointestinal procedure, otolaryngological procedure, cosmetic surgery procedure, dermatological procedure, podiatric procedure, orthopedic procedure, emergency medical treatment, psychiatric treatment, or veterinarian procedure. In some embodiments, the outpatient or medical procedure comprises a cataract procedure, optionally a cataract extraction procedure. In some embodiments, the outpatient or medical procedure comprises lens replacement. In some embodiments, the outpatient or medical procedure is a cataract extraction with lens replacement (CELR) procedure.

Specific representative examples of surgeries or medical procedure that are amenable to use of a pharmaceutical composition as described herein include, without limitation, cataract surgery, glaucoma surgery, corneal surgery, eyelid surgery, retinal surgery, tooth extraction, oral surgery, root canal surgery, medical imaging procedures (e.g., MRI or CAT scanning, especially for patients suffering from claustrophobia), biopsy, bone marrow harvesting, colonoscopy, endoscopy and laparoscopy.

### 4. Methods for Procedural Sedation

According to embodiments of the present invention, there are provided methods of inducing procedural sedation (e.g., conscious sedation) in a subject (e.g., a subject as described in Section 3) in need thereof, comprising administering to the subject a pharmaceutical composition as described herein (e.g., as described in Section 2). The procedural sedation may be successful (e.g., achieves a target sedation level (Ramsay Sedation Scale [RSS] level of at least 2 for a time period as described herein (e.g., as described in Section 4.5). A successful procedural sedation may be defined as achieving target sedation level (Ramsay Sedation Scale [RSS] level 2 or 3) by the start of a subject procedure (e.g., outpatient or medical procedure (e.g., surgery or operation)), optionally without a need for rescue sedation medication or intraoperative sedation medication, or wherein the subject is able to complete the surgery (e.g., is a procedural sedation responder).

The procedural sedation may be achieved pre-operatively without a need for rescue sedation (e.g., administration of rescue sedation medication). In some embodiments, the subject achieves a target sedation level (e.g., RSS level 2 or 3) by the start of an outpatient or medical procedure (e.g., surgery or operation) without need for rescue sedation. The procedural sedation may be achieved intraoperatively without a need for rescue sedation (e.g., administration of rescue sedation medication). In some embodiments, the subject maintains a target sedation level (e.g., RSS level 2 or 3) throughout an outpatient or medical procedure (e.g., surgery or operation) without need for rescue sedation. In some embodiments, the subject is able to complete the outpatient or medical procedure (e.g., surgery or operation) without a need for rescue sedation. In some embodiments, the subject is able to complete the outpatient or medical procedure (e.g., surgery or operation) without a need for an intervention, optionally wherein the intervention is other than rescue medication.

The procedural sedation achieved in the subject may have an improved (e.g., reduced) likelihood for experiencing an adverse event (AE) (e.g., treatment emergent adverse event, or adverse event of special interest) in comparison to administration of midazolam or ketamine alone to the subject. The adverse event may be selected from one or more of: ketamine emergence delirium (e.g., hallucinations, unpleasant/bad dreams, vivid imagery, confusional state, excitement, behavior abnormal, or agitation); respiratory AE (e.g., respiratory depression, hypoventilation, dyspnea, oxygen desaturation, respiratory arrest, or airway obstruction); cardiovascular AE (e.g., hypotension, blood pressure increased, hypertension (e.g., new onset or worsened), bradycardia/tachycardia, cardiac arrest, or cardiac decompensation); neurocognitive AE (e.g., confusional state, amnesia, memory impairment, attention impaired, disorientation, or delirium); abuse-related AE; oversedation (e.g., sedation excessive), optionally requiring the use of flumazenil; oral/pharyngeal AE (e.g., changes to tongue, oral cavity (e.g., active infection, mucositis, cold sores, canker sores, vesicles, viral lesions, stomatitis, or periodontal disease) and/or oral/pharyngeal function (e.g., hypoesthesia, taste disorders, or dysphagia)).

The procedural sedation achieved in the subject may have an improved vital sign in comparison to administration of midazolam or ketamine alone to the subject. The improved vital sign may be selected from blood pressure (e.g., mean change from baseline in blood pressure); heart rate (e.g., mean change from baseline in heart rate); respiratory rate (e.g., mean change from baseline in respiratory rate); body temperature (e.g., mean change from baseline in body temperature); or pulse oximetry (e.g., mean change from baseline in pulse oximetry).

### 4.1. Single Dosing

In one aspect, there are provided methods of inducing procedural sedation in a subject, the methods comprising sublingually administering to the subject a pharmaceutical composition as described herein (e.g., comprising midazolam and ketamine), wherein the administration achieves a level of sedation in the subject for procedural sedation that lasts for a time period of 60 minutes or less (e.g., 45 minutes or less). In some embodiments, the subject is administered one dose of a pharmaceutical composition as described herein.

### 4.2. Multiple Dosing

In other aspects, there are provided methods of inducing procedural sedation in a subject, the methods comprising sublingually administering a first dose of a pharmaceutical composition as described herein (e.g., comprising midazolam and ketamine), and sublingually administering a further dose (e.g., second, third, or fourth dose, etc.) of the pharmaceutical composition after the sublingual administration of the first dose, wherein the administration achieves a level of sedation in the subject for procedural sedation that lasts for a time period of 60 minutes or less (e.g., 45 minutes or less). In some embodiments, the subject is administered two doses of a pharmaceutical composition as described herein. In some embodiments, the subject is administered three doses of a pharmaceutical composition as described herein. In some embodiments, the subject is administered four doses of a pharmaceutical composition as described herein.

In certain embodiments, the second dose of the pharmaceutical composition occurs within 30 minutes after administering the first dose of the pharmaceutical composition. In some embodiments, the second dose of the pharmaceutical composition occurs within 25 minutes after administering the first dose of the pharmaceutical composition. In some embodiments, the second dose of the pharmaceutical composition occurs within 20 minutes after administering the first dose of the pharmaceutical composition. In some embodiments, the second dose of the pharmaceutical composition occurs within 15 minutes after administering the first dose of the pharmaceutical composition. In some embodiments, the second dose of the pharmaceutical composition occurs within 10 minutes after administering the first dose of the pharmaceutical composition.

In certain embodiments, the third dose of the pharmaceutical composition occurs within 30 minutes after administering the second dose of the pharmaceutical composition. In some embodiments, the third dose of the pharmaceutical composition occurs within 25 minutes after administering the second dose of the pharmaceutical composition. In some embodiments, the third dose of the pharmaceutical composition occurs within 20 minutes after administering the second dose of the pharmaceutical composition. In some embodiments, the third dose of the pharmaceutical composition occurs within 15 minutes after administering the second dose of the pharmaceutical composition. In some embodiments, the third dose of the pharmaceutical composition occurs within 10 minutes after administering the second dose of the pharmaceutical composition.

In certain embodiments, the fourth dose of the pharmaceutical composition occurs within 30 minutes after administering the third dose of the pharmaceutical composition. In some embodiments, the fourth dose of the pharmaceutical composition occurs within 25 minutes after administering the third dose of the pharmaceutical composition. In some embodiments, the fourth dose of the pharmaceutical composition occurs within 20 minutes after administering the third dose of the pharmaceutical composition. In some embodiments, the fourth dose of the pharmaceutical composition occurs within 15 minutes after administering the third dose of the pharmaceutical composition. In some embodiments, the fourth dose of the pharmaceutical composition occurs within 10 minutes after administering the third dose of the pharmaceutical composition.

### 4.3. Reducing Rescue

In one aspect, there are provided methods of reducing the occurrence of rescue in a subject, the methods comprising sublingually administering a pharmaceutical composition as described herein (e.g., comprising midazolam and ketamine), wherein the administration achieves a level of sedation in the subject for procedural sedation. Rescue of a subject may comprise administration of a rescue sedation medication to the subject. The rescue sedation medication may comprise midazolam or ketamine. Rescue may be performed pre-operatively or intra-operatively. Rescue may be performed when the subject's level of sedation is less than 2, or is 1 on the Ramsay sedation scale (RSS)).

### 4.4. Sedation Levels

Sedation in a subject (e.g., in a method as described herein) may be measured in accordance to one or more sedation scales known to a skilled artisan, for instance, the Ramsay sedation scale (RSS), as summarized in TABLE 4, or the Richmond Agitation Sedation Scale (RASS), or Riker Sedation-Agitation Scale (SAS).

TABLE 4
Ramsay sedation scale (RSS)

| Score | Description |
|---|---|
| 1 | Patient is anxious and agitated or restless, or both |
| 2 | Patient is co-operative, oriented, and tranquil |
| 3 | Patient responds to commands only |
| 4 | Patient exhibits brisk response to light glabellar |
| | tap or loud auditory stimulus |
| 5 | Patient exhibits a sluggish response to light |
| | glabellar tap or loud auditory stimulus |
| 6 | Patient exhibits no response |

In certain embodiments, the level of sedation achieved is measured via the Ramsay sedation scale (RSS). In some embodiments, a subject as described herein achieves a level of sedation as measured by RSS score when they exhibit characteristics as described in TABLE 4.

In some embodiments of a method as described herein, the level of sedation to achieve procedural sedation in a subject is an RSS score of greater than 1 (e.g., an RSS score of 1 may require rescue). In some embodiments, the level of sedation to achieve procedural sedation in a subject is an RSS score of between 2 and 5, inclusive; preferably an RSS score of between 2 and 4, inclusive; and more preferably an RSS score of between 2 and 3, inclusive. In specific embodiments, the level of sedation to achieve procedural sedation in a subject is an RSS score of 2, 3, or 4.

In some embodiments, a subject as described herein requires further sedation when they are anxious, agitated, and/or restless (e.g., RSS score of 1). In some embodiments, a subject as described herein may or may not require further sedation when they are co-operative, oriented, and/or tranquil (e.g., RSS score of 2). In some embodiments, a subject as described herein does not require further sedation when they respond to commands only (e.g., RSS score of 3). In some embodiments, a subject as described herein does not require further sedation when they exhibit a brisk response to light glabellar tap or loud auditory stimulus (e.g., RSS score of 4).

In certain embodiments of a method as described herein, the level of sedation requiring rescue in a subject (e.g., to achieve procedural sedation in the subject) is an RSS score of 1. In certain embodiments, the level of sedation requiring rescue in a subject is an RSS score of 2 or less, wherein the RSS score is falling or not improving over time in the subject.

In certain embodiments of a method as described herein, the decision to rescue a subject is determined via measurement of a pharmacokinetic parameter as described herein (e.g., of midazolam, 1-hydroxymidazolam, ketamine, or norketamine). In some embodiments, the level of sedation requiring rescue is determined via pharmacokinetic parameter as described herein (e.g., of midazolam, 1-hydroxymidazolam, ketamine, or norketamine).

In certain embodiments of a method as described herein, the level of sedation achieved is greater than achieved by administering midazolam alone. In certain embodiments of a method as described herein, the level of sedation achieved is greater than achieved by administering ketamine alone.

### 4.5. Time Periods of Sedation

In certain embodiments of a method as described herein (e.g., a single dosing method, a multiple dosing method, or a method for reducing rescue), the procedural sedation in the subject lasts for a time period of 60 minutes or less. In some embodiments, the procedural sedation lasts for a time period of 55 minutes or less. In some embodiments, the procedural sedation lasts for a time period of 50 minutes or less. In some embodiments, the procedural sedation lasts for a time period of 45 minutes or less. In some embodiments, the procedural sedation lasts for a time period of 40 minutes or less. In some embodiments, the procedural sedation lasts for a time period of 35 minutes or less. In some embodiments, the procedural sedation lasts for a time period of 30 minutes or less. In some embodiments, the procedural sedation lasts for a time period of 25 minutes or less. In some embodiments, the procedural sedation lasts for a time period of 20 minutes or less. In some embodiments, the procedural sedation lasts for a time period of 15 minutes or less. In some embodiments, the procedural sedation lasts for a time period of 10 minutes or less. In preferred embodiments, the procedural sedation lasts for a time period of 45 minutes or less, 30 minutes or less, or 15 minutes or less.

In certain embodiments of a method as described herein (e.g., a single dosing method, a multiple dosing method, or a method for reducing rescue), the procedural sedation in the subject is achieved in a time period of 5 minutes or less. In some embodiments, the procedural sedation is achieved in a time period of 10 minutes or less. In some embodiments, the procedural sedation is achieved in a time period of 15 minutes or less. In some embodiments, the procedural sedation is achieved in a time period of 20 minutes or less. In some embodiments, the procedural sedation is achieved in a time period of 25 minutes or less. In some embodiments, the procedural sedation is achieved in a time period of 30 minutes or less. In some embodiments, the procedural sedation is achieved in a time period of 35 minutes or less. In some embodiments, the procedural sedation is achieved in a time period of 40 minutes or less. In some embodiments, the procedural sedation is achieved in a time period of 45 minutes or less. In some embodiments, the procedural sedation is achieved in a time period of 50 minutes or less. In some embodiments, the procedural sedation is achieved in a time period of 55 minutes or less. In some embodiments, the procedural sedation is achieved in a time period of 60 minutes or less.

In some embodiments, the time period of procedural sedation is measured from the moment of administration of the pharmaceutical composition to the subject. In some embodiments, the time period of procedural sedation is measured from the time point that the subject becomes sedated. In some embodiments, the time period of procedural sedation is measured from the time point that the subject first reaches an RSS score (e.g., that is not zero). In some embodiments, the time period of procedural sedation is measured from the time point that the subject first reaches an RSS score of 1. In some embodiments, the time period of procedural sedation is measured from the time point that the subject first reaches an RSS score of 2. In some embodiments, the time period of procedural sedation is measured from the time point that the subject first reaches an RSS score of 3. In some embodiments, the time period of procedural sedation is measured from the time point that the subject first reaches an RSS score of 4.

### 4.6. Pharmacokinetics (PK) of Midazolam and Ketamine

The sublingual administration of a pharmaceutical composition as described herein produces a pharmacokinetic profile in the subject.

### 4.6.1. Midazolam

In certain embodiments of a method for procedural sedation as described herein (e.g., a single dosing method, a multiple dosing method, or a method for reducing rescue), the sublingual administration results in a Cmax of midazolam in the subject that is lower than a Cmax of midazolam resulting from intravenous administration of an equivalent amount of midazolam. In some embodiments, the sublingual administration results in a Cmax of midazolam in the subject that is at least about 10% lower, at least about 20% lower, at least about 30% lower, at least about 40% lower, at least about 50% lower, at least about 60% lower, at least about 70% lower, or at least about 80% lower than a Cmax of midazolam resulting from intravenous administration of an equivalent amount of midazolam. In preferred embodiments of a single dosing method as described herein, the sublingual administration results in a Cmax of midazolam that is at least 50% lower, at least about 60% lower, at least about 70% lower, or at least about 80% lower, than a Cmax of midazolam resulting from intravenous administration of an equivalent amount of midazolam. In preferred embodiments of a multiple dosing method as described herein, the sublingual administration results in a Cmax of midazolam that is at least 30% lower, at least about 40% lower, at least about 50% lower, or at least about 60% lower, than a Cmax of midazolam resulting from intravenous administration of an equivalent amount of midazolam.

In certain embodiments of a method for procedural sedation as described herein (e.g., a single dosing method, a multiple dosing method, or a method for reducing rescue), the sublingual administration results in a peak concentration (Cmax) of midazolam in the subject of less than or equal to about 140 ng/mL. In some embodiments, the sublingual administration results in a Cmax of midazolam in the subject of less than or equal to about 130 ng/mL. In some embodiments, the sublingual administration results in a Cmax of midazolam in the subject of less than or equal to about 120 ng/mL. In some embodiments, the sublingual administration results in a Cmax of midazolam in the subject of less than or equal to about 110 ng/mL. In some embodiments, the sublingual administration results in a Cmax of midazolam in the subject of less than or equal to about 100 ng/mL. In some embodiments, the sublingual administration results in a Cmax of midazolam in the subject of less than or equal to about 90 ng/ml. In some embodiments, the sublingual administration results in a Cmax of midazolam in the subject of less than or equal to about 80 ng/mL. In some embodiments, the sublingual administration results in a Cmax of midazolam in the subject of less than or equal to about 70 ng/ml. In some embodiments, the sublingual administration results in a Cmax of midazolam in the subject of less than or equal to about 60 ng/mL. In some embodiments, the sublingual administration results in a Cmax of midazolam in the subject of less than or equal to about 50 ng/mL. In some embodiments, the sublingual administration results in a Cmax of midazolam in the subject of less than or equal to about 40 ng/ml. In some embodiments, the sublingual administration results in a Cmax of midazolam in the subject of less than or equal to about 30 ng/mL. In some embodiments, the sublingual administration results in a Cmax of midazolam in the subject of less than or equal to about 20 ng/mL. In some embodiments, the sublingual administration results in a Cmax of midazolam in the subject of less than or equal to about 10 ng/ml. In preferred embodiments of a single dosing method as described herein, the sublingual administration results in a Cmax of midazolam of less than or equal to about 25 ng/ml in the subject. In preferred embodiments of a multiple dosing method as described herein, the sublingual administration results in a Cmax of midazolam of less than or equal to about 60 ng/mL in the subject.

In certain embodiments of a method for procedural sedation as described herein (e.g., a single dosing method, a multiple dosing method, or a method for reducing rescue), the sublingual administration results in an area under the curve up to the last quantified time-point (AUC0-t) of midazolam in the subject that is that is not statistically different from or lower than an AUC0-t of midazolam resulting from intravenous administration of an equivalent amount of midazolam. In some embodiments, the sublingual administration results in an AUC0-t of midazolam in the subject that is at least about 10% lower, at least about 20% lower, at least about 30% lower, at least about 40% lower, or at least about 50% lower than an AUC0-t of midazolam resulting from intravenous administration of an equivalent amount of midazolam.

In certain embodiments of a method for procedural sedation as described herein (e.g., a single dosing method, a multiple dosing method, or a method for reducing rescue), the sublingual administration results in an infinity-extrapolated area under the cure (AUCinf) of midazolam in the subject that is not statistically different from or lower than an AUCinf of midazolam in the subject resulting from intravenous administration of an equivalent amount of midazolam. In some embodiments, the sublingual administration results in an AUCinf that is at least about 10% lower, at least about 20% lower, at least about 30% lower, at least about 40% lower, or at least about 50% lower than an AUCinf of midazolam resulting from intravenous administration of an equivalent amount of midazolam.

In certain embodiments of a method for procedural sedation as described herein (e.g., a single dosing method, a multiple dosing method, or a method for reducing rescue), the sublingual administration results in a half-life (t1/2) of midazolam in the subject that is at least about 1 hr, 2 hr, 3 hr, 4 hr, or 5 hr shorter than a half-life of ketamine resulting from intravenous administration of an equivalent amount of ketamine.

In certain embodiments of a method for procedural sedation as described herein (e.g., a single dosing method, a multiple dosing method, or a method for reducing rescue), the sublingual administration results in a Cmax of 1-hydroxymidazolam in the subject that is greater than a Cmax of 1-hydroxymidazolam resulting from intravenous administration of an equal amount of midazolam. In some embodiments, the sublingual administration results in a Cmax of 1-hydroxymidazolam in the subject is at least about 10% greater, at least about 25% greater, at least about 50% greater, at least about 100% greater, at least about 125% greater, at least about 150% greater, at least about 175% greater, or at least about 200% greater than a Cmax of 1-hydroxymidazolam resulting from intravenous administration of an equal amount of midazolam.

In certain embodiments of a method for procedural sedation as described herein (e.g., a single dosing method, a multiple dosing method, or a method for reducing rescue), the sublingual administration results in a Cmax of 1-hydroxymidazolam in the subject that is greater than a Cmax of 1-hydroxymidazolam that is greater than or equal to about 6 ng/mL, greater than or equal to about 7 ng/ml, greater than or equal to about 8 ng/ml, greater than or equal to about 9 ng/ml, greater than or equal to about 10 ng/mL, greater than or equal to about 11 ng/ml, greater than or equal to about 12 ng/mL, greater than or equal to about 13 ng/mL, greater than or equal to about 14 ng/ml, greater than or equal to about 15 ng/ml, or greater than or equal to about 16 ng/ml. In preferred embodiments of a single dosing method as described herein, the sublingual administration results in a Cmax of 1-hydroxymidazolam in the subject of greater than or equal to about 7 ng/mL in the subject (e.g., ˜7.78 ng/ml). In preferred embodiments of a multiple dosing method as described herein, the sublingual administration results in a Cmax of 1-hydroxymidazolam in the subject of greater than or equal to about 16 ng/mL in the subject (e.g., ˜16.2 ng/ml).

### 4.7. Ketamine

In certain embodiments of a method for procedural sedation as described herein (e.g., a single dosing method, a multiple dosing method, or a method for reducing rescue), the sublingual administration results in a Cmax of ketamine in the subject that is lower than a Cmax of ketamine resulting from intravenous administration of an equivalent amount of ketamine. In some embodiments, the sublingual administration results in a Cmax of ketamine in the subject that is at least about 10% lower, at least about 20% lower, at least about 30% lower, at least about 40% lower, at least about 50% lower, at least about 60% lower, at least about 70% lower, or at least about 80% lower than a Cmax of ketamine resulting from intravenous administration of an equivalent amount of ketamine. In preferred embodiments of a single dosing method as described herein, the sublingual administration results in a Cmax of ketamine in the subject that is at least about 50% lower, at least about 60% lower, at least about 70% lower, or at least about 80% lower, than a Cmax of ketamine resulting from intravenous administration of an equivalent amount of ketamine. In preferred embodiments of a multiple dosing method as described herein, the sublingual administration results in a Cmax of ketamine in the subject that is at least about 40% lower, at least about 50% lower, at least about 60% lower, or at least about 70% lower, than a Cmax of ketamine in the subject resulting from intravenous administration of an equivalent amount of ketamine.

In certain embodiments of a method for procedural sedation as described herein (e.g., a single dosing method, a multiple dosing method, or a method for reducing rescue), the sublingual administration results in a peak concentration (Cmax) of ketamine in the subject of less than or equal to about 275 ng/mL. In some embodiments, the sublingual administration results in a Cmax of ketamine in the subject of less than or equal to about 250 ng/mL. In some embodiments, the sublingual administration results in a Cmax of ketamine in the subject of less than or equal to about 225 ng/ml. In some embodiments, the sublingual administration results in a Cmax of ketamine in the subject of less than or equal to about 200 ng/mL. In some embodiments, the sublingual administration results in a Cmax of ketamine in the subject of less than or equal to about 175 ng/mL. In some embodiments, the sublingual administration results in a Cmax of ketamine of less than or equal to about 150 ng/ml in the subject. In some embodiments, the sublingual administration results in a Cmax of ketamine in the subject of less than or equal to about 150 ng/mL. In some embodiments, the sublingual administration results in a Cmax of ketamine in the subject of less than or equal to about 125 ng/mL. In some embodiments, the sublingual administration results in a Cmax of ketamine in the subject of less than or equal to about 100 ng/ml. In some embodiments, the sublingual administration results in a Cmax of ketamine in the subject of less than or equal to about 75 ng/mL. In some embodiments, the sublingual administration results in a Cmax of ketamine in the subject of less than or equal to about 50 ng/mL in the subject. In preferred embodiments of a single dosing method as described herein, the sublingual administration results in a Cmax of ketamine of less than or equal to about 50 ng/mL in the subject (e.g., 31.2 ng/ml). In preferred embodiments of a multiple dosing method as described herein, the sublingual administration results in a Cmax of ketamine of less than or equal to about 75 ng/mL in the subject (e.g., 63.4 ng/ml).

In certain embodiments of a method for procedural sedation as described herein (e.g., a single dosing method, a multiple dosing method, or a method for reducing rescue), the sublingual administration results in an area under the curve up to the last quantified time-point (AUC0-t) of ketamine in the subject that is lower than an AUC0-t of ketamine resulting from intravenous administration of an equivalent amount of ketamine. In some embodiments, the sublingual administration results in an AUC0-t that is at least about 10% lower, at least about 20% lower, at least about 30% lower, at least about 40% lower, at least about 50% lower, at least about 60% lower, or at least about 70% lower, than an AUC0-t of ketamine resulting from intravenous administration of an equivalent amount of ketamine.

In certain embodiments of a method for procedural sedation as described herein (e.g., a single dosing method, a multiple dosing method, or a method for reducing rescue), the sublingual administration results in an infinity-extrapolated area under the cure (AUCinf) of ketamine in the subject that is lower than an AUCinf of ketamine resulting from intravenous administration of an equivalent amount of ketamine. In some embodiment, the sublingual administration results in an AUCinf that is at least about 10% lower, at least about 20% lower, at least about 30% lower, at least about 40% lower, at least about 50% lower, at least about 60% lower, or at least about 70% lower than an AUCinf of ketamine resulting from intravenous administration of an equivalent amount of ketamine.

In certain embodiments of a method for procedural sedation as described herein (e.g., a single dosing method, a multiple dosing method, or a method for reducing rescue), the sublingual administration results in a half-life (t1/2) of ketamine in the subject that is at least about 1 hr, 2 hr, 3 hr, 4 hr, or 5 hr shorter than a half-life of ketamine resulting from intravenous administration of an equivalent amount of ketamine.

In certain embodiments of a method for procedural sedation as described herein (e.g., a single dosing method, a multiple dosing method, or a method for reducing rescue), the sublingual administration results in a Cmax of norketamine in the subject that is greater than a Cmax of norketamine resulting from intravenous administration of an equivalent amount of ketamine. In some embodiments, the sublingual administration results in a Cmax of norketamine in the subject that is at least about 10% greater, at least about 25% greater, at least about 50% greater, at least about 75% greater, at least about 100% greater, at least about 125% greater, at least about 150% greater, at least about 175% greater, at least about 200% greater, at least about 225% greater, at least about 250% greater, at least about 275% greater, at least about 300% greater, at least about 325% greater, at least about 350% greater, at least about 375% greater, or at least about 400% greater than a Cmax of norketamine resulting from intravenous administration of an equivalent amount of ketamine.

In certain embodiments of a method for procedural sedation as described herein (e.g., a single dosing method, a multiple dosing method, or a method for reducing rescue), the sublingual administration results in a Cmax of norketamine in the subject of greater than a Cmax of norketamine resulting from intravenous administration of an equivalent amount of ketamine. In some embodiments, the sublingual administration results in a Cmax of norketamine in the subject of greater than or equal to about 30 ng/mL. In some embodiments, the sublingual administration results in a Cmax of norketamine in the subject of greater than or equal to about 50 ng/mL. In some embodiments, the sublingual administration results in a Cmax of norketamine in the subject of greater than or equal to about 75 ng/mL. In some embodiments, the sublingual administration results in a Cmax of norketamine in the subject of greater than or equal to about 100 ng/mL. In some embodiments, the sublingual administration results in a Cmax of norketamine in the subject of greater than or equal to about 125 ng/mL. In some embodiments, the sublingual administration results in a Cmax of norketamine in the subject of greater than or equal to about 150 ng/mL. In some embodiments, the sublingual administration results in a Cmax of norketamine in the subject of greater than or equal to about 175 ng/mL. In preferred embodiments of a single dosing method as described herein, the sublingual administration results in a Cmax of norketamine in the subject of greater than or equal to about 100 ng/ml (e.g., about 102 ng/mL). In preferred embodiments of a multiple dosing method as described herein, the sublingual administration results in a Cmax of norketamine in the subject of greater than or equal to about 175 ng/ml in the subject (e.g., 193 ng/ml).

### 4.8. Prodrugs

In other aspects, there are provided methods of inducing procedural sedation in a subject, the methods comprising sublingually administering a pharmaceutical composition comprising a prodrug of 1-hydroxymidazolam and prodrug of norketamine, wherein the administration achieves a level of sedation in the subject for procedural sedation that lasts for a time period of 60 minutes or less (e.g., 45 minutes or less).

In other aspects, there are provided methods of inducing procedural sedation in a subject, the methods comprising sublingually administering a first dose of administering a pharmaceutical composition comprising a prodrug of 1-hydroxymidazolam and prodrug of norketamine, and sublingually administering a further dose (e.g., second dose) of the pharmaceutical composition after the sublingual administration of the first dose, wherein the administration achieves a level of sedation in the subject for procedural sedation that lasts for a time period of 60 minutes or less (e.g., 45 minutes or less).

In other aspects, there are provided methods of reducing the occurrence of rescue in a subject, the methods comprising sublingually administering a pharmaceutical composition comprising a prodrug of 1-hydroxymidazolam and prodrug of norketamine, wherein the administration achieves a level of sedation in the subject for procedural sedation. Rescue may be performed when the subject's level of sedation is a 1 on the Ramsay sedation scale (RSS)).

In some embodiments of a method as described herein, the method comprises administering to the subject a pharmaceutical composition comprising a prodrug of 1-hydroxymidazolam, and a prodrug of norketamine. Such pharmaceutical compositions may be useful for achieving procedural sedation, as described herein.

### 4.9. Reducing Rescue

In one aspect, there are provided methods of reducing the occurrence of rescue in a subject, the methods comprising sublingually administering a pharmaceutical composition comprising midazolam and ketamine, wherein the administration achieves a level of sedation in the subject for procedural sedation. Rescue may be performed when the subject's level of sedation is a 1 on the Ramsay sedation scale (RSS)). Rescue may be performed when the subject's level of sedation is less than 2 (e.g., less than 2, less than 3, less than 4, or less than 5) on the Ramsay sedation scale (RSS)).

In certain embodiments of a method of reducing the occurrence rescue in a subject, the rescue is pre-operative. In certain embodiments, the rescue is intra-operative.

In certain embodiments, the occurrence of rescue is reduced as compared to administration of midazolam alone. In certain embodiment, the occurrence of rescue is reduced as compared to administration of ketamine alone.

In some embodiments, no opioids are administered to the subject when rescue is not performed.

### 5. Routes of Administration

In certain embodiments of a method for procedural sedation as described herein (e.g., a single dosing method, a multiple dosing method, or a method for reducing rescue), administration is to the subject local. In some embodiments, the local administration is by the oral route. In some embodiments, the oral route of local administration is sublingually or buccally. In some embodiments, the pharmaceutical composition is delivered to the patient in the form of a solid delivery vehicle such as a troche, a lozenge, a capsule, a pill, a cap, and a bolus, as described herein. In some embodiments, the pharmaceutical composition is formulated as a liquid item adapted for sublingual or buccal administration (in which case it will include all the pharmaceutically active compounds described above, but no pharmaceutically suitable binder); such liquid formulations may be delivered by any method to be selected by one having ordinary skill in the art of delivery of medications, e.g., via a syringe, dropper or pipette. Such local administration may be used instead of intravenous administration or to complement the latter, as appropriate.

It will be understood by those having ordinary skill in the art that the specific dose level and frequency of dosage for any particular patient may be varied and will depend upon many factors including the activity of the specific compound employed, the metabolic stability and length of action of that compound, the age, body weight, general health, gender, and diet of the subject and the severity of the particular surgery or medical procedure.

### 6. Kits

In additional embodiments, pharmaceutical kits comprising a pharmaceutical composition as described herein are provided. A pharmaceutical kit may comprises a sealed container approved for the storage of pharmaceutical compositions (e.g., solid compositions), the container containing one of the pharmaceutical compositions described herein, and instructions for administering the composition. Such kits can facilitate performance of the methods described herein. When supplied as a kit, the composition may be packaged in one container, or a collection of separate containers.

The packaging of a pharmaceutical composition as described herein may comprise a pack or dispenser device, optionally which may contain one or more unit dosage forms containing the composition. The pack may, for example, comprise metal or plastic foil such as a blister pack.

The different components of the composition may also be packaged in one container, or a collection of separate containers. Said components of the composition may be optionally admixed immediately before use. Such packaging of the components separately can also, in certain instances, permit long-term storage without losing activity of the components.

Kits may also include reagents in separate containers such as, for example, sterile water or saline to be added to a lyophilized pharmaceutically active component of the composition that is packaged separately. For example, sealed glass ampules may contain a pharmaceutically active component of the composition and in a separate ampule, sterile water, sterile saline or sterile each of which has been packaged under a neutral non-reacting gas, such as nitrogen. Ampules may comprise or consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like

### 6.1. Labels and Instructions for Use

In certain embodiments, kits may be supplied with a label and/or instructions for use. The label and/or instructions for use are to be affixed to the container or otherwise enclosed with it. Instructions may be printed on paper or other substrate, and/or may be supplied as an electronic-readable medium or video. A detailed label or detailed instructions may not be physically associated with the kit; instead, a user of the kit may be directed to an Internet web site specified by the manufacturer or distributor of the kit to find said label or instructions.

In certain embodiments, the label comprises instructions for the use of a pharmaceutical composition as described herein in a method as described herein. In specific embodiments, the label comprises instructions for use of a pharmaceutical composition as described herein (e.g., a weight ratio of midazolam to ketamine of 3/25 or 3/50, e.g., 3 mg and 25 mg or 3 mg and 50 mg of midazolam and ketamine, respectively, in the induction of procedural sedation in a subject in need thereof (e.g., for cataract surgery).

### ENUMERATED EMBODIMENTS

Embodiment 1: A method of inducing procedural sedation in a subject, the method comprising sublingually administering a pharmaceutical composition comprising midazolam and ketamine, wherein the administration achieves a level of sedation for procedural sedation that lasts for a time period of 45 minutes or less.

Embodiment 2: The method of embodiment 1, wherein the procedural sedation lasts for a time period of 30 minutes or less.

Embodiment 3: The method of embodiment 1 or 2, wherein the procedural sedation lasts for a time period of 15 minutes or less.

Embodiment 4: The method of any one of embodiments 1-3, wherein the level of sedation achieved is measured via the Ramsay sedation scale.

Embodiment 5: The method of any one of embodiments 1-4, wherein the level of sedation achieved is greater than achieved by administering midazolam alone.

Embodiment 6: The method of any one of embodiments 1-5, wherein the level of sedation achieved is greater than achieved by administering ketamine alone.

Embodiment 7: The method of any one of embodiments 1-6, wherein the weight ratio of midazolam to ketamine in the pharmaceutical composition is about 1:5 to about 1:20.

Embodiment 8: The method of any one of embodiments 1-7, wherein the weight ratio of midazolam to ketamine in the pharmaceutical composition is about 3:25.

Embodiment 9: The method of any one of embodiments 1-8, wherein the pharmaceutical composition comprises about 3 mg of midazolam and about 25 mg of ketamine.

Embodiment 10: The method of any one of embodiments 1-7, wherein the weight ratio of midazolam to ketamine in the pharmaceutical composition is about 3:50.

Embodiment 11: The method of any one of embodiments 1-7 or 10, wherein the pharmaceutical composition comprises about 3 mg of midazolam and about 50 mg of ketamine.

Embodiment 12: The method of any one of embodiments 1-11, wherein the sublingual administration results in a Cmax of midazolam in the subject that is at least about 10% lower, at least about 20% lower, at least about 30% lower, at least about 40% lower, at least about 50% lower, at least about 60% lower, at least about 70% lower, or at least about 80% lower than a Cmax of midazolam resulting from intravenous administration of an equivalent amount of midazolam.

Embodiment 13: The method of any one of embodiments 1-12, wherein the sublingual administration results in a Cmax of midazolam in the subject of less than or equal to about 140 ng/mL.

Embodiment 14: The method of any one of embodiments 1-13, wherein the sublingual administration results in an AUC0-t of midazolam in the subject that is not statistically different from or at least about 10% lower, at least about 20% lower, about least about 30% lower, at least about 40% lower, or at least about 50% lower than an AUC0-t of midazolam resulting from intravenous administration of an equivalent amount of midazolam.

Embodiment 15: The method of any one of embodiments 1-14, wherein the sublingual administration results in an AUCinf of midazolam in the subject that is not statistically different from or at least about 10% lower, at least about 20% lower, at least about 30% lower, at least about 40% lower, or at least about 50% lower than an AUCinf of midazolam resulting from intravenous administration of an equivalent amount of midazolam.

Embodiment 16: The method of any one of embodiments 1-15, wherein the sublingual administration results in a Cmax of 1-hydroxymidazolam in the subject that is at least about 25% greater, at least about 50% greater, at least about 100% greater, or at least about 200% greater than a Cmax of 1-hydroxymidazolam resulting from intravenous administration of an equal amount of midazolam.

Embodiment 17: The method of any one of embodiments 1-16, wherein the sublingual administration results in a Cmax of 1-hydroxymidazolam in the subject that is greater than or equal to about 6 ng/mL.

Embodiment 18: The method of any one of embodiments 1-17, wherein the sublingual administration results in a Cmax of ketamine in the subject that is at least about 10% lower, at least about 20% lower, at least about 30% lower, at least about 40% lower, at least about 50% lower, at least about 60% lower, at least about 70% lower, or at least about 80% lower than a Cmax achieved from intravenous administration of an equal amount of ketamine.

Embodiment 19: The method of any one of embodiments 1-18, wherein the sublingual administration results in a Cmax of ketamine in the subject of less than or equal to about 275 ng/mL.

Embodiment 20: The method of any one of embodiments 1-19, wherein the sublingual administration results in an AUC0-t of ketamine in the subject that is at least about 10% lower, at least about 20% lower, at least about 30% lower, at least 40% about lower, at least about 50% lower, at least about 60% lower, or at least about 70% lower than an AUC0-t of ketamine resulting from intravenous administration of an equivalent amount of ketamine.

Embodiment 21: The method of any one of embodiments 1-20, wherein the sublingual administration results in an AUCinf of ketamine in the subject that is at least about 10% lower, at least about 20% lower, at least about 30% lower, at least about 40% lower, at least about 50% lower, at least about 60% lower, or at least about 70% lower than an AUCinf of ketamine resulting from intravenous administration of an equivalent amount of ketamine.

Embodiment 22: The method of any one of embodiments 1-21, wherein the sublingual administration results in a half-life (t1/2) of ketamine in the subject that is at least 1 hr, 2 hr, 3 hr, 4 hr, or 5 hr shorter than a half-life of ketamine resulting from intravenous administration of an equivalent amount of ketamine.

Embodiment 23: The method of any one of embodiments 1-22, wherein the sublingual administration results in a Cmax of norketamine in the subject that is at least about 10% greater, at least about 25% greater, at least about 50% greater, at least about 100% greater, at least about 150% greater, at least about 200% greater, at least about 300% greater, or at least about 400% greater than a Cmax of norketamine achieved from intravenous administration of an equal amount of ketamine.

Embodiment 24: The method of any one of embodiments 1-23, wherein the sublingual administration results in a Cmax of norketamine in the subject of greater than or equal to about 30 ng/ml.

Embodiment 25: A method of inducing procedural sedation in a subject, the method comprising: sublingually administering to the subject a first dose of a pharmaceutical composition comprising midazolam and ketamine; and sublingually administering to the subject a second dose of the pharmaceutical composition after the sublingual administration of the first dose, wherein the procedural sedation lasts 45 minutes or less.

Embodiment 26: The method of embodiment 25, wherein sublingually administering the second dose of the pharmaceutical composition occurs within 30 minutes after administering the first dose of the pharmaceutical composition.

Embodiment 27: The method of embodiment 25 or 26, wherein sublingually administering the second dose of the pharmaceutical composition occurs within 15 minutes after administering the first dose of the pharmaceutical composition.

Embodiment 28: The method of any one of embodiments 25-27, wherein the method results in a Cmax of midazolam in the subject that is at least about 10% lower, at least about 20% lower, at least about 30% lower, at least about 40% lower, at least about 50% lower, or at least about 60% lower than a Cmax of midazolam resulting from intravenous administration of an equivalent amount of midazolam.

Embodiment 29: The method of any one of embodiments 25-28, wherein the method results in a Cmax of midazolam in the subject of less than or equal to 140 ng/ml.

Embodiment 30: The method of any one of embodiments 25-29, wherein the method results in a Cmax of 1-hydroxymidazolam in the subject that is least about 25% greater, at least about 50% greater, at least about 100% greater, or at least about 200% greater than a Cmax achieved from intravenous administration of an equal amount of midazolam.

Embodiment 31: The method of any one of embodiments 25-30, wherein the method results in a Cmax of 1-hydroxymidazolam in the subject that is greater than or equal to about 10 ng/mL.

Embodiment 32: The method of any one of embodiments 25-31, wherein the method results in a Cmax of ketamine in the subject that is at least about 10% lower, at least about 20% lower, at least about 30% lower, at least about 40% lower, at least about 50% lower, at least about 60% lower, or at least 70% lower than a Cmax achieved from intravenous administration of an equal amount of ketamine.

Embodiment 33: The method of any one of embodiments 25-32, wherein the method results in a Cmax of ketamine in the subject of less than or equal to 275 ng/ml in the subject.

Embodiment 34: The method of any one of embodiments 25-33, wherein the method results in a Cmax of norketamine in the subject that is at least about 10% greater, at least about 25% greater, at least about 50% greater, at least about 100% greater, at least about 150% greater, at least about 200% greater, at least about 300% greater, or at least about 400% greater than a Cmax of norketamine in the subject achieved from intravenous administration of an equal amount of ketamine.

Embodiment 35: The method of any one of embodiments 25-34, wherein the method results in a Cmax of norketamine in the subject of greater than or equal to about 30 ng/ml in the subject.

Embodiment 36: A method of reducing the occurrence of rescue, the method comprising sublingually administering a pharmaceutical composition comprising midazolam and ketamine to achieve procedural sedation in a subject, and wherein rescue is performed when the subject's level of sedation is a 1 on the Ramsay sedation scale.

Embodiment 37: The method of embodiment 36, wherein the rescue is pre-operative.

Embodiment 38: The method of embodiment 36 or 37, wherein the rescue is intra-operative.

Embodiment 39: The method of any one of embodiments 36-38, wherein the occurrence of rescue is reduced as compared to administration of midazolam alone.

Embodiment 40: The method of any one of embodiments 36-39, wherein the occurrence of rescue is reduced as compared to administration of ketamine alone.

Embodiment 41: The method of any one of embodiments 36-40, wherein no opioids are administered to the subject when rescue is not performed.

Embodiment 42: A solid pharmaceutical composition formulated for sublingual and or buccal administration comprising about 3 mg of midazolam and about 50 mg of ketamine.

Embodiment 43: The solid pharmaceutical composition of embodiment 42, further comprising a third pharmaceutically active compound selected from a benzodiazepine receptor antagonist, non-benzodiazepine based sedative, β-blocker, α-2-adrenergic agonist, pain reliever, antiemetic medicament, non-steroid anti-inflammatory drug (NSAID), or antihistamine medicament, or a combination thereof or pharmaceutically acceptable salts, hydrates, solvates or N-oxides thereof.

Embodiment 44: The solid pharmaceutical composition of embodiment 42 or 43, wherein the solid dosage form of the composition is selected from a troche, lozenge, capsule, pill, cap, or bolus.

Embodiment 45: The solid pharmaceutical composition of any one of embodiments 42-44, further comprising a binder.

Embodiment 46: The solid pharmaceutical composition of embodiment 45, wherein the binder is selected from a polyethylene glycol, polyethylene oxide, methoxypolyethylene glycol, polypropylene glycol, polybutylene glycol, PEG-laureates, PEG-dilaureates, PEG-oleates, PEG-dioleates, PEG-trioleates, PEG-stearates, PEG-distearates, castor oil derivatives of PEG, palm kernel oil derivatives of PEG, corn oil derivatives of PEG, soya oil derivatives of PEG, cholesterol derivatives of PEG, phytosterol derivatives of PEG, caprate/caprylate glycerides derivatives of PEG, tocopheryl succinate derivatives of PEG, octylphenol derivatives of PEG, nonylphenol derivatives of PEG, polyglyceryl-10-laurate, polyglyceryl-10-oleate, POE-lauryl ethers, POE-oleyl ethers, POE-stearyl ethers, polysorbates, monostearate, monolaurate and monopalmitate derivatives of sucrose, or products of poly(oxypropylene)-co-poly(propylene oxide) family.

Embodiment 47: The solid pharmaceutical composition of any one of embodiments 42-46, further comprising an excipient selected from a gelatin, sodium saccharin, stevioside, peppermint oil, or any natural or artificial fruit, vegetable, flower, beverage, or candy flavor, or a combination thereof.

Embodiment 48: The solid pharmaceutical composition of any one of embodiments 42-47, for use in a method of inducing sedation in a subject.

Embodiment 49: The solid pharmaceutical composition of any one of embodiments 42-48, for use in the manufacture of medicament for inducing sedation in a subject.

Embodiment 50: The solid pharmaceutical composition of any one of embodiments 42-49, for use in a method according to any one of embodiments 1-41.

### EXAMPLES

The following examples are provided to further elucidate technical features of the present invention, but are not intended to limit the scope of the invention. The examples are for the illustrative purposes only. USP pharmaceutical grade products were used in preparing the formulations described in the Examples below.

### Example 1: MELT-100 Sublingual Tablet Phase I Safety Study

### Introduction

A Phase 1 safety study of the MELT-100 formulation was conducted. MELT-100 is a sublingual, needle- and opioid-free formulation for procedural sedation during cataract surgery. MELT-100 combines fixed doses of midazolam (3 mg) and ketamine (25 mg) in one rapidly dissolving tablet (RDT) that is administered sublingually for procedural sedation. Administration of multiple doses of MELT-100 to the subject was also tested (e.g., 2×3 mg midazolam and 25 mg ketamine).

This Phase 1 study was randomized, single-dose, 4-Period, and a crossover relative bioavailability study comparing the MELT-100 formulation with intravenous (IV) midazolam, and IV ketamine under fasted conditions in healthy volunteers.

### Objectives:

The primary objectives of the study were:
(a) to characterize the single-dose pharmacokinetic (PK) parameters of midazolam and ketamine (and major metabolites) after sublingual administration of MELT-100 and compare to IV midazolam and ketamine formulations;
(b) to characterize the proportionality of exposure to midazolam, ketamine, and their metabolites after administration of MELT-100 1×3/25 and 2×3/25 (6/50 total dose);
(c) to establish early exposure/response models for sedation; and
(d) to assess the safety and tolerability of the MELT-100 formulation.

The secondary objective of the study was
(a) To evaluate the effects of single doses of MELT-100 on ECG parameters (e.g., heart rate, QTcF, PR and QRS intervals)

### Study Methodology:

In this study, healthy adult subjects received under fasted conditions a single dose of MELT-100 sublingual tablet, 3/25 mg (Test 1, Treatment A); two doses of a MELT-100 sublingual tablet, 6/50 mg (2×3/25 mg, each dose [1×3/25 mg] administered sublingually 15 minutes apart) (Test 2, Treatment B); midazolam 3.5 mg IV (Reference 1; Treatment C) administered in 3 increments over 2 minutes of 1.5 mg, 1 mg, and 1 mg (2 minutes between each administration), and ketamine 18 mg IV (Reference 2; Treatment D) administered over 5 minutes, each in 1 of 4 treatment periods followed by a 3-day washout.

After a 35-day screening period, up to 28 eligible subjects were randomized into 1 of 4 treatment sequences (7 subjects in each sequence). Subjects who were withdrawn or withdrew from the study were not replaced. The number of subjects planned, enrolled, completed, and analyzed were 28, 25, 23, and 25 subjects, respectively.

TABLE 5
Tested Substances, Doses and Mode of Administration

| Name | Substance | Dose and Mode of Administration |
|---|---|---|
| Test Product 1 | MELT-100 (midazolam 3 mg and | 3/25 mg (1 × 3/25 mg) sublingual tablet |
| | ketamine 25 mg per sublingual tablet) | |
| Test Product 2 | 2 × MELT-100 (midazolam 3 mg and | 6/50 mg (2 × 3/25 mg) sublingual tablet |
| | ketamine 25 mg per sublingual tablet) | |
| Reference 1 | Midazolam (2 mg/2 mL [1 mg/mL] | 3.5 mg IV (1.5 mg as 1.5 mL; 1 mg as 1 |
| | vial) | mL; 1 mg as 1 mL) |
| Reference 2 | Ketamine (200 mg/20 mL [10 mg/mL] | 18 mg IV |
| | vial) | |

### Duration of Treatment:

Four single-dose treatments were administered, a single dose in each of 4 treatment periods, with a 3-day washout period between doses.

### Diagnosis and Criteria for Inclusion:

Subjects were healthy adult males or females≥55 years of age with a body mass index (BMI) of 18.0 to 32.0 kg/m² (inclusive). Female subjects were surgically sterile or postmenopausal.

### Criteria for Evaluation:

### Pharmacokinetics (PK):

PK blood samples were collected predose (0 hour, within 120 minutes prior to dosing), and 5, 10, 15, 20, 30, and 45 minutes, and at 1, 1.25, 1.5, 2, 2.5, 3, 4, 6, 8, 12, 16, and 24 hours after study drug administration.

PK analysis was performed using noncompartmental methods in Phoenix WinNonlin® (Version 8.1, Certara, L.P.) in conjunction with the internet-accessible implementation of the Pharsight® Knowledgebase Server™ (PKSO; Version 4.0.4, Certara, L.P). PKSO provides protected and structured storage, audit trails, and version control for study data, analyses, and related files, supporting 21 CFR Part 11 compliance. The analysis was performed in accordance with the Statistical Analysis Plan (Version 1.0, 5 Jan. 2021).

During the PK analysis, concentrations below the limit of quantitation (BLQ) up to the time of the first quantifiable concentration were treated as zero. Embedded (values between 2 quantifiable concentrations) and terminal BLQ concentrations were treated as “missing”. Calculation of the PK characteristics were based on actual elapsed times [h] (relative to time of dose).

The following PK parameters were determined for midazolam, 1-hydroxymidazolam, ketamine, and norketamine, as appropriate: maximum concentration, determined directly from individual concentration-time data (Cmax); time to reach maximum concentration (Tmax); the observed terminal rate constant; estimated by linear regression through at least three data points in the terminal phase of the log concentration-time profile (Az); the observed terminal half-life (t1/2); area under the concentration-time curve from time-zero to the time of the last quantifiable concentration; calculated using the linear trapezoidal rule (AUC0-t); area under the concentration-time curve from time-zero extrapolated to infinity (AUCinf); the percentage of AUCinf based on extrapolation (AUCextrap %); last quantifiable concentration (Clast); time of the last quantifiable concentration (Tlast); clearance after extravascular administration for ketamine and midazolam only, after MELT-100 (CL/F); volume of distribution in the terminal phase for parent only, after MELT-100 (Vz/F); clearance after IV administration for ketamine and midazolam only (CL); volume of distribution after IV administration for ketamine and midazolam only (Vz); and molecular weight-corrected metabolite-to-parent ratios for AUCinf (1-hydroxymidazolam/midazolam; norketamine/ketamine).

### Pharmacodynamics:

Pharmacodynamic parameters included sedation, defined as time to onset of sedation and sedation level, assessed using the Ramsay sedation scale (RSS), and cardiodynamics assessed by QTcF, heart rate, and PR and QRS intervals, and T-wave morphology and U-wave presence, extracted from ECGs collected via Holter monitor from predose through 24 hours postdose.

### Safety:

The Investigator evaluated safety using physical and oral examinations, O2 saturation (SpO2), vital sign measurements, clinical laboratory evaluations, electrocardiograms (ECGs), reported or observed adverse events (AEs), and assessments of neurocognitive function. Subjects were monitored for any AEs from the first dose through the end of the study.

### Efficacy:

No efficacy evaluations were performed in this Phase I study.

### Statistical Methods:

### Analysis Populations:

PK Population: All evaluable subjects who were dosed and who had sufficient data to calculate at least 3 of the planned PK parameters.

PK Analysis Population: All subjects in the PK Population for whom Cmax, AUC0-t, and AUCinf were estimated for at least 2 treatments.

Safety Population: Defined as all subjects who received at least 1 dose of study drug.

### Pharmacokinetics

All PK analyses were performed on the data from the PK Analysis Population. Comparisons of the log-transformed PK parameters Cmax, AUC0-t, and AUCinf for midazolam 1-hydroxymidazolam, ketamine, and norketamine among treatments was performed using an analysis of variance (ANOVA) model and the 2 one-sided t-tests procedure. The ANOVA model included factors for sequence, subject within sequence, treatment, and period.

The following comparisons were performed:
(a) MELT-100 Treatment A (Test 1) vs Treatment C (Reference 1) for midazolam and 1-hydroxymidazolam
(b) MELT-100 Treatment A (Test 1) vs Treatment D (Reference 2) for ketamine and norketamine
(c) MELT-100 Treatment B (Test 2) vs Treatment C (Reference 1) for midazolam and 1-hydroxymidazolam
(d) MELT-100 Treatment B (Test 2) vs Treatment D (Reference 2) for ketamine and norketamine
(e) MELT-100 Treatment B (Test 2) vs Treatment A (Test 1) for midazolam, l-hydroxymidazolam, ketamine, and norketamine

### Pharmacodynamics

All pharmacodynamic analyses were performed on data from the Safety Population. The RSS scores were summarized by treatment and by timepoint. Time to onset of sedation was summarized by treatment and presented graphically.

The primary cardiodynamic ECG assessment analysis was based on concentration QTc modeling of the relationship between the concentrations of midazolam and ketamine in MELT-100 (and the metabolites of 1-hydroxymidazolam and norketamine) and change from baseline in QTcF (delQTcF) with the intent to exclude the effect of delQTcF>10 msec at clinically relevant plasma concentrations. In addition, the effect of MELT-100 on the change from baseline in heart rate (HR), and PR and QRS intervals was evaluated at each postdose timepoint (a by-timepoint analysis). An analysis of categorical outliers was performed for changes in heart rate; PR, QRS, and QTcF intervals; T-wave morphology; and U-wave presence.

The relationship between plasma concentrations of midazolam and ketamine (and the metabolites of 1-hydroxymidazolam and norketamine) and the metabolites of 1-hydroxymidazolam and norketamine) and delQTcF was quantified using linear-mixed effects modeling approach with a model selection procedure. The full model had delQTcF as the dependent variable, plasma concentrations of midazolam, ketamine, 1-hydroxymidazolam, and norketamine as the explanatory variates, centered baseline QTcF (i.e., baseline QTcF for individual subject minus the population mean baseline QTcF for all subjects in the same treatment period) as an additional covariate, a fixed intercept, and random effects on intercept and slopes per subject.

### Statistical Methods:

### Safety:

All safety analyses were performed on data from the Safety Population. All quantitative safety data were tabulated with descriptive summary statistics, including the following: arithmetic mean, SD, median, minimum value, maximum values, and number of non-missing observations (n).

Frequency counts and percentage were provided for categorical data. Data were summarized by treatment sequence, treatment, and/or overall, as applicable. All observed baseline/predose and postdose values were summarized by treatment when data were available. Additionally, the corresponding change from baseline/predose assessment to each postdose assessment were also summarized by treatment. All tabulations involving change from baseline/predose comparisons included only those subjects with data at both the baseline/predose and postdose assessments.

Adverse events were coded using the Medical Dictionary for Regulatory Activities (MedDRA, version 22.0, or later) terms and summarized by system organ class (SOC) and preferred term (PT) within SOC. The number and percentage of subjects reporting TEAEs were summarized by reported SOC and PT for each treatment and the population overall. TEAEs were also summarized by maximum severity and nearest relationship to study drug, by SOC and PT for each treatment.

A by-subject AE (including treatment-emergent) data listing, including but not limited to verbatim term, SOC, PT, severity, and relationship to study treatment, was provided. Deaths, SAEs, and other significant AEs, including those leading to early termination of study treatments were listed.

Vital signs, clinical laboratory values (hematology, serum chemistry, and urinalysis), and 12-lead ECG parameters were summarized by treatment including any abnormal values flagged.

Physical examination and oral cavity examination data at each evaluation were listed. Clinically significant abnormal findings were noted in the data listings. Weight and height were summarized. Neurocognitive assessments for each evaluation were listed.

### Summary of Results:

### Pharmacokinetic Results:

Of the twenty-five (25) subjects that were enrolled, 25 subjects were included in the PK Population (for summary tables and figures) and 24 subjects were included in the PK Analysis Population (for statistical analysis).

Twenty-three (23) subjects were included in the statistical comparisons for Treatment A vs. Treatment C, Treatment A vs. Treatment D, and Treatment B vs. Treatment A comparisons. Twenty-four (24) subjects were included in Treatment B vs. Treatment C, and Treatment B vs. Treatment D comparisons.

Subject 219 was discontinued by the physician during Period 2 (Treatment A). Concentration-time data for Treatment A in Period 2 for Subject 219 were retained in the concentration listing but excluded from summary statistics, figures, PK analysis, and subsequent statistical analysis (i.e., Treatment A vs. Treatment C, Treatment A vs. Treatment D, and Treatment B vs. Treatment A). Concentration-time data for Subject 219 in Period 1 (Treatment D) were included in summary statistics, figures, and PK analysis.

Subject 205 was withdrawn from the study during Period 4 (Treatment A) due to an adverse event. Concentration-time data for Treatment A in Period 4 for Subject 205 were retained in the concentration listing but excluded from summary statistics, figures, PK analysis, and subsequent statistical analysis (i.e., Treatment A vs. Treatment C, Treatment A vs. Treatment D, and Treatment B vs. Treatment A). Data from Periods 1 (Treatment C), 2 (Treatment D), and 3 (Treatment B) for Subject 205 were included in the summary statistics, figures, and PK analysis. Subject 205 was excluded from all statistical comparisons for Treatment A but included in comparisons for Treatment B (Treatment B vs. Treatment C, Treatment B vs. Treatment D).

Norketamine quantifiable predose concentrations were observed for Subjects 209 (Treatment A), 218 (Treatment D), and 220 (Treatment D). Quantifiable predose norketamine concentrations for Subjects 209 and 220 were less than 5% of the respective Cmax values and were, therefore, included in the PK analysis without adjustment. The quantifiable predose norketamine concentration for Subject 218 was greater than 5% of the respective Cmax; Subject 218 was subsequently excluded from PK analysis of norketamine for Treatment D.

Treatment a (MELT-100 3/25 mg, Test 1) Vs. Treatment C (Midazolam IV 3.5 mg, Reference 1)

The resulting PK parameters of midazolam after administration of Treatment A, Test 1, and Treatment C, Reference 1 are provided in TABLE 6. Midazolam Cmax, AUC0-t, and AUCinf were approximately 81%, 54%, and 54% lower, respectively, after MELT-100 sublingual tablet 3/25 mg (Treatment A, Test 1) compared to midazolam IV 3.5 mg (Treatment C, Reference 1). Median Tmax was 19 min later for MELT-100 sublingual tablet 3/25 mg (30 min) compared to midazolam IV 3.5 mg (11 min). Mean T1/2 was similar for both treatments (6.75 h for MELT-100 3/25 mg and 7.00 h for midazolam IV 3.5 mg).

The resulting PK parameters of 1-hydroxymidazolam after administration of Treatment A, Test 1, and Treatment C, Reference 1 are provided in TABLE 7. For 1-hydroxymidazolam, Cmax was approximately 37% higher and AUCs were approximately 12% to 15% lower after MELT-100 sublingual tablet 3/25 mg (Treatment A, Test 1) compared to midazolam IV 3.5 mg (Treatment C, Reference 1). Median Tmax was 30 min later for MELT-100 sublingual tablet 3/25 mg (60 min) compared to midazolam IV 3.5 mg (30 min). Mean T1/2 was approximately 3.4 h shorter after for MELT-100 3/25 mg (5.87 h) compared to that after midazolam IV 3.5 mg (9.31 h). Mean metabolite-to-parent ratios (MPR) for AUCinf were higher for MELT-100 3/25 mg (0.294) compared to midazolam IV 3.5 mg (0.142).

A statistical analysis of the natural log-transformed systemic exposure of midazolam and 1-hydroxymidazolam when comparing Treatment A, Test 1, and Treatment C, Reference 1 is provided in TABLE 8.

Treatment B (MELT-100 6/50 mg, Test 2) Vs. Treatment C (Midazolam IV 3.5 mg, Reference 1)

The resulting PK parameters of midazolam after administration of Treatment B, Test 2, and Treatment C, Reference 1 are provided in TABLE 6. Midazolam Cmax was approximately 58% lower after MELT-100 sublingual tablet 6/50 mg (Treatment B, Test 2) compared to midazolam IV 3.5 mg (Treatment C, Reference 1); AUCs were similar for both treatments (geometric mean ratios were 97.61% and 97.65% for AUC0-t and AUCinf, respectively). Median Tmax was 34 min later for MELT-100 sublingual tablet 6/50 mg (45 min) compared to midazolam IV 3.5 mg (11 min). Mean T1/2 was similar for both treatments (6.88 h for MELT-100 6/50 mg and 7.00 h for midazolam IV 3.5 mg) . . .

The resulting PK parameters of 1-hydroxymidazolam after administration of Treatment B, Test 2, and Treatment C, Reference 1 are provided in TABLE 7. For 1-hydroxymidazolam, Cmax, AUC0-t, and AUCinf were approximately 212%, 106%, and 90% higher, respectively, after MELT-100 sublingual tablet 6/50 mg (Treatment B, Test 2) compared to midazolam IV 3.5 mg (Treatment C, Reference 1). Median Tmax was 24 min later for MELT-100 sublingual tablet 6/50 mg (54 min) compared to midazolam IV 3.5 mg (30 min). Mean T1/2 was approximately 2.5 h shorter after for MELT-100 6/50 mg (6.84 h) compared to that after midazolam IV 3.5 mg (9.31 h). Mean metabolite-to-parent ratios (MPR) for AUCinf were higher for MELT-100 6/50 mg (0.280) compared to midazolam IV 3.5 mg (0.142).

A statistical analysis of the natural log-transformed systemic exposure of midazolam and 1-hydroxymidazolam when comparing Treatment B, Test 2, and Treatment C, Reference 1 is provided in TABLE 9.

Treatment a (MELT-100 3/25 mg, Test 1) Vs. Treatment D (Ketamine IV 18 mg, Reference 2)

The resulting PK parameters of ketamine after administration of Treatment A, Test 1 and Treatment D, Reference 2 are provided in TABLE 10. Ketamine Cmax, AUC0-t, and AUCinf were approximately 86%, 75%, and 75% lower, respectively, after MELT-100 sublingual tablet 3/25 mg (Treatment A, Test 1) compared to ketamine IV 18 mg (Treatment D, Reference 2). Median Tmax was 20 min later for MELT-100 sublingual tablet 3/25 mg (30 min) compared to ketamine IV 18 mg (10 min). Mean T1/2 was approximately 5 h shorter after for MELT-100 3/25 mg (5.22 h) compared to that after ketamine IV 18 mg (10.3 h).

The resulting PK parameters of norketamine after administration of Treatment A, Test 1 and Treatment D, Reference 2 are provided in TABLE 11. For norketamine, Cmax, AUC0-t, and AUCinf were approximately 163%, 43%, and 26% higher, respectively, after MELT-100 sublingual tablet 3/25 mg (Treatment A, Test 1) compared to ketamine IV 18 mg (Treatment D, Reference 2). Median Tmax was 28 min later for MELT-100 sublingual tablet 3/25 mg (75 min) compared to ketamine IV 18 mg (47 min). Mean T1/2 was approximately 3.3 h shorter after for MELT-100 3/25 mg (10.5 h) compared to that after ketamine IV 18 mg (13.8 h). Mean metabolite-to-parent ratios (MPR) for AUCinf were higher for MELT-100 3/25 mg (7.06) compared to ketamine IV 18 mg (1.50).

A statistical analysis of the natural log-transformed systemic exposure of ketamine and norketamine when comparing Treatment A, Test 1 and Treatment D, Reference 2 is provided in TABLE 12.

Treatment B (MELT-100 6/50 mg, Test 2) Vs. Treatment D (Ketamine IV 18 mg, Reference 2)

The resulting PK parameters of ketamine after administration of Treatment B, Test 2 and Treatment D, Reference 2 are provided in TABLE 10. Ketamine Cmax, AUC0-t, and AUCinf were approximately 72%, 45%, and 45% lower, respectively, after MELT-100 sublingual tablet 6/50 mg (Treatment B, Test 2) compared to ketamine IV 18 mg (Treatment D, Reference 2). Median Tmax was 21 min later for MELT-100 sublingual tablet 6/50 mg (31 min) compared to ketamine IV 18 mg (10 min). Mean T1/2 was similar for both treatments (8.59 h for MELT-100 6/50 mg and 10.3 h for ketamine IV 18 mg).

The resulting PK parameters of norketamine after administration of Treatment B, Test 2 and Treatment D, Reference 2 are provided in TABLE 11. For norketamine, Cmax, AUC0-t, and AUCinf were approximately 406%, 185%, and 149% higher, respectively, after MELT-100 sublingual tablet 6/50 mg (Treatment B, Test 2) compared to ketamine IV 18 mg (Treatment D, Reference 2). Median Tmax was 21 min later for MELT-100 sublingual tablet 6/50 mg (68 min) compared to ketamine IV 18 mg (47 min). Mean T1/2 was approximately 3.6 h shorter after for MELT-100 6/50 mg (10.2 h) compared to that after ketamine IV 18 mg (13.8 h). Mean metabolite-to-parent ratios (MPR) for AUCinf were higher for MELT-100 6/50 mg (6.74) compared to ketamine IV 18 mg (1.50).

A statistical analysis of the natural log-transformed systemic exposure of ketamine and norketamine when comparing Treatment B, Test 2 and Treatment D, Reference 2 is provided in TABLE 13.

Treatment B (MELT-100 6/50 mg, Test 2) Vs. Treatment a (MELT-100 3/25 mg, Test 1): Proportionality Assessment

The resulting PK parameters of midazolam after administration of Treatment B, Test 2, and Treatment A, Test 1 are provided in TABLE 6. The resulting PK parameters of 1-hydroxymidazolam after administration of Treatment B, Test 2, and Treatment A, Test 1 are provided in TABLE 7. Midazolam Cmax, AUC0-t, and AUCinf were approximately 2.2-fold, 2.1-fold, and 2.1-fold higher, respectively, after MELT-100 sublingual tablet 6/50 mg (Treatment B, Test 2) compared to MELT-100 sublingual tablet 3/25 mg (Treatment A, Test 1). For 1-hydroxymidazolam, Cmax, AUC0-t, and AUCinf were approximately 2.2-fold, 2.3-fold, and 2.3-fold higher, respectively, after MELT-100 sublingual tablet 6/50 mg (Treatment B, Test 2) compared to MELT-100 sublingual tablet 3/25 mg (Treatment A, Test 1). Results suggest that midazolam and 1-hydroxymidazolam exposure increased in a dose proportional manner with an increase in midazolam dose from 3 mg to 6 mg. A statistical analysis of the natural log-transformed systemic exposure of midazolam and 1 hydroxymidazolam when comparing Treatment B, Test 2, and Treatment A, Test 1 is provided in TABLE 14.

The resulting PK parameters of ketamine after administration of Treatment B, Test 2, and Treatment A, Test 1 are provided in TABLE 10. The resulting PK parameters of norketamine after administration of Treatment B, Test 2, and Treatment A, Test 1 are provided in TABLE 11. Ketamine Cmax, AUC0-t, and AUCinf were approximately 2.0-fold, 2.1-fold, and 2.1-fold higher, respectively, after MELT-100 sublingual tablet 6/50 mg (Treatment B, Test 2) compared to MELT-100 sublingual tablet 3/25 mg (Treatment A, Test 1). For norketamine, Cmax, AUC0-t, and AUCinf were approximately 1.9-fold, 2.0-fold, and 2.0-fold higher, respectively, after MELT-100 sublingual tablet 6/50 mg (Treatment B, Test 2) compared to MELT-100 sublingual tablet 3/25 mg (Treatment A, Test 1). Results suggest that ketamine and norketamine exposure increased in a dose proportional manner with an increase in ketamine dose from 25 mg to 50 mg. A statistical analysis of the natural log-transformed systemic exposure of ketamine and norketamine when comparing Treatment B, Test 2, and Treatment A, Test 1 is provided in TABLE 15.

### Safety Results:

### Adverse Events:

A total of 15 (60.0%) subjects experienced 34 AEs; 14 (56.0%) of these subjects reported 21 AEs that were considered treatment-related. Most of the AEs were mild to moderate in severity.

The number of subjects with treatment-related AEs was similar across most of the treatments, with the exception of fewer subjects (2, [8.3%]) with fewer treatment-related AEs (2 events) reported for the midazolam 3.5 mg IV treatment. The number of subjects with treatment-related AEs with the MELT-100 3/25 mg and MELT-1006/50 mg treatments was 4 (16.0%) with 6 events and 5 (20.8%) with 7 events, respectively.

The most frequently reported AEs (>1 subject overall) were headache in 4 (16.0%), hypertension in 3 (12.0%), dizziness in 2 (8.0%), nausea in 2 (8.0%), oxygen saturation decreased in 2 (8.0%), and vessel puncture site pain in 2 (8.0%) subjects.

The most frequently reported treatment-related AEs (>1 subject overall) were headache in 3 (12.0%), dizziness in 2 (8.0%), hypertension in 3 (12.0%), and oxygen saturation decreased in 2 (8.0%) subjects.

The 3 treatment-related AEs of hypertension were reported with ketamine 18 mg IV, and the 2 treatment-related AEs of oxygen saturation decreased were reported with MELT-100 6/50 mg. The treatment-related AEs of headache and dizziness, 4 AEs and 2 AEs respectively, were reported with the treatments that contained midazolam.

Most of the AEs overall were of mild severity for 15 (60.0%) subjects; 3 (12.0%) subjects reported moderate AEs, and 1 (4.0%) subject reported a severe AE.

One subject experienced a severe AE of syncope and a moderate AE of orthostatic hypotension that were considered related to MELT-100 3/25 mg, resolved with IV fluids, and led to the subject being discontinued early from the study.

Adverse events of moderate severity were orthostatic hypotension in 1 (4.0%) subject, contact dermatitis in 1 (4.0%) subject, and dry skin in 1 (4.0%) subject.

All of the AEs reported in this study resolved.

No deaths or other SAEs were reported in subjects in this study.

### Clinical Laboratory Values

No clinically meaningful changes in mean values or trends in shifts from Day-1 to EOS/ET were observed for the hematology and serum chemistry parameters.

None of the out-of-range values for any laboratory test were considered clinically significant.

Vital Signs Mean changes from baseline in systolic and diastolic blood pressures were minimal and similar for the MELT-100 3/25 mg, MELT-100 6/25 mg, and midazolam 3.5 mg IV treatments at each timepoint, were highest at the 10-minute timepoint with the ketamine 18 mg IV treatment, but were minimal for this treatment by the 1-hour postdose timepoint.

Mean changes from baseline in pulse rate were minimal and similar for the MELT-100 3/25 mg and MELT-100 6/25 mg treatments at each timepoint, were highest at the 10-minute postdose timepoint, but were minimal for these treatments at the 1 hour postdose timepoint.

Mean changes from baseline in respiratory rates and pulse oximetry were minimal and similar for most of the treatments at each timepoint, with the exception of a small mean decrease from baseline in pulse oximetry for the midazolam 3.5 mg IV treatment at the 10-minute postdose timepoint and for the MELT-100 6/50 mg treatment at the 30-minute postdose timepoint. The mean changes from baseline in pulse oximetry for each of these treatments were minimal at the 2-hour postdose timepoint.

### Electrocardiograms

The mean (SD), median, and ranges of each of the ECG parameters measured at Screening, Day-1, predose, and EOS/ET were similar and unremarkable at each timepoint.

Most shifts in ECG parameters were unremarkable. One subject had a normal PR duration at Screening and Day-1 that was abnormal 24 hours after receiving midazolam 3.5 mg IV in Period 4, but was not considered clinically significant.

Few subjects had ECG values that were considered outliers. Most of these subjects had QT, QTc, QTcB, and QTcF intervals>450 ms that occurred at baseline, and no changes from baseline for these subjects were >30 ms.

One subject had a QTcB interval of 457 ms 24 hours after receiving MELT-100 3/25 mg in Period 4, which was out-of-range by only 7 ms.

### Physical and Oral Cavity Examinations

Most subjects had normal physical examinations and oral cavity examinations with none of the protocol-defined abnormalities observed. One subject had a healing area of denuded skin surrounded by erythema of the left forearm at ET from an AE of second degree burns of the left forearm in Period 1 that was of mild severity, considered not related to study drug, was treated with topical bacitracin 400 units, neomycin 3.5 mg, polymyxin B 5,000 units ointment, and resolved on 8 Mar. 2021.

### Neurocognitive Assessments

Most subjects had SPMSQ assessments that were unchanged at each timepoint measured. One subject had mild cognitive impairment 1 hour after receiving MELT 100 6/50 mg in Period 3. This subject's SMPSQ was assessed as normal mental functioning 2 hours postdose, and no AEs were reported for this subject in Period 3.

### Ramsay Sedation Scale

At 5 minutes after study medication administration, the vast majority of healthy subjects were rated as having an RSS score of 2-corresponding to “subject is cooperative, oriented, and tranquil”: MELT-100 3/25 mg in 25 subjects (100%), MELT-100 6/50 mg in 24 subjects (100%), midazolam 3.5 mg IV in 22 subjects (91.7%), and ketamine 18 mg IV in 23 subjects (92.0%). At that same 5-minute postdose timepoint, 2 (8.3%) subjects had RSS scores of 3-corresponding to “subject responds to commands only” with midazolam 3.5 mg IV; and 1 (4.0%) subject had an RSS score of 1-corresponding to “subject is anxious and agitated or restless, or both” and 1 (4.0%) subject had an RSS score of 5-corresponding to “subject exhibits a sluggish response to light glabellar tap or loud auditory stimulus” both scores with ketamine 18 mg IV.

No subjects had an RSS scores<2 after the 10-minute postdose timepoint, and no subjects had an RSS score of 6-corresponding to “subject exhibits no response” at any postdose timepoint. Higher levels of sedation (RSS scores>2) were more common at later timepoints, with the highest proportion of subjects with RSS scores>2 postdose observed with midazolam 3.5 mg IV (˜54%), followed by ˜46% of subjects with MELT-100 6/50 mg, ˜28% of subjects with MELT-100 3/25 mg, and ˜8% of subjects with ketamine 18 mg IV.

Although RSS scores of 2 at the 5-minute timepoint for MELT-100 at 3/25 mg and 6/50 mg sublingual doses were inconsistent with the median Tmax for each treatment, higher levels of sedation at the 30- and 35-minute timepoints (RSS scores of 2 through 4) were more consistent with the level of sedation expected at Tmax for MELT-100 sublingual tablets.

### Conclusions:

The purpose of this study was to characterize the single-dose PK parameters and dose proportionality of midazolam and ketamine (and their major metabolites) after sublingual administration of MELT-100 at 3/25 mg and 6/50 mg sublingual doses compared with midazolam 3.5 mg and ketamine 18 mg given IV. The safety and tolerability of MELT-100 were also assessed, as were the effects of MELT-100 on ECG parameters.

A total of 25 subjects were randomized and all received at least 1 dose of study treatment. Of those subjects randomized, 23 subjects completed the study with 2 subjects discontinuing early from the study, 1 due to physician decision (conduct toward the study staff) and 1 due to a treatment-related AE that was treated and resolved prior to discontinuation.

Most AEs were mild to moderate in severity, and the number of subjects with treatment-related AEs for the MELT-100 treatments was low (no more than 5 subjects). All of the mean changes from baseline in vital signs were known pharmacologic effects of the treatments, and all ECGs with abnormal findings were considered not clinically significant. Neurocognitive scores were as expected with 1 outlier of mild cognitive impairment 1 hour after receiving MELT-100 6/50 mg that returned to normal mental functioning 2 hours postdose.

Although RSS scores of 2 at the 5-minute timepoint for MELT-100 at 3/25 mg and 6/50 mg sublingual doses were inconsistent with the median Tmax for each treatment, higher levels of sedation at the 30- and 35-minute timepoints (RSS scores of 2 through 4) were more consistent with the level of sedation expected at Tmax for MELT-100 sublingual tablets. This may be due to several potential factors.
(a) Because RSS scores at baseline were not assessed, and there was no impending surgical procedure increasing the potential to trigger symptoms of anxiety, it is unknown if this healthy subject population appeared cooperative, oriented, and tranquil (RSS score=2) at 5 minutes postdose as a result of their resting “calm” state or due to medication.
(b) Need for effective training on the use of RSS within the context of cataract surgery

Overall, MELT-100 at 3/25 mg and 6/50 mg single doses was well-tolerated when administered in healthy subjects under fasted conditions.

Midazolam Cmax, AUC0-t, and AUCinf were approximately 81%, 54%, and 54% lower, respectively, after MELT-100 sublingual tablet 3/25 mg (Treatment A, Test 1) compared to midazolam IV 3.5 mg (Treatment C, Reference 1). For 1-hydroxymidazolam, Cmax was approximately 37% higher and AUCs were approximately 12% to 15% lower after MELT-100 sublingual tablet 3/25 mg (Treatment A, Test 1) compared to midazolam IV 3.5 mg (Treatment C, Reference 1).

Midazolam Cmax was approximately 58% lower after MELT-100 sublingual tablet 6/50 mg (Treatment B, Test 2) compared to midazolam IV 3.5 mg (Treatment C, Reference 1); AUCs were similar for both treatments. For 1-hydroxymidazolam, Cmax, AUC0-t, and AUCinf were approximately 212%, 106%, and 90% higher, respectively, after MELT-100 sublingual tablet 6/50 mg (Treatment B, Test 2) compared to midazolam IV 3.5 mg (Treatment C, Reference 1).

Ketamine Cmax, AUC0-t, and AUCinf were approximately 86%, 75%, and 75% lower, respectively, after MELT-100 sublingual tablet 3/25 mg (Treatment A, Test 1) compared to ketamine IV 18 mg (Treatment D, Reference 2). For norketamine, Cmax, AUC0-t, and AUCinf were approximately 163%, 43%, and 26% higher, respectively, after MELT-100 sublingual tablet 3/25 mg (Treatment A, Test 1) compared to ketamine IV 18 mg (Treatment D, Reference 2).

Ketamine Cmax, AUC0-t, and AUCinf were approximately 72%, 45%, and 45% lower, respectively, after MELT-100 sublingual tablet 6/50 mg (Treatment B, Test 2) compared to ketamine IV 18 mg (Treatment D, Reference 2). For norketamine, Cmax, AUC0-t, and AUCinf were approximately 406%, 185%, and 149% higher, respectively, after MELT-100 sublingual tablet 6/50 mg (Treatment B, Test 2) compared to ketamine IV 18 mg (Treatment D, Reference 2).

After administrations of MELT-100 3/25 mg and MELT-100 6/50 mg, midazolam and 1-hydroxymidazolam exposure increased in a dose proportional manner with an increase in midazolam dose from 3 mg to 6 mg.

After administrations of MELT-100 3/25 mg and MELT-100 6/50 mg, ketamine and norketamine exposure increased in a dose proportional manner with an increase in ketamine dose from 25 mg to 50 mg.

Metabolite-to-parent ratios for midazolam and ketamine were higher after administration of MELT-100 than after IV administration, indicating some oral absorption of both drugs.

Exposures to midazolam and ketamine after administration of MELT-100 6/50 were less than or equal to those after administration of the IV reference standards, providing the necessary PK bridge.

TABLE 6
TABLE 6: PK Parameters of Midazolam after Administration of MELT 100 Sublingual Tablet
3/25 mg (Treatment A, Test 1), MELT 100 Sublingual Tablet 6/50 mg (Treatment B,
Test 2), and Midazolam IV 3.5 mg (Treatment C, Reference 1) - PK Population.

| | Treatment A: MELT-100 Sublingual Tablet 3/25 mg | | | | Treatment B: MELT-100 Sublingual Tablet 6/50 mg | | | |
|---|---|---|---|---|---|---|---|---|
| Parameter | n | Mean | SD | CV % | n | Mean | SD | CV % |
| Tₘₐₓ^{a} (h) | 23 | 0.500 (0.333-1.52) | | | 24 | 0.750 (0.500-1.50) | | |
| Cₘₐₓ (ng/mL) | 23 | 25.6 | 11.0 | 42.9 | 24 | 58.5 | 28.5 | 48.7 |
| AUC₀₋ₜ (h*ng/mL) | 23 | 79.5 | 29.1 | 36.6 | 24 | 172 | 61.5 | 35.7 |
| AUC_{inf} (h*ng/mL) | 23 | 85.1 | 33.0 | 38.8 | 24 | 184 | 73.2 | 39.7 |
| AUC_{Extrap} (%) | 23 | 5.82 | 3.30 | 56.8 | 24 | 5.56 | 3.96 | 71.2 |
| T_{1/2} (h) | 23 | 6.75 | 2.06 | 30.5 | 24 | 6.88 | 2.13 | 30.9 |
| Tₗₐₛₜ (h) | 23 | 22.1 | 3.78 | 17.1 | 24 | 23.4 | 2.27 | 9.73 |
| Cₗₐₛₜ (ng/mL) | 23 | 0.502 | 0.302 | 60.2 | 24 | 1.02 | 0.769 | 75.7 |
| CL/F (L/h) | 23 | 40.7 | 16.5 | 40.6 | 24 | 37.8 | 15.9 | 42.1 |
| V_{z}/F (L) | 23 | 364 | 104 | 28.7 | 24 | 344 | 88.4 | 25.7 |

| | Treatment C: Midazolam IV 3.5 mg | | | |
|---|---|---|---|---|
| Parameter | n | Mean | SD | CV % |
| Tₘₐₓ^{a} (h) | 24 | 0.183 (0.0833-1.00) | | |
| Cₘₐₓ (ng/mL) | 24 | 146 | 73.8 | 50.4 |
| AUC₀₋ₜ (h*ng/mL) | 24 | 168 | 38.7 | 22.9 |
| AUC_{inf} (h*ng/mL) | 24 | 179 | 44.7 | 24.9 |
| AUC_{Extrap} (%) | 24 | 5.58 | 3.75 | 67.2 |
| T_{1/2} (h) | 24 | 7.00 | 1.73 | 24.7 |
| Tₗₐₛₜ (h) | 24 | 23.4 | 2.27 | 9.71 |
| Cₗₐₛₜ (ng/mL) | 24 | 0.963 | 0.577 | 59.9 |
| CL (L/h) | 24 | 20.9 | 5.95 | 28.5 |
| V_{z} (L) | 24 | 202 | 48.3 | 23.9 |

^{a}Tₘₐₓ presented as median (range)
A = A single MELT-100 sublingual tablet (3 mg midazolam and 25 mg ketamine)
B = A single MELT-100 sublingual tablet (2 × 3 mg midazolam and 25 mg ketamine administered sublingually 15 min apart, total dose = 6/50 mg)
C = single dose of midazolam 3.5 mg administered as IV in 3 increments over 2 minutes of 1.5 mg, 1 mg, and 1 mg (2 minutes between each administration)

TABLE 7
PK Parameters of 1 hydroxymidazolam after Administration of MELT
100 Sublingual Tablet 3/25 mg (Treatment A, Test 1), MELT 100
Sublingual Tablet 6/50 mg (Treatment B, Test 2), and Midazolam
IV 3.5 mg (Treatment C, Reference 1) - PK Population.

| | Treatment A: | | | | Treatment B: | | | |
|---|---|---|---|---|---|---|---|---|
| | MELT-100 Sublingual | | | | MELT-100 Sublingual | | | |
| | Tablet 3/25 mg | | | | Tablet 6/50 mg | | | |
| Parameter | n | Mean | SD | CV % | n | Mean | SD | CV % |
| Tₘₐₓ^{a} (h) | 23 | 1.00 (0.350-4.00) | | | 24 | 0.900 (0.500-2.50) | | |
| Cₘₐₓ (ng/mL) | 23 | 7.78 | 5.02 | 64.4 | 24 | 16.2 | 6.28 | 38.6 |
| AUC₀₋ₜ (h*ng/mL) | 23 | 20.6 | 7.54 | 36.6 | 24 | 47.5 | 14.6 | 30.7 |
| AUC_{inf} (h*ng/mL) | 17 | 23.8 | 8.91 | 37.5 | 24 | 51.3 | 16.5 | 32.1 |
| AUC_{Extrap} (%) | 17 | 8.91 | 3.64 | 40.9 | 24 | 6.93 | 3.60 | 52.0 |
| T_{1/2} (h) | 17 | 5.87 | 3.44 | 58.6 | 24 | 6.84 | 2.89 | 42.2 |
| Tₗₐₛₜ (h) | 23 | 15.3 | 4.92 | 32.1 | 24 | 19.9 | 4.81 | 24.2 |
| Cₗₐₛₜ (ng/mL) | 23 | 0.265 | 0.0516 | 19.5 | 24 | 0.354 | 0.138 | 39.0 |
| MPR (AUC_{inf}) | 17 | 0.294 | 0.109 | 37.1 | 24 | 0.280 | 0.0754 | 27.0 |

| | Treatment C: | | | |
|---|---|---|---|---|
| | Midazolam IV 3.5 mg | | | |
| Parameter | n | Mean | SD | CV % |
| Tₘₐₓ^{a} (h) | 24 | 0.500 (0.333-2.00) | | |
| Cₘₐₓ (ng/mL) | 24 | 5.27 | 2.19 | 41.5 |
| AUC₀₋ₜ (h*ng/mL) | 24 | 22.7 | 6.55 | 28.8 |
| AUC_{inf} (h*ng/mL) | 21 | 26.8 | 7.99 | 29.8 |
| AUC_{Extrap} (%) | 21 | 14.1 | 5.15 | 36.6 |
| T_{1/2} (h) | 23 | 9.31 | 4.99 | 53.6 |
| Tₗₐₛₜ (h) | 24 | 19.0 | 4.47 | 23.5 |
| Cₗₐₛₜ (ng/mL) | 24 | 0.345 | 0.124 | 35.8 |
| MPR (AUC_{inf}) | 21 | 0.142 | 0.0294 | 20.7 |

^{a}Tₘₐₓ presented as median (range)
A = A single MELT-100 sublingual tablet (3 mg midazolam and 25 mg ketamine)
B = A single MELT-100 sublingual tablet (2 × 3 mg midazolam and 25 mg ketamine administered sublingually 15 min apart, total dose = 6/50 mg)
C = A single dose of midazolam 3.5 mg administered as IV in 3 increments over 2 minutes of 1.5 mg, 1 mg, and 1 mg (2 minutes between each administration)

TABLE 8
Statistical Analysis of the Natural Log-Transformed Systemic Exposure of Midazolam
and 1 hydroxymidazolam Comparing MELT 100 Sublingual Tablet 3/25 mg (Treatment
A, Test 1) to Midazolam IV 3.5 mg (Treatment C, Reference 1) - PK Population.

| | | | | | | | | |
|---|---|---|---|---|---|---|---|---|
| | | | GeoMean ^{a} | GeoMean ^{a} | Ratio | 90% | 90% | |
| Dependent | n | n | Test | Ref | (%)^{b} | CI ^{c} | CI ^{c} | ANOVA |
| Variable | (A) | (C) | (A) | (C) | (A/C) | Lower | Upper | CV % |

Midazolam

| | | | | | | | | |
|---|---|---|---|---|---|---|---|---|
| Cₘₐₓ | 23 | 24 | 23.8 | 127 | 18.70 | 15.53 | 22.50 | 38.67 |
| AUC₀₋ₜ | 23 | 24 | 75.1 | 164 | 45.68 | 42.65 | 48.91 | 13.83 |
| AUC_{inf} | 23 | 24 | 79.8 | 174 | 45.79 | 42.80 | 48.98 | 13.63 |

1-hydroxymidazolam

| | | | | | | | | |
|---|---|---|---|---|---|---|---|---|
| Cₘₐₓ | 23 | 24 | 6.73 | 4.91 | 137.15 | 115.61 | 162.70 | 35.48 |
| AUC₀₋ₜ | 23 | 24 | 19.4 | 22.0 | 88.06 | 82.64 | 93.83 | 12.83 |
| AUC_{inf} | 17 | 21 | 21.7 | 25.7 | 84.46 | 78.67 | 90.67 | 12.15 |

A = A single MELT-100 sublingual tablet (3 mg midazolam and 25 mg ketamine)
C = A single dose of midazolam 3.5 mg administered as IV in 3 increments over 2 minutes of 1.5 mg, 1 mg, and 1 mg (2 minutes between each administration)
Treatment A: (N = 23) Subject 219 was discontinued during Period 2 (Treatment A) and Subject 205 was discontinued during Period 4 (Treatment A)
Treatment C: (N = 24) Subject 219 was discontinued during Period 2 and did not receive Treatment C
^{a} Geometric Mean based on Least Squares Mean of log-transformed parameter values
^{b}Ratio (%) = Geometric Mean (A)/Geometric Mean (C)
^{c} 90% Confidence Interval

TABLE 9
Statistical Analysis of the Natural Log-Transformed Systemic Exposure of Midazolam
and 1 hydroxymidazolam Comparing MELT 100 Sublingual Tablet 6/50 mg (Treatment
B, Test 2) to Midazolam IV 3.5 mg (Treatment C, Reference 1) - PK Population.

| | | | | | | | | |
|---|---|---|---|---|---|---|---|---|
| | | | GeoMean ^{a} | GeoMean ^{a} | Ratio | 90% | 90% | |
| Dependent | n | n | Test | Ref | (%)^{b} | CI ^{c} | CI ^{c} | ANOVA |
| Variable | (B) | (C) | (B) | (C) | (B/C) | Lower | Upper | CV % |

Midazolam

| | | | | | | | | |
|---|---|---|---|---|---|---|---|---|
| Cₘₐₓ | 24 | 24 | 53.8 | 127 | 42.35 | 35.31 | 50.79 | 38.67 |
| AUC₀₋ₜ | 24 | 24 | 162 | 164 | 98.44 | 92.06 | 105.27 | 13.83 |
| AUC_{inf} | 24 | 24 | 172 | 174 | 98.46 | 92.17 | 105.18 | 13.63 |

1-hydroxymidazolam

| | | | | | | | | |
|---|---|---|---|---|---|---|---|---|
| Cₘₐₓ | 24 | 24 | 15.3 | 4.91 | 311.82 | 263.69 | 368.74 | 35.48 |
| AUC₀₋ₜ | 24 | 24 | 45.4 | 22.0 | 206.45 | 193.99 | 219.70 | 12.83 |
| AUC_{inf} | 24 | 21 | 48.8 | 25.7 | 190.17 | 178.64 | 202.45 | 12.15 |

B = A single MELT-100 sublingual tablet (2 × 3 mg midazolam and 25 mg ketamine administered sublingually 15 min apart, total dose = 6/50 mg)
C = A single dose of midazolam 3.5 mg administered as IV in 3 increments over 2 minutes of 1.5 mg, 1 mg, and 1 mg (2 minutes between each administration)
Treatment B and C: (N = 24) Subject 219 was discontinued during Period 2 and did not receive Treatments B and C
^{a} Geometric Mean based on Least Squares Mean of log-transformed parameter values
^{b}Ratio (%) = Geometric Mean (B)/Geometric Mean (C)
^{c} 90% Confidence Interval

TABLE 10
PK Parameters of Ketamine after Administration of MELT 100 Sublingual Tablet
3/25 mg (Treatment A, Test 1), MELT 100 Sublingual Tablet 6/50 mg (Treatment
B, Test 2), and Ketamine IV 18 mg (Treatment D, Reference 2) - PK Population.

| | Treatment A: | | | | Treatment B: | | | |
|---|---|---|---|---|---|---|---|---|
| | MELT-100 Sublingual | | | | MELT-100 Sublingual | | | |
| | Tablet 3/25 mg | | | | Tablet 6/50 mg | | | |
| Parameter | n | Mean | SD | CV % | n | Mean | SD | CV % |
| Tₘₐₓ^{a} (h) | 23 | 0.500 (0.250-1.35) | | | 24 | 0.517 (0.500-1.25) | | |
| Cₘₐₓ (ng/mL) | 23 | 31.2 | 11.5 | 36.8 | 24 | 63.4 | 24.2 | 38.2 |
| AUC₀₋ₜ (h*ng/mL) | 23 | 77.1 | 34.0 | 44.2 | 24 | 163 | 61.0 | 37.4 |
| AUC_{inf} (h*ng/mL) | 19 | 86.3 | 41.5 | 48.1 | 21 | 184 | 74.7 | 40.5 |
| AUC_{Extrap} (%) | 19 | 10.2 | 3.47 | 33.9 | 21 | 9.78 | 4.18 | 42.8 |
| T_{1/2} (h) | 19 | 5.22 | 3.58 | 68.6 | 21 | 8.59 | 4.23 | 49.3 |
| Tₗₐₛₜ (h) | 23 | 12.5 | 4.88 | 38.9 | 24 | 18.9 | 5.23 | 27.7 |
| Cₗₐₛₜ (ng/mL) | 23 | 1.23 | 0.163 | 13.2 | 24 | 1.48 | 0.357 | 24.2 |
| CL/F (L/h) | 19 | 350 | 147 | 41.8 | 21 | 315 | 126 | 40.1 |
| V_{z}/F (L) | 19 | 2110 | 766 | 36.4 | 21 | 3400 | 1320 | 38.8 |

| | Treatment D: | | | |
|---|---|---|---|---|
| | Ketamine IV 18 mg | | | |
| Parameter | n | Mean | SD | CV % |
| Tₘₐₓ^{a} (h) | 25 | 0.167 (0.0833-0.267) | | |
| Cₘₐₓ (ng/mL) | 25 | 250 | 187 | 74.8 |
| AUC₀₋ₜ (h*ng/mL) | 25 | 283 | 63.9 | 22.5 |
| AUC_{inf} (h*ng/mL) | 24 | 316 | 75.5 | 23.9 |
| AUC_{Extrap} (%) | 24 | 9.64 | 4.24 | 44.0 |
| T_{1/2} (h) | 24 | 10.3 | 2.79 | 27.2 |
| Tₗₐₛₜ (h) | 25 | 23.7 | 1.61 | 6.78 |
| Cₗₐₛₜ (ng/mL) | 25 | 1.99 | 0.678 | 34.1 |
| CL (L/h) | 24 | 59.9 | 14.2 | 23.6 |
| V_{z} (L) | 24 | 867 | 240 | 27.7 |

^{a}Tₘₐₓ presented as median (range)
A = A single MELT-100 sublingual tablet (3 mg midazolam and 25 mg ketamine)
B = A single MELT-100 sublingual tablet (2 × 3 mg midazolam and 25 mg ketamine administered sublingually 15 min apart, total dose = 6/50 mg)
D = A single dose of ketamine 18 mg (10 mg/mL in a 20 mL vial) administered as IV over 1 minute

TABLE 11
PK Parameters of Norketamine after Administration of MELT 100 Sublingual Tablet
3/25 mg (Treatment A, Test 1), MELT 100 Sublingual Tablet 6/50 mg (Treatment
B, Test 2), and Ketamine IV 18 mg (Treatment D, Reference 2) - PK Population.

| | Treatment A: | | | | Treatment B: | | | |
|---|---|---|---|---|---|---|---|---|
| | MELT-100 Sublingual | | | | MELT-100 Sublingual | | | |
| | Tablet 3/25 mg | | | | Tablet 6/50 mg | | | |
| Parameter | n | Mean | SD | CV % | n | Mean | SD | CV % |
| Tₘₐₓ^{a} (h) | 23 | 1.25 (0.500-4.00) | | | 24 | 1.13 (0.500-2.50) | | |
| Cₘₐₓ (ng/mL) | 23 | 102 | 34.0 | 33.4 | 24 | 193 | 51.6 | 26.8 |
| AUC₀₋ₜ (h*ng/mL) | 23 | 469 | 131 | 28.0 | 24 | 935 | 259 | 27.7 |
| AUC_{inf} (h*ng/mL) | 22 | 561 | 189 | 33.8 | 23 | 1070 | 296 | 27.7 |
| AUC_{Extrap} (%) | 22 | 14.9 | 6.17 | 41.5 | 23 | 13.9 | 5.71 | 41.1 |
| T_{1/2} (h) | 23 | 10.5 | 3.15 | 29.9 | 24 | 10.2 | 3.04 | 29.7 |
| Tₗₐₛₜ (h) | 23 | 23.7 | 1.67 | 7.06 | 24 | 23.7 | 1.65 | 6.94 |
| Cₗₐₛₜ (ng/mL) | 23 | 5.81 | 2.72 | 46.8 | 24 | 11.0 | 5.20 | 47.2 |
| MPR (AUC_{inf}) | 18 | 7.06 | 1.84 | 26.1 | 20 | 6.74 | 1.46 | 21.7 |

| | Treatment D: | | | |
|---|---|---|---|---|
| | Ketamine IV 18 mg | | | |
| Parameter | n | Mean | SD | CV % |
| Tₘₐₓ^{a} (h) | 24 | 0.775 (0.333-2.50) | | |
| Cₘₐₓ (ng/mL) | 24 | 38.3 | 11.0 | 28.7 |
| AUC₀₋ₜ (h*ng/mL) | 24 | 324 | 78.7 | 24.3 |
| AUC_{inf} (h*ng/mL) | 18 | 437 | 121 | 27.8 |
| AUC_{Extrap} (%) | 18 | 21.1 | 5.78 | 27.4 |
| T_{1/2} (h) | 24 | 13.8 | 5.21 | 37.7 |
| Tₗₐₛₜ (h) | 24 | 24.0 | 0.0752 | 0.313 |
| Cₗₐₛₜ (ng/mL) | 24 | 5.81 | 1.79 | 30.8 |
| MPR (AUC_{inf}) | 17 | 1.50 | 0.310 | 20.6 |

^{a}Tₘₐₓ presented as median (range)
A = A single MELT-100 sublingual tablet (3 mg midazolam and 25 mg ketamine)
B = A single MELT-100 sublingual tablet (2 × 3 mg midazolam and 25 mg ketamine administered sublingually 15 min apart, total dose = 6/50 mg)
D = A single dose of ketamine 18 mg (10 mg/mL in a 20 mL vial) administered as IV over 1 minute

TABLE 12
Statistical Analysis of the Natural Log-Transformed Systemic Exposure of
Ketamine and Nor-ketamine Comparing MELT 100 Sublingual Tablet 3/25 mg
(Treatment A, Test 1) to Ketamine IV 18 mg (Treatment D, Reference 2)

| | | | | | | | | |
|---|---|---|---|---|---|---|---|---|
| | | | GeoMean ^{a} | GeoMean ^{a} | Ratio | 90% | 90% | |
| Dependent | n | n | Test | Ref | (%)^{b} | CI ^{c} | CI ^{c} | ANOVA |
| Variable | (A) | (D) | (A) | (D) | (A/D) | Lower | Upper | CV % |

Ketamine

| | | | | | | | | |
|---|---|---|---|---|---|---|---|---|
| Cₘₐₓ | 23 | 25 | 29.4 | 208 | 14.15 | 11.36 | 17.61 | 46.61 |
| AUC₀₋ₜ | 23 | 25 | 70.3 | 277 | 25.36 | 22.72 | 28.31 | 22.46 |
| AUC_{inf} | 19 | 24 | 78.3 | 307 | 25.46 | 22.38 | 28.98 | 24.34 |

Norketamine

| | | | | | | | | |
|---|---|---|---|---|---|---|---|---|
| Cₘₐₓ | 23 | 24 | 97.5 | 37.0 | 263.27 | 233.91 | 296.30 | 23.95 |
| AUC₀₋ₜ | 23 | 24 | 450 | 316 | 142.75 | 136.86 | 148.90 | 8.41 |
| AUC_{inf} | 22 | 18 | 532 | 422 | 126.04 | 120.15 | 132.21 | 8.28 |

A = A single MELT-100 sublingual tablet (3 mg midazolam and 25 mg ketamine)
D = A single dose of ketamine 18 mg (10 mg/mL in a 20 mL vial) administered as IV over 1 minute
Treatment A: (N = 23) Subject 219 was discontinued during Period 2 (Treatment A) and Subject 205 was discontinued during Period 4 (Treatment A)
For norketamine (Treatment D; N = 24) Subject 218 was excluded from the Treatment A vs. Treatment D comparison due to a quantifiable predose >5% Cₘₐₓ
^{a} Geometric Mean based on Least Squares Mean of log-transformed parameter values
^{b}Ratio (%) = Geometric Mean (A)/Geometric Mean (D)
^{c} 90% Confidence Interval

TABLE 13
Statistical Analysis of the Natural Log-Transformed Systemic Exposure of
Ketamine and Norketamine Comparing MELT-100 Sublingual Tablet 6/50 mg
(Treatment B, Test 2) to Ketamine IV 18 mg (Treatment D, Reference 2)

| | | | | | | | | |
|---|---|---|---|---|---|---|---|---|
| | | | GeoMean ^{a} | GeoMean ^{a} | Ratio | 90% | 90% | |
| Dependent | n | n | Test | Ref | (%)^{b} | CI ^{c} | CI ^{c} | ANOVA |
| Variable | (B) | (D) | (B) | (D) | (B/D) | Lower | Upper | CV % |

Ketamine

| | | | | | | | | |
|---|---|---|---|---|---|---|---|---|
| Cₘₐₓ | 24 | 25 | 59.1 | 208 | 28.46 | 22.92 | 35.33 | 46.61 |
| AUC₀₋ₜ | 24 | 25 | 153 | 277 | 55.04 | 49.39 | 61.33 | 22.46 |
| AUC_{inf} | 21 | 24 | 170 | 307 | 55.36 | 48.94 | 62.63 | 24.34 |

Norketamine

| | | | | | | | | |
|---|---|---|---|---|---|---|---|---|
| Cₘₐₓ | 24 | 24 | 187 | 37.0 | 505.57 | 449.90 | 568.12 | 23.95 |
| AUC₀₋ₜ | 24 | 24 | 901 | 316 | 285.42 | 273.81 | 297.52 | 8.41 |
| AUC_{inf} | 23 | 18 | 1050 | 422 | 249.20 | 237.75 | 261.21 | 8.28 |

B = A single MELT-100 sublingual tablet (2 × 3 mg midazolam and 25 mg ketamine administered sublingually 15 min apart, total dose = 6/50 mg)
D = A single dose of ketamine 18 mg (10 mg/mL in a 20 mL vial) administered as IV over 1 minute
Treatment B: (N = 24) Subject 219 was discontinued in Period 2 and did not receive Treatment B
For norketamine: (Treatment D: N = 24) Subject 218 was excluded due to a quantifiable predose >5% Cₘₐₓ in Treatment D
^{a} Geometric Mean based on Least Squares Mean of log-transformed parameter values
^{b}Ratio (%) = Geometric Mean (B)/Geometric Mean (D)
^{c} 90% Confidence Interval

TABLE 14
Statistical Analysis of the Natural Log-Transformed Systemic Exposure of Midazolam
and 1-hydroxymidazolam Comparing MELT-100 Sublingual Tablet 6/50 mg (Treatment
B, Test 2) to MELT-100 Sublingual Tablet 3/25 mg (Treatment A, Test 1)

| | | | | | | | | |
|---|---|---|---|---|---|---|---|---|
| | | | GeoMean ^{a} | GeoMean ^{a} | Ratio | 90% | 90% | |
| Dependent | n | n | Test | Ref | (%)^{b} | CI ^{c} | CI ^{c} | ANOVA |
| Variable | (B) | (A) | (B) | (A) | (B/A) | Lower | Upper | CV % |

Midazolam

| | | | | | | | | |
|---|---|---|---|---|---|---|---|---|
| Cₘₐₓ | 24 | 23 | 53.4 | 24.1 | 222.06 | 197.49 | 249.68 | 23.15 |
| AUC₀₋ₜ | 24 | 23 | 162 | 75.4 | 214.72 | 204.51 | 225.45 | 9.50 |
| AUC_{inf} | 24 | 23 | 172 | 80.1 | 214.12 | 203.65 | 225.12 | 9.76 |

1-hydroxymidazolam

| | | | | | | | | |
|---|---|---|---|---|---|---|---|---|
| Cₘₐₓ | 24 | 23 | 15.2 | 6.83 | 222.90 | 187.11 | 265.54 | 35.22 |
| AUC₀₋ₜ | 24 | 23 | 45.4 | 19.5 | 232.68 | 220.24 | 245.83 | 10.72 |
| AUC_{inf} | 24 | 17 | 48.8 | 21.6 | 226.20 | 213.59 | 239.55 | 9.23 |

B = A single MELT-100 sublingual tablet (2 × 3 mg midazolam and 25 mg ketamine administered sublingually 15 min apart, total dose = 6/50 mg)
A = A single MELT-100 sublingual tablet (3 mg midazolam and 25 mg ketamine)
Treatment A: (N = 23) Subject 219 was discontinued during Period 2 (Treatment A) and Subject 205 was discontinued during Period 4 (Treatment A)
Treatment B: (N = 24) Subject 219 was discontinued during Period 2 and did not receive Treatment B
^{a} Geometric Mean based on Least Squares Mean of log-transformed parameter values
^{b}Ratio (%) = Geometric Mean (B)/Geometric Mean (A)
^{c} 90% Confidence Interval

TABLE 15
Statistical Analysis of the Natural Log-Transformed Systemic Exposure of Ketamine
and Nor-ketamine Comparing MELT 100 Sublingual Tablet 6/50 mg (Treatment
B, Test 2) to MELT 100 Sublingual Tablet 3/25 mg (Treatment A, Test 1)

| | | | | | | | | |
|---|---|---|---|---|---|---|---|---|
| | | | GeoMean ^{a} | GeoMean ^{a} | Ratio | 90% | 90% | |
| Dependent | n | n | Test | Ref | (%)^{b} | CI ^{c} | CI ^{c} | ANOVA |
| Variable | (B) | (A) | (B) | (A) | (B/A) | Lower | Upper | CV % |

Ketamine

| | | | | | | | | |
|---|---|---|---|---|---|---|---|---|
| Cₘₐₓ | 24 | 23 | 59.0 | 29.9 | 197.72 | 178.74 | 218.72 | 19.85 |
| AUC₀₋ₜ | 24 | 23 | 152 | 70.8 | 215.09 | 199.14 | 232.31 | 15.07 |
| AUC_{inf} | 21 | 19 | 166 | 79.4 | 208.79 | 190.18 | 229.21 | 14.11 |

Norketamine

| | | | | | | | | |
|---|---|---|---|---|---|---|---|---|
| Cₘₐₓ | 24 | 23 | 187 | 98.4 | 190.10 | 174.27 | 207.37 | 17.07 |
| AUC₀₋ₜ | 24 | 23 | 901 | 450 | 200.25 | 193.07 | 207.70 | 7.11 |
| AUC_{inf} | 23 | 22 | 1050 | 533 | 197.84 | 189.40 | 206.65 | 8.12 |

B = A single MELT-100 sublingual tablet (2 × 3 mg midazolam and 25 mg ketamine administered sublingually 15 min apart, total dose = 6/50 mg)
A = A single MELT-100 sublingual tablet (3 mg midazolam and 25 mg ketamine)
^{a} Geometric Mean based on Least Squares Mean of log-transformed parameter values
Treatment A: (N = 23) Subject 219 was discontinued during Period 2 (Treatment A) and Subject 205 was discontinued during Period 4 (Treatment A)
Treatment B: (N = 24) Subject 219 was discontinued during Period 2 and did not receive Treatment B
^{b}Ratio (%) = Geometric Mean (B)/Geometric Mean (A)
^{c} 90% Confidence Interval

### Example 2: MELT-300 Sublingual Tablet Phase II Efficacy and Safety Study

### Introduction

A Phase 2 efficacy and safety study of the MELT-300 formulation was conducted at nine sites with over 300 enrolled subjects. MELT-300 is a sublingual, needle- and opioid-free formulation for procedural sedation during cataract surgery. MELT-300 combines fixed doses of midazolam (3 mg) and ketamine (50 mg) in one rapidly dissolving tablet (RDT) that is administered sublingually for procedural sedation (e.g., during cataract surgery). MELT-300 utilizes fast-dissolving delivery technology (Zydis®, Catalent Inc.) to rapidly dissolve the tablet for absorption across the sublingual mucosa.

The Phase 2 study was factorial-designed, randomized, double-blind, placebo-controlled, and included parallel-cohorts. Study design emphasized evaluation of efficacy and safety of MELT-300, and contribution of the midazolam and ketamine components towards sedation and intraoperative ocular analgesia in subjects undergoing cataract extraction with lens replacement (CELR). Comparisons were made of MELT-300 administration against: (i) placebo alone; (ii) sublingually delivered midazolam alone; and (iii) sublingually delivered ketamine alone. Primary efficacy endpoints included appropriate sedation during the cataract surgery and management of intraoperative pain during the surgery.

The formulation of the MELT-300 sublingual tablet is provided below in Table 16.

TABLE 16
MELT-300 Formulation

| | | | |
|---|---|---|---|
| | MELT-300 | | Reference to |
| Ingredient | Quantity per tablet (mg) | Function | standards |

Active ingredients

| | | | |
|---|---|---|---|
| Midazolam | 3.00 | Active ingredient | USP |
| Ketamine HCl | 50.00 | Active ingredient | USP |

Excipients

| | | | |
|---|---|---|---|
| Gelatin ⁽¹⁾ | 27.60 | Matrix former | NF |
| Mannitol | 21.00 | Matrix former | USP |
| Sucralose micronized | 1.26 | Sweetener | NF |
| Orange flavor 501701 AP0551 | 2.52 | Flavor | |
| Citric acid | qs pH 3.45 to 3.75 | pH adjuster | USP |
| Purified water ⁽²⁾ | qs 600.00 | Dispersing vehicle | USP |

Processing aids

| | | | |
|---|---|---|---|
| Liquid nitrogen | N/A | Freezing | USP |

Totals

| | | | |
|---|---|---|---|
| Total wet dosing weight | 600.00 | N/A | N/A |

⁽¹⁾ Bovine gelatin.
⁽²⁾ Not present in the finished product, removed during processing.
HCl: hydrochloride;
USP: United States Pharmacopoeia;
NF: National Formulary;
qs = quantum satis;
N/A: not applicable

### Results:

Overall disposition and subject accountability of a randomized subjects analysis set is provided in TABLE 17. Total subjects included in the randomized subjects analysis was N=338, with treatment arms of MELT-300 (N=86), midazolam (N=87), ketamine (N=85), and placebo (N=80).

The overall disposition and subject accountability of the full analysis set is provided in TABLE 18. Total subjects included in the full analysis was N=310, with treatment arms of MELT-300 (N=77), midazolam (N=81), ketamine (N=79), and placebo (N=73).

Demographics and subject baseline characteristics of the full analysis set are provided in TABLE 19. Hospital Anxiety and Depression Scale (HADS) and chronic pain ratings (e.g., severe pain, moderate pain, mild pain, or no pain) were collected from each subject. Preoperative intraocular pressure in each subject's study and non-study eye was also measured.

Achievement of procedural sedation was evaluated for each subject (see TABLE 20). Success for procedural sedation for this Phase 2 study was defined as achieving target sedation level (Ramsay Sedation Scale (RSS) scale level 2 or 3) at the start of surgery without the need for rescue sedation medication, and no need for intraoperative rescue sedation medication to maintain target sedation level.

63/77 (81.8%) subjects receiving MELT-300 achieved target sedation, compared to 52/81 (64.2%) subjects receiving 3 mg midazolam alone, 50/79 (63.3%) subjects receiving 50 mg ketamine alone, and 26/73 (35.6%) of subjects receiving placebo. p-Values between the MELT-300 treatment arm and the midazolam, ketamine, or placebo treatment arms were p=0.0129. p=0.0096, and p<0.0001, respectively.

Mean intraoperative analgesia by the numeric pain rating scale (Min=0; Max=9) was assayed for each subject in the full analysis set at intraoperative timepoints: insertion of the speculum; corneal incision; insertion of phacoemulsion probe; insertion of the synthetic replacement lens. An average of the available timepoints was also calculated. TABLE 21 provides results for mean intraoperative analgesia by the numeric pain rating scale (carry forward). TABLE 22 provides results for mean intraoperative analgesia by the numeric pain rating scale, adjusted for site (carry forward). TABLE 23 provides results for mean intraoperative analgesia by the numeric pain rating scale (observed). Statistical analyses (e.g., least squares mean and difference. 95% confidence interval, and p-values) between treatment arms of MELT-300, midazolam alone, ketamine alone, and placebo are provided in TABLES 5-7.

Subject-rated worst pain during surgery by the numeric pain rating scale (Min=0; Max=9) was assayed for each subject in the full analysis set, which is provided in TABLE 24. Statistical analyses (e.g., mean difference, 95% confidence interval, and p-values) between treatment arms of MELT-300, midazolam alone, ketamine alone, and placebo are provided.

The number of subjects achieving analgesia response (observed) in the full analysis set was determined. Success in analgesia response was defined as NPRS<4 at the available at the available four intraoperative timepoints and a failure otherwise (e.g., any interoperative pain rescue was assumed to be a failure. Results are provided in TABLE 25. The number of subjects completing the surgery is provided in TABLE 26, and the number of subjects completing the surgery without interruption is provided in TABLE 27. For this measure, subjects completing the surgery without interruption (other than rescue medication) due to pain or anxiety were considered successes. The number of subjects requiring rescue sedative medication and those requiring analgesic medication are provided in TABLES 28 and 29, respectively. For all TABLES 25-29, p-Values between pairs of treatments were calculated and are provided.

Post-operation, subjects were asked whether they would want the study drug again for a second surgery using an 11-point Likert scale of 0 (not likely at all) to 10 (extremely likely). Results are provided in TABLE 30. Statistical analyses (e.g., mean difference, 95% confidence interval, and p-values) between treatment arms of MELT-300, midazolam alone, ketamine alone, and placebo are provided.

The number of subjects receiving opioid concomitant medication postoperatively is provided in TABLE 31. Total dose is the sum of all opioids taken in milligrams for each subject in IV morphine equivalent.

Treatment-emergent ocular adverse events (TEAEs) by system, organ class, and preferred term are provided for the full analysis set. The study eye (TABLE 32) non-study eye (TABLE 33), or either eye (TABLE 34) was assessed. TEAES were defined as events that started on or after the date of the study drug administration through the end of Visit 4 or early withdrawal. Subjects with one or more adverse event within a level of Medical Dictionary for Regulatory Activities (MedDRA) were counted only once in that level. For the study eye, overall 2/85 (2.4%) of subjects in the MELT-300 treatment arm experienced a treatment-emergent ocular adverse event, compared to 7/87 (8.0%) for midazolam alone, 6/85 (7.1%) for ketamine alone, and 12/79 (15.2%) for placebo. TEAEs of special interest by system, organ class, and preferred term are provided in TABLE 35 for the full analysis set.

TABLE 17
Overall Disposition and Subject Accountability (Analysis Set: Randomized Subjects).

| | | MELT-300 | | Midazolam 3 mg | | Ketamine 50 mg | | Placebo | | Overall | |
|---|---|---|---|---|---|---|---|---|---|---|---|
| | Statistic | (N = 86) | | (N = 87) | | (N = 85) | | (N = 80) | | (N = 338) | |
| Screened | n | | | | | | | | | 490 | |
| Randomized | n (%) | 86 | (100.0) | 87 | (100.0) | 85 | (100.0) | 80 | (100.0) | 338 | (100.0) |
| Randomized with Any Protocol Deviation | n (%) | 42 | (48.8) | 40 | (46.0) | 45 | (52.9) | 46 | (57.5) | 173 | (51.2) |
| Treated | n (%) | 85 | (98.8) | 87 | (100.0) | 85 | (100.0) | 79 | (98.8) | 336 | (99.4) |
| Treated with Any Protocol Deviation | n (%) | 42 | (48.8) | 40 | (46.0) | 45 | (52.9) | 46 | (57.5) | 173 | (51.2) |
| Safety Analysis Set | n (%) | 85 | (98.8) | 87 | (100.0) | 85 | (100.0) | 79 | (98.8) | 336 | (99.4) |
| Full Analysis Set | n (%) | 77 | (89.5) | 81 | (93.1) | 79 | (92.9) | 73 | (91.3) | 310 | (91.7) |
| Per-Protocol Analysis Set | n (%) | 72 | (83.7) | 72 | (82.8) | 75 | (88.2) | 69 | (86.3) | 288 | (85.2) |
| Completed Study | n (%) | | | | | | | | | | |
| Yes | | 85 | (98.8) | 87 | (100.0) | 85 | (100.0) | 78 | (97.5) | 335 | (99.1) |
| No | | 1 | (1.2) | 0 | (0) | 0 | (0) | 2 | (2.5) | 3 | (0.9) |
| Reason for Discontinuation of Study | n (%) | | | | | | | | | | |
| Withdrawal by Subject | | 1 | (100.0) | 0 | (0) | 0 | (0) | 1 | (50.0) | 2 | (56.7) |
| Adverse Event | | 0 | (0) | 0 | (0) | 0 | (0) | 0 | (0) | 0 | (0) |
| Pregnancy | | 0 | (0) | 0 | (0) | 0 | (0) | 0 | (0) | 0 | (0) |
| Physician Decision | | 0 | (0) | 0 | (0) | 0 | (0) | 0 | (0) | 0 | (0) |
| Protocol Violation | | 0 | (0) | 0 | (0) | 0 | (0) | 1 | (50.0) | 1 | (33.3) |
| Lost to Follow-Up | | 0 | (0) | 0 | (0) | 0 | (0) | 0 | (0) | 0 | (0) |
| Other | | 0 | (0) | 0 | (0) | 0 | (0) | 0 | (0) | 0 | (0) |
| Visit Attendance | n (%) | | | | | | | | | | |
| Visit 1 | | 86 | (100.0) | 87 | (100.0) | 85 | (100.0) | 80 | (100.0) | 338 | (100.0) |
| Visit 2 (Day 1 Pre-Op) | | 86 | (100.0) | 87 | (100.0) | 85 | (100.0) | 80 | (100.0) | 338 | (100.0) |
| Visit 2 (Day 1 Surgery) | | 85 | (98.8) | 87 | (100.0) | 85 | (100.0) | 79 | (98.8) | 336 | (99.4) |
| Visit 2 (Day 1 Post-Op) | | 85 | (98.8) | 87 | (100.0) | 85 | (100.0) | 79 | (98.8) | 336 | (99.4) |
| Visit 2 (Day 1 6 hr Phone Call) | | 85 | (98.8) | 87 | (100.0) | 84 | (98.8) | 76 | (95.0) | 332 | (98.2) |
| Visit 3 (Day 2 Visit or Phone Call) | | 85 | (98.8) | 87 | (100.0) | 84 | (98.8) | 78 | (97.5) | 334 | (98.8) |
| Visit 4 (Day 8) | | 85 | (98.8) | 87 | (100.0) | 85 | (100.0) | 78 | (97.5) | 335 | (99.1) |

Note:
Percentages are based on the number of subjects randomized in each treatment group or overall. For reason for discontinuation from study, percentages are based on the number of subjects who discontinued the study.

TABLE 18
Overall Disposition and Subject Accountability (Analysis Set: Full Analysis Set)

| | | MELT-300 | | Midazolam 3 mg | | Ketamine 50 mg | | Placebo | | Overall | |
|---|---|---|---|---|---|---|---|---|---|---|---|
| | Statistic | (N = 77) | | (N = 81) | | (N = 79) | | (N = 73) | | (N = 310) | |
| Randomized | n (%) | 77 | (100.0) | 81 | (100.0) | 79 | (100.0) | 73 | (100.0) | 310 | (100.0) |
| Randomized with Any Protocol Deviation | n (%) | 35 | (45.5) | 35 | (43.2) | 40 | (50.6) | 40 | (54.8) | 150 | (48.4) |
| Treated | n (%) | 77 | (100.0) | 81 | (100.0) | 79 | (100.0) | 73 | (100.0) | 310 | (100.0) |
| Treated with Any Protocol Deviation | n (%) | 35 | (45.5) | 35 | (43.2) | 40 | (50.6) | 40 | (54.8) | 150 | (48.4) |
| Completed Study | n (%) | | | | | | | | | | |
| Yes | | 77 | (100.0) | 81 | (100.0) | 79 | (100.0) | 73 | (100.0) | 310 | (100.0) |
| No | | 0 | (0) | 0 | (0) | 0 | (0) | 0 | (0) | 0 | (0) |
| Reason for Discontinuation of Study | n (%) | | | | | | | | | | |
| Withdrawal by Subject | | 0 | (0) | 0 | (0) | 0 | (0) | 0 | (0) | 0 | (0) |
| Adverse Event | | 0 | (0) | 0 | (0) | 0 | (0) | 0 | (0) | 0 | (0) |
| Pregnancy | | 0 | (0) | 0 | (0) | 0 | (0) | 0 | (0) | 0 | (0) |
| Physician Decision | | 0 | (0) | 0 | (0) | 0 | (0) | 0 | (0) | 0 | (0) |
| Protocol Violation | | 0 | (0) | 0 | (0) | 0 | (0) | 0 | (0) | 0 | (0) |
| Lost to Follow-Up | | 0 | (0) | 0 | (0) | 0 | (0) | 0 | (0) | 0 | (0) |
| Other | | 0 | (0) | 0 | (0) | 0 | (0) | 0 | (0) | 0 | (0) |
| Visit Attendance | n (%) | | | | | | | | | | |
| Visit 1 | | 77 | (100.0) | 81 | (100.0) | 79 | (100.0) | 73 | (100.0) | 310 | (100.0) |
| Visit 2 (Day 1 Pre-Op) | | 77 | (100.0) | 81 | (100.0) | 79 | (100.0) | 73 | (100.0) | 310 | (100.0) |
| Visit 2 (Day 1 Surgery) | | 77 | (100.0) | 81 | (100.0) | 79 | (100.0) | 73 | (100.0) | 310 | (100.0) |
| Visit 2 (Day 1 Post-Op) | | 77 | (100.0) | 81 | (100.0) | 79 | (100.0) | 73 | (100.0) | 310 | (100.0) |
| Visit 2 (Day 1 6 hr Phone Call) | | 77 | (100.0) | 81 | (100.0) | 78 | (98.7) | 70 | (95.9) | 305 | (98.7) |
| Visit 3 (Day 2 Visit or Phone Call) | | 77 | (100.0) | 81 | (100.0) | 78 | (98.7) | 72 | (98.6) | 308 | (99.4) |
| Visit 4 (Day 8) | | 77 | (100.0) | 81 | (100.0) | 79 | (100.0) | 73 | (100.0) | 310 | (100.0) |

Note:
Percentages are based on the number of subjects randomized in each treatment group or overall. For reason for discontinuation from study, percentages are based on the number of subjects who discontinued the study.

TABLE 19
Demographic and Baseline Characteristics (Analysis Set: Full Analysis Set)

| | | MELT-300 | | Midazolam 3 mg | | Ketamine 50 mg | | Placebo | | Overall | |
|---|---|---|---|---|---|---|---|---|---|---|---|
| | Statistic | (N = 77) | | (N = 81) | | (N = 79) | | (N = 73) | | (N = 310) | |
| Age (years) | n (missing) | 77 | (0) | 81 | (0) | 79 | (0) | 73 | (0) | 310 | (0) |
| | Mean (SD) | 68.9 | (7.4) | 69.1 | (7.9) | 70.4 | (7.4) | 68.7 | (6.4) | 69.3 | (7.3) |
| | Median | 68.0 | | 69.0 | | 70.0 | | 68.0 | | 69.0 | |
| | Min; Max | 56; 87 | | 50; 91 | | 55; 93 | | 52; 83 | | 50; 93 | |
| Age Categories | n (missing) | 77 | (0) | 81 | (0) | 79 | (0) | 73 | (0) | 310 | (0) |
| <65 years | n (%) | 18 | (23.4) | 25 | (30.9) | 17 | (21.5) | 17 | (23.3) | 77 | (24.8) |
| >=65 years | | 59 | (76.6) | 56 | (69.1) | 62 | (78.5) | 56 | (76.7) | 233 | (75.2) |
| Sex | n (missing) | 77 | (0) | 81 | (0) | 79 | (0) | 73 | (0) | 310 | (0) |
| Malo | n (%) | 35 | (45.5) | 39 | (48.1) | 42 | (53.2) | 42 | (57.5) | 158 | (51.0) |
| Female | | 42 | (54.5) | 42 | (51.9) | 37 | (46.8) | 31 | (42.5) | 152 | (49.0) |
| Race | n (missing) | 77 | (0) | 81 | (0) | 79 | (0) | 73 | (0) | 310 | (0) |
| White | n (%) | 59 | (76.6) | 66 | (81.5) | 64 | (81.0) | 64 | (87.7) | 253 | (81.6) |
| Black or African American | | 18 | (23.4) | 15 | (18.5) | 14 | (17.7) | 9 | (12.3) | 56 | (18.1) |
| Asian | | 0 | (0) | 0 | (0) | 1 | (1.3) | 0 | (0) | 1 | (0.3) |
| Native Hawaiian or | | 0 | (0) | 0 | (0) | 0 | (0) | 0 | (0) | 0 | (0) |
| Other Pacific Islander | | | | | | | | | | | |
| American Indian or | | 0 | (0) | 0 | (0) | 0 | (0) | 0 | (0) | 0 | (0) |
| Alaska Native | | | | | | | | | | | |
| Unknown | | 0 | (0) | 0 | (0) | 0 | (0) | 0 | (0) | 0 | (0) |
| Other | | 0 | (0) | 0 | (0) | 0 | (0) | 0 | (0) | 0 | (0) |
| Ethnicity | n (missing) | 77 | (0) | 81 | (0) | 79 | (0) | 73 | (0) | 310 | (0) |
| Hispanic or Latino | n (%) | 15 | (19.5) | 15 | (18.5) | 15 | (19.0) | 16 | (21.9) | 61 | (19.7) |
| Non-Hispanic or Latino | | 62 | (80.5) | 66 | (81.5) | 64 | (81.0) | 56 | (76.7) | 248 | (80.0) |
| Unknown | | 0 | (0) | 0 | (0) | 0 | (0) | 1 | (1.4) | 1 | (0.3) |
| Study Eye | n (missing) | 77 | (0) | 81 | (0) | 79 | (0) | 73 | (0) | 310 | (0) |
| OD | n (%) | 34 | (44.2) | 37 | (45.7) | 45 | (57.0) | 36 | (49.3) | | |
| OS | | 43 | (55.8) | 44 | (54.3) | 34 | (43.0) | 37 | (50.7) | 158 | (51.0) |
| HADS Depression Scale | n (missing) | 77 | (0) | 81 | (0) | 79 | (0) | 73 | (0) | 310 | (0) |
| | Mean (SD) | 1.8 | (2.1) | 1.6 | (1.9) | 1.6 | (1.6) | 1.9 | (2.1) | 1.7 | (1.9) |
| | Median | 1.0 | | 1.0 | | 1.0 | | 1.0 | | 1.0 | |
| | Min; Mex | 0; 9 | | 0; 10 | | 0; 7 | | 0; 9 | | 0; 10 | |
| HADS Anxiety Scale | n (missing) | 77 | (0) | 81 | (0) | 79 | (0) | 73 | (0) | 310 | (0) |
| | Mean (SD) | 3.1 | (2.4) | 2.6 | (2.4) | 3.1 | (2.6) | 3.4 | (2.6) | 3.0 | (2.5) |
| | Median | 3.0 | | 2.0 | | 3.0 | | 3.0 | | 3.0 | |
| | Min; Max | 0; 9 | | 0; 8 | | 0; 10 | | 0; 10 | | 0; 10 | |
| Chronic Pain | n (missing) | 77 | (0) | 81 | (0) | 79 | (0) | 73 | (0) | 310 | (0) |
| No Pain | | 64 | (83.1) | 71 | (87.7) | 67 | (84.8) | 64 | (87.7) | 266 | (85.8) |
| Mild Pain | | 13 | (16.9) | 10 | (12.3) | 12 | (15.2) | 9 | (12.3) | 44 | (14.2) |
| Moderate Pain | | 0 | (0) | 0 | (0) | 0 | (0) | 0 | (0) | 0 | (0) |
| Severe Pain | | 0 | (0) | 0 | (0) | 0 | (0) | 0 | (0) | 0 | (0) |
| Intraocular Pressure, | n (missing) | 77 | (0) | 81 | (0) | 79 | (0) | 73 | (0) | 310 | (0) |
| Study Eye (mmHg) | | | | | | | | | | | |
| | Mean (SD) | 15.9 | (3.3) | 15.6 | (3.6) | 15.4 | (2.8) | 15.9 | (3.5) | 15.6 | (3.2) |
| | Median | 15.0 | | 16.0 | | 15.0 | | 16.0 | | 15.0 | |
| | Min; Max | 7; 22 | | 9; 22 | | 9; 21 | | 8; 22 | | 7; 22 | |
| Intraocular Pressure, | n (missing) | 77 | (0) | 80 | (1) | 79 | (0) | 73 | (0) | 309 | (1) |
| Non-Study Eye (mmHg) | | | | | | | | | | | |
| | Mean (SD) | 15.4 | (3.2) | 15.6 | (3.8) | 15.6 | (3.1) | 15.6 | (3.3) | 15.6 | (3.4) |

| | | | | | |
|---|---|---|---|---|---|
| Median | 15.0 | 15.0 | 15.0 | 16.0 | 15.0 |
| Min; Max | 8; 22 | 9; 25 | 10; 21 | 8; 24 | 8; 25 |

HADS = Hospital Anxiety and Depression Scale; OD = oculus dexter (right eye); OS = oculus sinister (right eye); SD = standard deviation.
Note:
Percentages are based on the number of observations in each treatment group and overall.

TABLE 20
Subject Achieving Procedural Sedation (Analysis Set: Full Analysis Set)

| | | MELT-300 | Midazolam 3 mg | Ketamine 50 mg | Placebo |
|---|---|---|---|---|---|
| Study Visit | Statistic | (N = 77) | (N = 81) | (N = 79) | (N = 73) |
| Visit 2 (Day 1) | n (missing) | 77 (0) | 81 (0) | 79 (0) | 73 (0) |
| Yes | n (%) | 63 (81.8) | 52 (64.2) | 50 (63.3) | 26 (35.6) |
| No | | 14 (18.2) | 29 (35.8) | 29 (36.7) | 47 (64.4) |
| Versus Placebo | p-value | <.0001 | 0.0004 | 0.0007 | |
| Versus Ketamine | | 0.0096 | 0.9051 | | |

Note:
P-value is from a Pearson chi-square test between pairs of treatments.
Note:
Success for procedural sedation is defined as achieving target sedation level (Ramsay Sedation Scale [RSS] level 2 or 3) at the start of surgery without need for rescue sedation medication, and no need for intraoperative rescue sedation medication to maintain target sedation level.
Note:
The Ramsay Sedation Scale (RSS) at the ‘start of surgery’ is the last RSS prior to the instillation of standardized topical ocular anesthesia drops.

TABLE 21
Mean Intraoperative Analgesia by the Numeric Pain Rating Scale (Carry Forward) (Analysis Set: Full Analysis Set)

| | | MELT-300 | | Midazolam 3 mg | | Ketamine 50 mg | | Placebo | |
|---|---|---|---|---|---|---|---|---|---|
| Timepoint | Statistic | (N = 77) | | (N = 81) | | (N = 79) | | (N = 73) | |
| Insertion of the speculum | n (missing) | 77 | (0) | 81 | (0) | 79 | (0) | 73 | (0) |
| | Mean (SD) | 1.3 | (1.9) | 1.1 | (1.6) | 1.1 | (1.4) | 1.6 | (2.1) |
| | Median | 1.0 | | 0.0 | | 1.0 | | 1.0 | |
| | Min; Max | 0; 9 | | 0; 7 | | 0; 6 | | 0; 9 | |
| Corneal incision | n (missing) | 76 | (1) | 81 | (0) | 79 | (0) | 73 | (0) |
| | Mean (SD) | 2.3 | (2.5) | 1.8 | (1.9) | 1.8 | (1.8) | 2.1 | (2.3) |
| | Median | 1.5 | | 1.0 | | 1.0 | | 1.0 Min; | |
| | Max | 0; 9 | | 0; 7 | | 0; 7 | | 0; 10 | |
| Insertion of the | n (missing) | 76 | (1) | 81 | (0) | 79 | (0) | 73 | (0) |
| phacoemulsion probe | | | | | | | | | |
| | Mean (SD) | 3.2 | (2.6) | 2.9 | (2.1) | 2.5 | (2.2) | 3.0 | (2.3) |
| | Median | 3.0 | | 3.0 | | 2.0 | | 3.0 | |
| | Min; Max | 0; 9 | | 0; 9 | | 0; 7 | | 0; 10 | |
| Insertion of the synthetic | n (missing) | 76 | (1) | 81 | (0) | 79 | (0) | 72 | (1) |
| replacement lens | | | | | | | | | |
| | Mean (SD) | 3.5 | (2.6) | 3.6 | (2.2) | 3.3 | (2.1) | 3.3 | (2.3) |
| | Median | 3.0 | | 4.0 | | 3.0 | | 3.0 | |
| | Min; Max | 0; 9 | | 0; 9 | | 0; 8 | | 0; 10 | |
| Average of Available | N (missing) | 77 | (0) | 81 | (0) | 79 | (0) | 73 | (0) |
| Timepoints | | | | | | | | | |
| | Mean (SD) | 2.56 | (2.07) | 2.37 | (1.58) | 2.16 | (1.55) | 2.48 | (1.95) |
| | Median | 2.00 | | 2.00 | | 1.75 | | 2.25 | |
| | Min; Max | 0.0; 9.0 | | 0.0; 7.0 | | 0.0; 6.0 | | 0.0; 9.0 | |
| | LS Mean | 2.58 | (0.21) | 2.37 | (0.20) | 2.16 | (0.20) | 2.49 | (0.21) |

| | (StdErr) | | | | | | |
|---|---|---|---|---|---|---|---|
| Versus Placebo | LS Mean | 0.09 | (−0.49, .67) | −0.12 | (−0.69, 0.45) | −0.32 | (−0.90, 0.25) |

| | Difference | | | | | |
|---|---|---|---|---|---|---|
| | (95% CI) | | | | | |
| | p-value | 0.7538 | | 0.6817 | | 0.2713 |
| Versus Ketamine | LS Mean | 0.41 | (−0.15, 0.98) | 0.20 | (−0.36, 0.76) | |

| | Difference | | | |
|---|---|---|---|---|
| | (95% CI) | | | |
| | p-value | 0.1521 | | 0.4766 |
| Versus Midazolam | LS Mean | 0.21 | (−0.35, 0.78) | |

| | |
|---|---|
| Difference | |
| (95% CI) | |
| p-value | 0.4612 |

CI = confidence interval; LS Mean = least squares mean; SD = standard deviation; StdErr = standard error.
Note:
LS Mean, Difference, 95% CI, and p-value are from a repeated measures model with treatment, timepoint, and treatment by timepoint as fixed effects. The model assumes an unstructured covariance.
Note:
Only pain scores prior to use of rescue therapy are included in the summary. Observed pain scores that occur after rescue pain medication are replaced with the most-recent pre-rescue pain scores carried forward.

TABLE 22
Mean Intraoperative Analgesia by the Numeric Pain Rating Scale Adjusted
for Site (Carry For-ward) (Analysis Set: Full Analysis Set)

| | | MELT-300 | | Midazolam 3 mg | | Ketamine 50 mg | | Placebo | |
|---|---|---|---|---|---|---|---|---|---|
| Timepoint | Statistic | (N = 77) | | (N = 81) | | (N = 79) | | (N = 73) | |
| Insertion of the speculum | n (missing) | 77 | (0) | 81 | (0) | 79 | (0) | 73 | (0) |
| | Mean (SD) | 1.3 | (1.9) | 1.1 | (1.6) | 1.3. | (1.4) | 1.6 | (2.1) |
| | Median | 1.0 | | 0.0 | | 1.0 | | 1.0 | |
| | Min; Max | 0; 9 | | 0; 7 | | 0; 6 | | 0; 9 | |
| Corneal incision | n (missing) | 76 | (1) | 81 | (0) | 79 | (0) | 73 | (0) |
| | Mean (SD) | 2.3 | (2.5) | 1.8 | (1.9) | 1.8 | (1.8) | 2.1 | (2.3) |
| | Median | 1.5 | | 1.0 | | 1.0 | | 1.0 | |
| | Min; Max | 0; 9 | | 0; 7 | | 0; 7 | | 0; 10 | |
| Insertion of the | n (missing) | 76 | (1) | 81 | (0) | 79 | (0) | 73 | (0) |
| phacoemulsion probe | | | | | | | | | |
| | Mean (SD) | 3.2 | (2.6) | 2.9 | (2.1) | 2.5 | (2.2) | 3.0 | (2.3) |
| | Median | 3.0 | | 3.0 | | 2.0 | | 3.0 | |
| | Min; Max | 0; 9 | | 0; 9 | | 0; 7 | | 0; 10 | |
| Insertion of the synthetic | n (missing) | 76 | (1) | 81 | (0) | 79 | (0) | 72 | (1) |
| replacement lens | | | | | | | | | |
| | Mean (SD) | 3.5 | (2.6) | 3.6 | (2.2) | 3.3 | (2.1) | 3.3 | (2.3) |
| | Median | 3.0 | | 4.0 | | 3.0 | | 3.0 | |
| | Min; Max | 0; 9 | | 0; 9 | | 0; 8 | | 0; 10 | |
| Average of Available | n (missing) | 77 | (0) | 81 | (0) | 79 | (0) | 73 | (0) |
| Timepoints | Mean (SD) | 2.56 | (2.07) | 2.37 | (1.58) | 2.16 | (1.55) | 2.48 | (1.95) |
| | Median | 2.00 | | 2.00 | | 1.75 | | 2.25 | |
| | Min; Max | 0.0; 9.0 | | 0.0; 7.0 | | 0.0; 6.0 | | 0.0; 9.0 | |
| | LS Mean | 2.29 | (0.24) | 2.12 | (0.24) | 1.89 | (0.25) | 2.20 | (0.26 |
| | (StdErr) | | | | | | | | |
| Versus Placebo | LS Mean | 0.10 | (−0.46, 0.66) | −0.08 | (−0.63, 0.47) | −0.30 | (−0.86, 0.25) | | |

| | Difference | | | | | |
|---|---|---|---|---|---|---|
| | (95% CI) | | | | | |
| | p-value | 0.7281 | | 0.7730 | | 0.2795 |
| Versus Ketamine | LS Mean | 0.40 | (−0.14, 0.95) | 0.22 | (−0.31, 0.76) | |

| | Difference | | | |
|---|---|---|---|---|
| | (95% CI) | | | |
| | p-value | 0.1482 | | 0.4148 |
| Versus Midazolam | LS Mean | 0.18 | (−0.36, 0.72) | |

| | |
|---|---|
| Difference | |
| (95% CI) | |
| p-value | 0.5161 |

CI = confidence interval; LS Mean = least squares mean; SD = standard deviation; StdErr = standard error.
Note:
LS Mean, Difference, 95% CI, and p-value are from a repeated measures model with treatment, timepoint, and treatment by timepoint as fixed effects and covariates for study center. The model assumes an unstructured covariance.
Note:
Only pain scores prior to use of rescue therapy are included in the summary. Observed pain scores that occur after rescue pain medication are replaced with the most-recent pre-rescue pain scores carried forward.

TABLE 23
Mean Intraoperative Analgesia by the Numeric Pain Rating Scale (Observed) (Analysis Set: Full Analysis Set)

| | | MELT-300 | | Midazolam 3 mg | | Ketamine 50 mg | | Placebo | |
|---|---|---|---|---|---|---|---|---|---|
| Timepoint | Statistic | (N = 77) | | (N = 81) | | (N = 79) | | (N = 73) | |
| Insertion of the speculum | n (missing) | 77 | (0) | 81 | (0) | 79 | (0) | 73 | (0) |
| | Mean (SD) | 1.3 | (1.9) | 1.1 | (1.6) | 1.1 | (1.4) | 1.6 | (2.1) |
| | Median | 1.0 | | 0.0 | | 1.0 | | 1.0 | |
| | Min; Max | 0; 9 | | 0; 7 | | 0; 6 | | 0; 9 | |
| Corneal incision | n (missing) | 70 | (7) | 79 | (2) | 75 | (4) | 67 | (6) |
| | Mean (SD) | 2.1 | (2.3) | 1.8 | (1.9) | 1.7 | (1.7) | 1.7 | (1.9) |
| | Median | 1.0 | | 1.0 | | 1.0 | | 1.0 | |
| | Min; Max | 0; 9 | | 0; 7 | | 0; 7 | | 0; 10 | |
| Insertion of the | n (missing) | 56 | (21) | 67 | (14) | 66 | (13) | 58 | (15) |
| phacoemulsion probe | | | | | | | | | |
| | Mean (SD) | 2.6 | (2.2) | 2.5 | (1.9) | 2.1 | (2.0) | 2.3 | (1.7) |
| | Median | 2.0 | | 2.0 | | 2.0 | | 2.0 | |
| | Min; Max | 0; 8 | | 0; 9 | | 0; 7 | | 0; 7 | |
| Insertion of the synthetic | n (missing) | 43 | (34) | 53 | (28) | 53 | (26) | 44 | (29) |
| replacement lens | | | | | | | | | |
| | Mean (SD) | 2.3 | (2.0) | 2.9 | (2.1) | 2.5 | (1.8) | 2.3 | (1.8) |
| | Median | 2.0 | | 3.0 | | 2.0 | | 2.0 | |
| | Min; Max | 0; 8 | | 0; 7 | | 0; 8 | | 0; 8 | |
| Average of Available | n (missing) | 77 | (0) | 81 | (0) | 79 | (0) | 73 | (0) |
| Timepoints | | | | | | | | | |
| | Mean (SD) | 2.27 | (1.85) | 2.18 | (1.45) | 2.00 | (1.36) | 2.32 | (1.82) |
| | Median | 2.00 | | 2.00 | | 1.75 | | 2.25 | |
| | Min; Max | 0.0; 9.0 | | 0.0; 7.0 | | 0.0; 6.0 | | 0.0; 9.0 | |
| | LS Mean | 2.59 | (0.22) | 2.46 | (0.21) | 2.22 | (0.21) | 2.48 | (0.22) |
| | (StdErr) | | | | | | | | |
| Versus Placebo | LS Mean | 0.12 | (−0.51, 0.74) | −0.01 | (−0.62, 0.59) | −0.26 | (−0.87, 0.35) | | |

| | Difference | | | | | |
|---|---|---|---|---|---|---|
| | (95% CI) | | | | | |
| | p-value | 0.7138 | | 0.9626 | | 0.4054 |
| Versus Ketamine | LS Mean | 0.37 | (−0.23, 0.98) | 0.24 | (−0.35, 0.83) | |

| | Difference | | | |
|---|---|---|---|---|
| | (95% CI) | | | |
| | p-value | 0.2251 | | 0.4159 |
| Versus Midazolam | LS Mean | 0.13 | (−0.47, 0.73) | |

| | |
|---|---|
| Difference | |
| (95% CI) | 0.6697 |
| p-value | |

CI = confidence interval; LS Mean = least squares mean; SD = standard deviation; StdErr = standard error.
Note:
LS Mean, Difference, 95% CI, and p-value are from a repeated measures model with treatment, timepoint, and treatment by timepoint as fixed effects and covariates for study center. The model assumes an unstructured covariance.
Note:
Only pain scores prior to use of rescue therapy are included in the summary. Observed pain scores that occur after rescue pain medication are replaced with the most-recent pre-rescue pain scores carried forward.

TABLE 24
Subject-Rated Worst Pain During Surgery by the Numeric Pain Rating
Scale (Carry Forward) (Analysis Set: Full Analysis Set)

| | | MELT-300 | | Midazolam 3 mg | | Ketamine 50 mg | | Placebo | |
|---|---|---|---|---|---|---|---|---|---|
| Endpoint | Statistic | (N = 77) | | (N = 81) | | (N = 79) | | (N = 73) | |
| Worst Pain During Surgery | n (missing) | 76 | (1) | 81 | (0) | 79 | (0) | 73 | (0) |
| | Mean (SD) | 3.9 | (2.6) | 4.0 | (2.2) | 3.7 | (2.0) | 4.0 | (2.2) |

| | Median | 4.0 | | 4.0 | | 4.0 | | 4.0 |
|---|---|---|---|---|---|---|---|---|
| | Min; Max | 0; 9 | | 0; 9 | | 0; 8 | | 0; 10 |
| Versus Placebo | Mean | −0.08 | (−0.87, 0.71) | 0.05 | (−0.66, 0.76) | −0.33 | (−1.00, 0.35) | |

| | Difference | | | | | |
|---|---|---|---|---|---|---|
| | (95% CI) | | | | | |
| | p-value | 0.8433 | | 0.8905 | | 0.3374 |
| Versus Ketamine | Mean | 0.25 | (−0.49, 0.99) | 0.38 | (−0.27, 1.03) | |

| | Difference | | | |
|---|---|---|---|---|
| | (95% CI) | | | |
| | p-value | 0.5022 | | 0.2535 |
| Versus Midazolam | Mean | −0.13 | (−0.89, 0.63) | |

| | |
|---|---|
| Difference | |
| (95% CI) | |
| p-value | 0.7383 |

NPRS = numeric pain rating scale.
Note:
P-value is from a Pearson chi-square test between pairs of treatments.
Note:
Success for analgesia response is defined as NPRS < 4 at the available four intraoperative timepoints and a failure otherwise (any intraoperative pain rescue is assumed to be a failure).

TABLE 25
Subjects Achieving Analgesia Response (Observed) (Analysis Set: Full Analysis Set)

| | | MELT-300 | Midazolam 3 mg | Ketamine 50 mg | Placebo |
|---|---|---|---|---|---|
| Study Visit | Statistic | (N = 77) | (N = 81) | (N = 79) | (N = 73) |
| Visit 2 (Day 1) | n (missing) | 77 (0) | 81 (0) | 79 (0) | 73 (0) |
| Yes | n (%) | 33 (42.9) | 33 (40.7) | 39 (49.4) | 33 (45.2) |
| No | | 44 (57.1) | 48 (59.3) | 40 (50.6) | 40 (54.8) |
| Versus Placebo | p-value | 0.7721 | 0.5761 | 0.6077 | |
| Versus Ketamine | | 0.4148 | 0.2728 | | |
| Versus Midazolam | | 0.7875 | | | |

NPRS = numeric pain rating scale.
Note:
P-value is from a Pearson chi-square test between pairs of treatments.
Note:
Success for analgesia response is defined as NPRS < 4 at the available four intraoperative timepoints and a failure otherwise (any intraoperative pain rescue is assumed to be a failure).

TABLE 26
Subjects Completing the Surgery (Analysis Set: Full Analysis Set)

| | | MELT-300 | Midazolam 3 mg | Ketamine 50 mg | Placebo |
|---|---|---|---|---|---|
| Study Visit | Statistic | (N = 77) | (N = 81) | (N = 79) | (N = 73) |
| Visit 2 (Day 1) | n (missing) | 77 (0) | 81 (0) | 79 (0) | 73 (0) |
| Yes | n (%) | 76 (98.7) | 81 (100.0) | 79 (100.0) | 73 (100.0) |
| No | | 1 (1.3) | 0 (0) | 0 (0) | 0 (0) |
| Versus Placebo | p-value | 0.3286 | — | — | |
| Versus Ketamine | 0.3096 | — | | | |
| Versus Midazolam | 0.3035 | | | | |

Note:
P-value is from a Pearson chi-square test between pairs of treatments.

TABLE 27
Subjects Completing the Surgery without Interruption (Analysis Set: Full Analysis Set

| | | MELT-300 | | Midazolam 3 mg | | Ketamine 50 mg | | Placebo | |
|---|---|---|---|---|---|---|---|---|---|
| Study Point | Statistic | (N = 77) | | (N = 81) | | (N = 79) | | (N = 73) | |
| Visit 2 (Day 1) | n (missing) | 77 | (0) | 81 | (0) | 79 | (0) | 73 | (0) |
| Yes | n (%) | 76 | (98.7) | 81 | (100.0) | 74 | (93.7) | 71 | (97.3) |
| No | | 1 | (1.3) | 0 | (0) | 5 | (6.3) | 2 | (2.7) |

| | | | | |
|---|---|---|---|---|
| Versus Placebo | p-value | 0.5286 | 0.1338 | 0.2915 |
| Versus Ketamine | | 0.1024 | 0.0214 | |
| Versus Midazolam | | 0.3035 | | |

Note:
P-value is from a Pearson chi-square test between pairs of treatments.
Note:
Subjects completing the surgery without interruption for intervention (other than rescue medication) due to pain or anxiety are considered successes.

TABLE 28
Subjects Requiring Rescue Sedative Medication (Analysis Set: Full Analysis Set

| | | MELT-300 | | Midazolam 3 mg | | Ketamine 50 mg | | Placebo | |
|---|---|---|---|---|---|---|---|---|---|
| Study Visit | Statistic | (N = 77) | | (N = 81) | | (N = 79) | | (N = 73) | |
| Visit 2 (Day 1) | n (missing) | 77 | (0) | 81 | (0) | 79 | (0) | 73 | (0) |
| Yes | n (%) | 14 | (18.2) | 28 | (34.6) | 29 | (36.7) | 47 | (64.4) |
| Pre-operatively | | 7 | (9.1) | 21 | (25.9) | 24 | (30.4) | 34 | (46.6) |
| Intraoperatively | | 8 | (10.4) | 7 | (8.6) | 5 | (6.3) | 17 | (23.3) |
| No | | 63 | (81.8) | 53 | (65.4) | 50 | (63.3) | 26 | (35.6) |

| | | | | |
|---|---|---|---|---|
| Versus Placebo | p-value | <.0001 | 0.0002 | 0.0007 |
| Versus Ketamine | 0.0096 | 0.7774 | | |
| Versus Midazolam | 0.0198 | | | |

Note:
P-value is from a Pearson chi-square test between pairs of treatments.
Note:
Subjects completing the surgery without interruption for intervention (other than rescue medication) due to pain or anxiety are considered successes.

TABLE 29
Subjects Requiring Rescue Analgesic Medication (Analysis Set: Full Analysis Set

| | | | | Midazolam | | Ketamine | | | |
|---|---|---|---|---|---|---|---|---|---|
| | | MELT-300 | | 3 mg | | 50 mg | | Placebo | |
| Study Visit | Statistic | (N = 77) | | (N = 81) | | (N = 79) | | (N = 73) | |
| Visit 2 (Day 1) | n (missing) | 77 | (0) | 81 | (0) | 79 | (0) | 73 | (0) |
| Yes | n (%) | 37 | (48.1) | 35 | (43.2) | 32 | (40.5) | 33 | (45.2) |
| No | | 40 | (51.9) | 46 | (56.8) | 47 | (59.5) | 40 | (54.8) |

| | | | | |
|---|---|---|---|---|
| Versus Placebo | p-value | 0.7269 | 0.8033 | 0.5585 |
| Versus Ketamine | | 0.3428 | 0.7289 | |
| Versus Midazolam | | 0.5413 | | |

Note:
P-value is from a Pearson chi-square test between pairs of treatments.

TABLE 30
Subjects Wanting Study Drug Again for a Second
Surgery (Analysis Set: Full Analysis Set

| | | | | Midazolam | | Ketamine | | | |
|---|---|---|---|---|---|---|---|---|---|
| | | MELT-300 | | 3 mg | | 50 mg | | Placebo | |
| Endpoint | Statistic | (N = 77) | | (N = 81) | | (N = 79) | | (N = 73) | |
| Want Study | n (missing) | 76 | (1) | 81 | (0) | 77 | (1) | 71 | (1) |
| Drug Again | Mean (SD) | 7.9 | (3.1) | 8.4 | (2.8) | 7.6 | (3.7) | 7.7 | (3.2) |

| | | | | | |
|---|---|---|---|---|---|
| | Median | 10.0 | 10.0 | 10.0 | 9.0 |
| | Min; Max | 0; 10 | 0; 10 | 0; 10 | 0; 10 |
| Versus Placebo | Mean Difference | 0.26 | 0.75 | −0.05 | |
| | (95% CI) | (−0.76, 1.28) | (−0.21, 1.70) | (−1.18, 1.08) | |
| | p-value | 0.6164 | 0.1252 | 0.9284 | |
| Versus Ketamine | Mean Difference | 0.31 | 0.80 | | |
| | (95% CI) | (−0.78, 1.40) | (−0.24, 1.83) | | |
| | p-value | 0.5740 | 0.1311 | | |
| Versus Midazolam | Mean Difference | −0.49 | | | |
| | (95% CI) | (−1.41, 0.43) | | | |
| | p-value | 0.2981 | | | |

CI = confidence interval; SD = standard deviation.
Note:
Scale was measured using 11-point Likert scale of 0 (not likely at all) to 10 (extremely likely).
Note:
Difference, 95% CI, and p-value are from an unpaired t-test with a prior test for a common variance. Reference: Listing 16.2.6.4.

TABLE 31
Subjects Receiving Opioid Concomitant Medication
Postoperatively (Analysis Set: Full Analysis Set

| | | | | Midazolam | | Ketamine | | | |
|---|---|---|---|---|---|---|---|---|---|
| | | MELT-300 | | 3 mg | | 50 mg | | Placebo | |
| Endpoint | Statistic | (N = 77) | | (N = 81) | | (N = 79) | | (N = 73) | |
| Received Postoperative | n | 77 | | 81 | | 79 | | 73 | |
| Opioid on Day 2 | | | | | | | | | |
| Yes | n (%) | 77 | (0.0) | 81 | (0.0) | 79 | (0.0) | 73 | (0.0) |
| No | p-value | (100.0) | | (100.0) | | (100.0) | | (100.0) | |
| Versus Placebo | | — | | — | | — | | | |
| Versus Ketamine | | — | | — | | | | | |
| Versus Midazolam | | — | | | | | | | |
| Total Dose of Opioids | n (missing) | 0 | (0) | 0 | (0) | 0 | (0) | 0 | (0) |
| Taken Postoperatively | | | | | | | | | |
| on Day 2 (mg) | | | | | | | | | |
| | Mean (SD) | | | | | | | | |
| | Median | | | | | | | | |
| | Min; Max | | | | | | | | |
| Versus Placebo | Mean Difference | — | | — | | — | | | |
| | (95% CI) | | | | | | | | |
| | p-value | | | | | | | | |
| Versus Ketamine | Mean Difference | — | | — | | | | | |
| | (95% CI) | | | | | | | | |
| | p-value | | | | | | | | |
| Versus Midazolam | Mean Difference | — | | | | | | | |
| | (95% CI) | | | | | | | | |
| | p-value | | | | | | | | |
| Received Postoperative | n | 77 | | 81 | | 79 | | 73 | |
| Opioid on Day 2-8 (mg) | | | | | | | | | |
| Yes | n (%) | 0 | (0.0) | 0 | (0.0) | 0 | (0.0) | 1 | (1.4) |
| No | | 77 | (100.0) | 81 | (100.0) | 79 | (100.0) | 72 | (98.6) |
| Versus Placebo | p-value | 0.3028 | | 0.2906 | | 0.2966 | | | |
| Versus Ketamine | | — | | — | | | | | |
| Versus Midazolam | | — | | | | | | | |
| Total Dose of Opioids | n (missing) | 0 | (0) | 0 | (0) | 0 | (0) | 1 | (0) |

| Taken Postoperatively | | | |
|---|---|---|---|
| on Day 2-8 (mg) | | | |
| | Mean (SD) | 0.95 | (—) |

| | | | | | |
|---|---|---|---|---|---|
| | Median | | | | 0.95 |
| | Min; Max | | | | 1.0; 1.0 |
| Versus Placebo | Mean Difference | — | — | — | |
| | (95% CI) | | | | |
| | p-value | | | | |
| Versus Ketamine | Mean Difference | — | — | | |
| | (95% CI) | | | | |
| | p-value | | | | |
| Versus Midazolam | Mean Difference | — | | | |
| | (95% CI) | | | | |
| | p-value | | | | |

CI = confidence interval; SD = standard deviation.
Note:
P-value for receipt of postoperative opioid is from a Pearson chi-square test between pairs of treatments.
Note:
Difference, 95% CI, and p-value are from an unpaired t-test with a prior test for a common variance.
Note:
Total dose is the sum of all opioids taken in milligrams for each subject in IV morphine mg equivalent.

TABLE 32
Treatment-Emergent Ocular Adverse Events by
System Organ Class and Preferred Term (Eye:
Study Eye; Analysis Set: Full Analysis Set)

MedDRA v24.1 -

| September 2021 | MELT- | | Midazolam | | Ketamine | | | |
|---|---|---|---|---|---|---|---|---|
| System Organ | 300 | | 3 mg | | 50 mg | | Placebo | |
| Class | (N = 85) | | (N = 87) | | (N = 85) | | (N = 79) | |
| Preferred Term | n (%) | | n (%) | | n (%) | | n (%) | |
| Subjects with Any | 2 | (2.4) | 7 | (8.0) | 5 | (7.1) | 12 | (15.2) |
| Treatment-Emergent | | | | | | | | |
| Ocular AEs | | | | | | | | |
| Eye disorders | 1 | (1.2) | 4 | (4.6) | 5 | (5.9) | 5 | (6.3) |
| Eye pain | 1 | (1.2) | 3 | (3.4) | 3 | (3.5) | 3 | (3.8) |
| Ocular hypertension | 0 | (0) | 1 | (1.1) | 1 | (1.2) | 1 | (1.3) |
| Corneal oedema | 0 | (0) | 0 | (0) | 1 | (1.2) | 1 | (1.3) |
| Foreign body | 0 | (0) | 0 | (0) | 0 | (0) | 1 | (1.3) |
| sensation in eyes | | | | | | | | |
| General disorders | 0 | (0) | 0 | (0) | 0 | (0) | 1 | (1.3) |
| and administration | | | | | | | | |
| site conditions | | | | | | | | |
| Pain | 0 | (0) | 0 | (0) | 0 | (0) | 1 | (1.3) |
| infections and | 0 | (0) | 0 | (0) | 1 | (1.2) | 0 | (0) |
| infestations | | | | | | | | |
| Endophthalmitis | 0 | (0) | 0 | (0) | 1 | (1.2) | 0 | (0) |
| Injury, poisoning | 1 | (1.2) | 1 | (1.1) | 0 | (0) | 3 | (3.8) |
| and procedural | | | | | | | | |
| complications | | | | | | | | |
| Corneal abrasion | 1 | (1.2) | 0 | (0) | 0 | (0) | 0 | (0) |
| Procedural pain | 0 | (0) | 1 | (1.1) | 0 | (0) | 0 | (0) |
| Cataract operation | 0 | (0) | 0 | (0) | 0 | (0) | 3 | (3.8) |
| complication | | | | | | | | |
| Investigations | 0 | (0) | 2 | (2.3) | 1 | (1.2) | 3 | (3.8) |
| Intraocular pressure | 0 | (0) | 2 | (2.3) | 1 | (1.2) | 2 | (2.5) |
| increased | | | | | | | | |

AE = adverse event; MedDRA = Medical Dictionary for Regulatory Activities.
Note:
Treatment-emergent adverse events (TEAE) are defined as events that start on or after the date of study drug placement through the end of Visit 4 or early withdrawal.
Note:
Subjects with one or more adverse events within a level of MedDRA are counted only once in that level.
Note:
Percentages are based on the number of subjects in each treatment.

TABLE 33
Treatment-Emergent Ocular Adverse Events by System
Organ Class and Preferred Term (Eye: Non-Study
Eye; Analysis Set: Full Analysis Set)

| MedDRA v24.1 - | MELT- | | Midazolam | | Ketamine | | | |
|---|---|---|---|---|---|---|---|---|
| September 2021 | 300 | | 3 mg | | 50 mg | | Placebo | |
| System Organ Class | (N = 85) | | (N = 87) | | (N = 85) | | (N = 79) | |
| Preferred Term | n (%) | | n (%) | | n (%) | | n (%) | |
| Paracentesis eye | 0 | (0) | 0 | (0) | 0 | (0) | 1 | (1.3) |
| Subjects with Any | 1 | (1.2) | 1 | (1.1) | 1 | (1.2) | 0 | (0) |
| Treatment-Emergent | | | | | | | | |
| Ocular AEs | | | | | | | | |
| Eye disorders | 1 | (1.2) | 1 | (1.1) | 0 | (0) | 0 | (0) |
| Eye pain | 1 | (1.2) | 0 | (0) | 0 | (0) | 0 | (0) |
| Vision blurred | 0 | (0) | 1 | (1.1) | 0 | (0) | 0 | (0) |
| Infections and | 0 | (0) | 0 | (0) | 1 | (1.2) | 0 | (0) |
| infestations | | | | | | | | |
| Conjunctivitis | 0 | (0) | 0 | (0) | 1 | (1.2) | 0 | (0) |
| bacterial | | | | | | | | |

AE = adverse event; MedDRA = Medical Dictionary for Regulatory Activities.
Note:
Treatment-emergent adverse events (TEAE) are defined as events that start on or after the date of study drug placement through the end of Visit 4 or early withdrawal.
Note:
Subjects with one or more adverse events within a level of MedDRA are counted only once in that level.
Note:
Percentages are based on the number of subjects in each treatment.

TABLE 34
Treatment-Emergent Ocular Adverse Events by
System Organ Class and Preferred Term (Eye:
Either Eye; Analysis Set: Full Analysis Set)

| MedDRA v24.1 - | MELT- | | Midazolam | | Ketamine | | | |
|---|---|---|---|---|---|---|---|---|
| September 2021 | 300 | | 3 mg | | 50 mg | | Placebo | |
| System Organ Class | (N = 85) | | (N = 87) | | (N = 85) | | (N = 79) | |
| Preferred Term | n (%) | | n (%) | | n (%) | | n (%) | |
| Subjects with Any | 3 | (3.5) | 8 | (9.2) | 7 | (8.2) | 12 | (15.2) |
| Treatment-Emergent | | | | | | | | |
| Ocular AEs | | | | | | | | |
| Eye disorders | 2 | (2.4) | 5 | (5.7) | 5 | (5.9) | 5 | (6.3) |
| Eye pain | 2 | (2.4) | 3 | (3.4) | 3 | (3.5) | 3 | (3.8) |
| Ocular hypertension | 0 | (0) | 1 | (1.1) | 1 | (1.2) | 1 | (1.3) |
| Vision blurred | 0 | (0) | 1 | (1.1) | 0 | (0) | 0 | (0) |
| Corneal oedema | 0 | (0) | 0 | (0) | 1 | (1.2) | 1 | (1.3) |
| Foreign body | 0 | (0) | 0 | (0) | 0 | (0) | 1 | (1.3) |
| sensation in eyes | | | | | | | | |
| General disorders | 0 | (0) | 0 | (0) | 0 | (0) | 1 | (1.3) |
| and administration | | | | | | | | |
| site conditions | | | | | | | | |
| Pain | 0 | (0) | 0 | (0) | 0 | (0) | 1 | (1.3) |
| Infections and | 0 | (0) | 0 | (0) | 2 | (2.4) | 0 | (0) |
| infestations | | | | | | | | |
| Conjunctivitis | 0 | (0) | 0 | (0) | 1 | (1.2) | 0 | (0) |
| bacterial | | | | | | | | |
| Endophthalmitis | 0 | (0) | 0 | (0) | 1 | (1.2) | 0 | (0) |
| Injury, poisoning | 1 | (1.2) | 1 | (1.1) | 0 | (0) | 3 | (3.8) |
| and procedural | | | | | | | | |
| complications | | | | | | | | |
| Corneal abrasion | 1 | (1.2) | 0 | (0) | 0 | (0) | 0 | (0) |
| Procedural pain | 0 | (0) | 1 | (1.1) | 0 | (0) | 0 | (0) |
| Cataract operation | 0 | (0) | 0 | (0) | 0 | (0) | 3 | (3.8) |
| complication | | | | | | | | |
| Investigations | 0 | (0) | 2 | (2.3) | 1 | (1.2) | 3 | (3.8) |
| Intraocular pressure | 0 | (0) | 2 | (2.3) | 1 | (1.2) | 2 | (2.5) |
| increased | | | | | | | | |
| Paracentesis eye | 0 | (0) | 0 | (0) | 0 | (0) | 1 | (1.3) |

AE = adverse event; MedDRA = Medical Dictionary for Regulatory Activities.
Note:
Treatment-emergent adverse events (TEAE) are defined as events that start on or after the date of study drug placement through the end of Visit 4 or early withdrawal.
Note:
Subjects with one or more adverse events within a level of MedDRA are counted only once in that level;
Note:
Percentages are based on the number of subjects in each treatment.

TABLE 35
Treatment-Emergent Ocular Adverse Events of Special
Interest by System Organ Class and Preferred
Term (Analysis Set: Full Analysis Set)

| | | | | |
|---|---|---|---|---|
| MedDRA v24.1 - | MELT- | Midazolam | Ketamine | Placebo |
| September 2021 | 300 | 3 mg | 50 mg | (N = 79) |
| System Organ Class | (N = 85) | (N = 87) | (N = 85) | n (%) |
| Preferred Term | n (%) | n (%) | n (%) | |

No subjects meet the criteria for this table.
AE = adverse event; MedDRA = Medical Dictionary for Regulatory Activities.
Note:
Treatment-emergent adverse events (TEAE) are defined as events that start on or after the date of study drug placement through the end of Visit 4 or early withdrawal.
Note:
Subjects with one or more adverse events within a level of MedDRA are counted only once in that level.
Note:
Percentages are based on the number of subjects in each treatment.

### Conclusion:

MELT-300 (e.g., 3 mg midazolam and 50 mg ketamine) sublingual tablet achieved topline results and primary sedation endpoint in the Phase 2 efficacy and safety study. MELT-300 was statistically superior for procedural sedation compared to all comparator treatment arms, including midazolam 3 mg (p=0.0129) and ketamine 50 mg (p=0.0096).

MELT-300 treatment arm was 66% less likely to require rescue sedation pre-operatively compared to the midazolam treatment arm. The MELT-300 treatment arm was also 50% less likely to require rescue sedation compared to midazolam (p=0.0198). Moreover, MELT-300 treatment arm had zero serious adverse events (N=77).

From this study, MELT-300 was shown to be a promising non-IV option for procedural sedation (e.g., for cataract procedures and other potential uses).

### Example 3: MELT-300 Sublingual Tablet Phase III Efficacy and Safety Study

### Introduction

A Phase 3 efficacy and safety study of the MELT-300 formulation for procedural sedation in subjects undergoing cataract extraction with lens replacement (CELR) was conducted at twelve sites with approximately 528 enrolled subjects. MELT-300 is a sublingual, needle- and opioid-free formulation for procedural sedation during cataract surgery, which combines fixed doses of midazolam (3 mg) and ketamine (50 mg) in one rapidly dissolving tablet (RDT) that is administered sublingually for procedural sedation. MELT-300 utilizes fast-dissolving delivery technology (Zydis®, Catalent Inc.) to rapidly dissolve the tablet for absorption across the sublingual mucosa.

### Detailed Summary

An aim of the clinical trial was to determine MELT-300 efficacy for procedural sedation in adult participants undergoing CELR. The clinical trial investigates the safety of MELT-300. MELT-300 was compared to a similar placebo, and a comparator (sublingual midazolam) to confirm the benefit of inclusion of ketamine in the combined drug product.

Additional aims of the clinical trial include determining: 1) effectiveness of MELT-300 in comparison to placebo on procedural sedation for cataract surgery; 2) effectiveness of MELT-300 compared with midazolam on procedural sedation, to determine the contribution of ketamine component and inform the risk of ketamine in MELT-300; 3) time to achieve preoperative target sedation level with MELT-300; and 4) which medical problems participants have when taking MELT-300 vs placebo.

Eligible participants were admitted to the study unit on Day 1. Participants were randomized prior to surgery 4:1:1 to: 1) MELT-300 (i.e., a single MELT-300 sublingual tablet which contains 3 mg midazolam and 50 mg of ketamine); 2) Midazolam (i.e., a single matching midazolam sublingual tablet, which contains 3 mg midazolam); and 3) Placebo (i.e., a single matching placebo sublingual tablet) The study was a Phase 3, randomized, double-masked, placebo-controlled, parallel-cohort, multicenter study.

Participants received study medication 30 (+5) minutes, without food or water, before planned surgery start (defined as instillation of topical ocular anesthetic gel (3 drops of chloroprocaine hydrochloride ophthalmic gel)).

The effectiveness of MELT-300 was evaluated after study medication is administered before surgery, in the course of surgery (intraoperatively), and postoperative on Day 1 (end of surgery defined as just prior to drape removal). Efficacy assessments included assessments of sedation, need for rescue medication for sedation, need for rescue medication for pain, and the ability to complete the surgery.

The safety of MELT-300 was performed at baseline, in the course of surgery (intraoperatively), postoperatively on Day 1, and on Day 3+1 day post dose of study medication. Safety assessment included monitoring of AEs, vital sign measurements, and physical examinations.

Comparisons were made of MELT-300 administration against: (i) placebo alone; (ii) sublingually delivered midazolam alone; and (iii) sublingually delivered ketamine alone. Primary efficacy endpoints included appropriate sedation during the cataract surgery and management of intraoperative pain during the surgery.

### Eligibility Criteria

Participants met at least the following criteria in order to be enrolled into the study:
1) Males and females≥18 years of age.
2) Were to undergo unilateral primary CELR under topical anesthesia, with a phacoemulsification device and insertion of an intraocular lens (no restrictions on lens type).
3) For women of childbearing potential (WOCBP), had a negative urine pregnancy test, and abstain from sexual activity or use a double barrier method (e.g. condom and diaphragm) of birth control from Day 1 and up to 2 days after study drug administration.
4) Were willing to refrain from alcohol consumption within 24 hours of randomization.
5) Were competent to provide informed consent.
6) Voluntarily provided informed consent in accordance with governing International Review Board (IRB) requirements and provide Health Insurance Portability and Accountability Act (HIPAA) authorization, prior to any procedures or evaluations performed specifically for the sole purpose of the study.
7) Indicate they understand and are able, willing, and likely to fully comply with study procedures and restrictions.

Participants were excluded from the clinical trial if they met at least any of the following criteria:
1) Subjects scheduled for simultaneous bilateral or 2nd-eye cataract surgery (subjects scheduled for a future 2nd eye cataract surgery are eligible for the study).
2) Known sensitivity to benzodiazepines or ketamine.
3) Known sensitivity to-caines (including proparacaine, ester-type local anesthetics), benzalkonium chloride (BAK).
4) Intraocular pressure (IOP)>30 mmHg in the study eye or fellow eye at screening.
5) History of iritis, or any ocular trauma with iris damage in the study eye.
6) Presence of active corneal pathology other than dry eye per slit lamp and external eye exam at screening in either eye.
7) Presence of extraocular/intraocular inflammation in either eye.
8) Presence of active bacterial and/or viral infection in either eye.
9) History of intraocular non-laser surgery in the study eye within the 3 months prior to day of surgery, or intraocular laser surgery in the study eye within 30 days prior to the day of surgery.
10) Requiring or planning other additional ocular surgery during the cataract surgery (e.g. glaucoma surgery ([minimally invasive or traditional], limbal relaxing incisions, etc.) or performing laser-assisted CELR.
11) Presence of active infection, mucositis, cold sores, canker sores, vesicles, viral lesions, local irritation/inflammation, or periodontal disease of the oral cavity. In addition, evidence of piercings of the tongue or anywhere in the oral cavity, his-tory of oral cavity piercings, history of significant dental disease, or history of dysphagia.
12) Women who are nursing a child or plan to nurse a child during the study.
13) Have a history or clinical manifestations (e.g., signs, symptoms, laboratory values, diagnostic imaging, etc.) of significant gastrointestinal, cardiovascular, hepatic, renal, hematological, endocrine, neurological, psychiatric, respiratory, or other medical condition that in the opinion of the investigator might confound the study results or pose additional risk in administering the study procedures.
14) Use of disallowed medications including the following:
   a. Antihypertensive agent or diabetic regimen at a dose that has not been stable for at least 30 days prior to Day 1, or which is not expected to remain stable throughout the study.
   b. Central nervous system (CNS) active drugs such as benzodiazepines, tricyclic antidepressants, serotonin and norepinephrine reuptake inhibitors (SNRIs), or selective serotonin reuptake inhibitors (SSRIs) that have not been stable for at least 30 days prior to Day 1, or which is not expected to remain stable throughout the study.
   c. Initiating the use of, switching to a different, or increasing the dose of a sleep medication (e.g. lorazepam, zolpidem, etc.) within 3 days of randomization.
15) Illicit drug use or alcohol abuse based on medical history, or currently engaged in il-licit drug use or alcohol abuse.
   a. Alcohol abuse is defined as 5 or more drinks in one sitting or 15 or more drinks in a week for men and 4 or more drinks in one sitting or 8 or more drinks in a week for women. A drink is considered a 1.5 oz shot, 12 oz of beer, or 5 oz of wine.
   b. However, patients with a medical history of illicit drug use or alcohol abuse≥5 years prior to the time of screening and who have recovered and have been drug/alcohol free for at least that period of time (i.e., 5 years) can be enrolled C. Patients with a medical history of medical or recreational marijuana (including THC and/or CBD) use≥1 year prior to the time of screening and have been marijuana free for at least that period of time (i.e. 1 year) can be enrolled.
16) Creatinine clearance rate<60 mL/min estimated using the CKD-EPI 2021cr (NKD) equation
17) Aspartate aminotransferase (AST), alanine aminotransferase (ALT), or alkaline phosphatase (ALP)>2.5 times upper limit of normal (ULN), or total bilirubin>1.5×ULN. In cases of documented Gilbert syndrome, subjects with elevated bilirubin levels will be permitted to enroll in the study if other liver function tests are with-in the specified limits.
18) Any other abnormal laboratory results or presence of any condition that the Investigator believes would put the subject at risk or confound the interpretation of results.

Detailed descriptions of the study design arms and interventions, as well as primary and secondary outcome measures are provided in TABLES 36-38.

TABLE 36
Phase III Arms and Intervention

| Participant Group/Arm | Intervention/Treatment |
|---|---|
| Experimental: MELT-300 | Drug: MELT-300 sublingual tablet |
| sublingual tablet | Each dose of MELT-300 provided as |
| Participants received a | a single sublingual tablet, |
| single dose of MELT-300 | containing 3 mg midazolam and |
| sublingual, tablet | 50 mg ketamine. Participants |
| containing 3 mg of | received 1 sublingual tablet of |
| midazolam and 50 mg | study medication 30 (±5) minutes |
| of ketamine. | prior to planned surgery start, |
| | without food or water. |
| Active Comparator: | Drug: Midazolam sublingual tablet |
| Midazolam sublingual | Each dose of midazolam provided as |
| tablet | a single sublingual tablet, |
| Participants received a | containing 3 mg midazolam. |
| single dose of midazolam | Participants will receive 1 sublingual |
| 3 mg sublingual tablet. | tablet of study medication 30 (±5) |
| Placebo Comparator: | minutes prior to planned surgery |
| Placebo sublingual tablet | start, without food or water. |
| Participants received a | Drug: Placebo sublingual tablet |
| single dose of a matching | Each dose of placebo provided as a |
| placebo sublingual tablet. | matching sublingual tablet, |
| | containing placebo. |
| | Participants will receive 1 sublingual |
| | tablet of study medication 30 (±5) |
| | minutes prior to planned surgery |
| | start, without food or water. |

TABLE 37
Phase III Primary Outcome Measures

| Outcome | | |
|---|---|---|
| Measure | Measure Description | Time Frame |
| Percentage of | Successful procedural sedation was | Preoperative |
| Participants | defined as achieving target sedation | (Day 1), |
| Achieving | level (Ramsay Sedation Scale | Intraoperative |
| Successful | [RSS] level 2 or 3) by the start of | (Day 1), and |
| Procedural | surgery without need for rescue | Postoperative |
| Sedation | sedation medication, no | (Day 1) |
| | requirement for intraoperative | |
| | sedation medication, and able to | |
| | complete the surgery (i.e. | |
| | procedural sedation responder). | |
| | The Ramsay Sedation Scale (RSS) | |
| | was the first scale to be defined for | |
| | the sedated participants and was | |
| | designed as a test of arousability. | |
| | The Ramsay Scale provides three | |
| | levels of ‘awake’ states (score 1-3) | |
| | and three levels of ‘asleep’ states | |
| | (score 4-6). A score of 2 | |
| | (participant is cooperative, | |
| | orientated, and tranquil) best fits an | |
| | optimum sedation level based on | |
| | the criteria of calm, comfortable, | |
| | communicative, and cooperative | |
| | participants. | |

TABLE 38
Phase III Secondary Outcome Measures

| Outcome Measure | Measure Description | Time Frame |
|---|---|---|
| Percentage of Participants | Rescue sedation medication (i.e., | Preoperative (Day 1) and |
| Requiring Rescue Sedation | intravenous midazolam) at a dose | Intraoperative (Day 1) |
| Medication | determined by Investigator or | |
| | anesthesiologist was given, if needed. | |
| | Use of rescue sedation medication | |
| | during surgery was allowed if a | |
| | participant's RSS score is <2. | |
| Percentage of Participants | Participants achieving target sedation | Preoperative (Day 1) |
| Achieving Preoperative | level RSS level 2 or 3 by the start of | |
| Procedural Sedation | surgery without need for rescue | |
| Without Need For Rescue | sedation medication. | |
| Sedation Medication | | |
| Percentage of Participants | Rescue sedation medication (i.e., | Preoperative (Day 1) |
| Requiring Rescue Sedation | intravenous midazolam) at a dose | |
| Medication Preoperatively | determined by Investigator or | |
| | anesthesiologist was given, if needed. | |
| | Use of rescue sedation medication | |
| | during surgery was allowed if a | |
| | participant's RSS score is <2. | |
| Percentage of Participants | Rescue sedation medication (i.e., | Intraoperative (Day 1) |
| Requiring Rescue Sedation | intravenous midazolam) at a dose | |
| Medication Intraoperatively | determined by Investigator or | |
| | anesthesiologist was given, if needed. | |
| | Use of rescue sedation medication | |
| | during surgery was allowed if a | |
| | participant's RSS score is <2. | |
| Assessment of Sedation | Target sedation level (RSS level 2 or | Preoperative (Day 1), |
| Scores in Participants | 3) by the start of surgery without | Intraoperative (Day 1), and |
| Without Requiring Rescue | need for rescue sedation medication, | Postoperative (Day 1) |
| Sedation Medication | no requirement for intraoperative | |
| | sedation medication, and able to | |
| | complete the surgery (i.e. procedural | |
| | sedation responder). | |
| Percentage of Participants | Participants achieving the target | Postoperative (Day 1) |
| Able to Complete the | sedation level (RSS level 2 or 3) by | |
| Surgery | the start of surgery without need for | |
| | rescue sedation medication, no | |
| | requirement for intraoperative | |
| | sedation medication. | |
| Percentage of Participants | | Postoperative (Day 1) |
| Able to Complete the | | |
| Surgery Without | | |
| Intervention (Other Than | | |
| Rescue Sedation | | |
| Medication) | | |
| Duration to Achieve | Measurement of duration (time | Preoperative (Day 1) |
| Preoperative Target | taken) to achieve target sedation | |
| Sedation (RSS level 2 or 3) | level (RSS level 2 or 3) by the | |
| | start of surgery. | |
| Percentage of Participants | An adverse event was any untoward | Baseline, Preoperative (Day 1), |
| Reporting Treatment | medical occurrence in a participant | Intraoperative (Day 1), |
| Emergent Adverse Events | or clinical study participant, | Postoperative (Day 1), and |
| (TEAEs) | temporally associated with the use of | Day 3 ± 1 post dose of |
| | study intervention, whether or not | study medication |
| | considered related to the study | |
| | intervention. | |
| Percentage of Participants | The prespecified AESI included: | Baseline, Preoperative (Day 1), |
| Reporting Adverse Events | 1. AEs representing ketamine | Intraoperative (Day 1), |
| Special Interest (AESI) | emergence delirium (i.e., | Postoperative (Day 1), and |
| | hallucinations, unpleasant/bad | Day 3 ± 1 post dose of |
| | dreams, vivid imagery, | study medication |
| | confusional state, excitement, | |
| | behavior abnormal, agitation). | |
| | 2. Respiratory AEs (i.e., respiratory | |
| | depression, hypoventilation, dyspnea, | |
| | oxygen desaturation, respiratory | |
| | arrest, airway obstruction). | |
| | 3. Cardiovascular AEs (i.e., | |
| | hypotension, blood pressure | |
| | increased, hypertension [new onset | |
| | or worsened], bradycardia/tachycardia, | |
| | cardiac arrest, cardiac | |
| | decompensation). | |
| | 4. Neurocognitive AEs (i.e., | |
| | confusional state, amnesia, memory | |
| | impairment, attention impaired, | |
| | disorientation, delirium). | |
| | 5. Abuse-related AEs. | |
| | 6. Oversedation (i.e., sedation | |
| | excessive) requiring the use of | |
| | flumazenil | |
| | Oral/pharyngeal AEs (i.e.. changes to | |
| | tongue, oral cavity [active infection, | |
| | mucositis, cold sores, canker sores, | |
| | vesicles, viral lesions, stomatitis, | |
| | periodontal disease], and/or | |
| | oral/pharyngeal function | |
| | [hypoesthesia, taste disorders, | |
| | dysphagia]). | |
| Mean Change from Baseline | | Baseline, Preoperative |
| in Blood Pressure (mmHg) | | (Day 1), Intraoperative |
| | | (Day 1), Postoperative |
| | | (Day 1), and Day 3 ± 1 |
| | | post dose of study medication |
| Mean Change from Baseline | | Baseline, Preoperative (Day 1), |
| in Heart Rate [beats per | | Intraoperative (Day 1), |
| minute (bpm)] | | Postoperative (Day 1), and |
| | | Day 3 ± 1 post dose of |
| | | study medication |
| Mean Change from Baseline | | Baseline, Preoperative (Day 1), |
| in Respiratory Rate (breath | | Intraoperative (Day 1), |
| per minute) | | Postoperative (Day 1), and |
| | | Day 3 ± 1 post dose of |
| | | study medication |
| Mean Change from Baseline | | Baseline, Preoperative (Day 1), |
| in Body Temperature | | Intraoperative (Day 1), |
| (degrees Fahrenheit) | | Postoperative (Day 1), and |
| | | Day 3 ± 1 post dose of |
| | | study medication |
| Mean Change from Baseline | | Baseline, Preoperative (Day 1), |
| in Pulse Oximetry (%) | | Intraoperative (Day 1), |
| | | Postoperative (Day 1), and |
| | | Day 3 ± 1 post dose of |
| | | study medication |

### Results:

A summary of the disposition of all subjects in the analysis set is provided in TABLE 39. Overall, 828 subjects were screened, with 531 subjects passing the screen (i.e., were randomized) and 297 subjects failing the screens for various exclusion reasons listed in TABLE 39, referencing the exclusion type numbers from TABLE 38. Total subjects included in the intent-to-treat (ITT) analysis set (e.g., all randomized subjects) was N=531, with treatment arms of MELT-300 (N=353), midazolam (N=89), and placebo (N=89). Total subjects included in the safety analysis set (e.g., all randomized subjects who received any study drug) was N=527, with treatment arms of MELT-300 (N=350), midazolam (N=89), and placebo (N=88). Total subjects included in the all-treated analysis set (e.g., all randomized subjects who received any study drug) was N=527, with treatment arms of MELT-300 (N=350), midazolam (N=89), and placebo (N=88). Total subjects included in the per-protocol (PP) analysis set (e.g., all randomized subjects who received any study drug and who had no significant protocol deviations that could complicate interpretation of efficacy and/or safety) was N=523, with treatment arms of MELT-300 (N=347), midazolam (N=89), and placebo (N=87).

A summary of protocol deviations in the intention-to-treat (ITT) dataset are provided in TABLE 40. Percentages shown for site-level protocol deviations were based on the number of sites.

Demographics and subject baseline characteristics of the intention-to-treat (ITT) analysis set are provided in TABLE 41, which includes information on subject age, sex, ethnicity, race, caffeine consumption, and history of smoking and drug use.

Achievement of the primary endpoint (i.e., procedural sedation) was evaluated. Success for a procedural sedation responder was defined as a subject that achieved the target sedation level (RSS level 2 or 3) by the start of surgery without the need for rescue sedation medication, no requirement for intraoperative sedation medication, and who was able to complete the surgery. Primary endpoint data for the intention-to-treat (ITT) analysis set is shown in TABLE 42), where comparisons between MELT-300 and midazolam are displayed in the midazolam column, and comparisons between MELT-300 and placebo are displayed in the placebo column. 277/353 (78.5%) subjects receiving MELT-300 achieved target sedation, compared to 58/89 (65.2%) subjects receiving 3 mg midazolam alone, and 32/89 (36.0%) of subjects receiving placebo.

Treatment differences were tested using a Pearson's chi-square test where a p-value<0.05 indicated a difference, with a Fisher's exact test being used instead if any expected values were <5. Treatment difference p-values between the MELT-300 treatment arm and the midazolam or placebo treatment arms were p=0.009 and p<0.001, respectively. Risk difference effects were estimated by the risk difference in proportions (MELT-300-Midazolam, and MELT-300-Placebo) and 95% CIs, calculated using the Miettinen and Nurminen method. A supportive stratified analysis was performed using the Cochran-Mantel-Haenszel (CMH) row-mean score test to stratify by site grouping. A common risk difference effect was estimated by the common risk difference in proportions (MELT-300-Midazolam, and MELT-300-Placebo) and 95% CIs, calculated using the Miettinen and Nurminen method stratified by site grouping.

A sensitivity analysis of the primary endpoint of procedural sedation responders (ITT analysis set) did not reveal any significant differences (not shown). This sensitivity analysis utilized the last observation (collected after study drug administration) carried forward method for imputing missing preoperative RSS scores just prior to the start of surgery.

Analysis of the primary endpoint (e.g., procedural sedation response) was performed on the all-treated and per-protocol (PP) analysis sets, as shown in TABLE 43 and TABLE 44, respectively. In the all-treated analysis set, treatment difference p-values between the MELT-300 treatment arm and the midazolam or placebo treatment arms were p=0.005 and p<0.001, respectively (see TABLE 43). In the per-protocol analysis set, treatment difference p-values between the MELT-300 treatment arm and the midazolam or placebo treatment arms were p=0.004 and p<0.001, respectively (see TABLE 44).

Analysis of the first key secondary analysis of requirement for rescue sedation medication was performed. Requirement for rescue sedation medication was defined as a subject that received rescue sedation medication during the preoperative or intraoperative period. Group comparisons and statistics were analogous to those for TABLES 42-44. Data from the intention-to-treat analysis set is shown in TABLE 45. In the ITT analysis set, 68/353 (19.3%) of subjects receiving MELT-300; 30/89 (33.7%) of subjects receiving midazolam alone; and 55/89 (61.8%) of subjects receiving placebo required rescue sedation medication. Treatment difference p-values between the MELT-300 treatment arm and the midazolam or placebo treatment arms in the ITT analysis set were p=0.003 and p<0.001, respectively. Sensitivity analysis of the ITT analysis set was not conducted due to no subjects reporting the intercurrent event which would trigger the multiple imputation analysis. Data for the requirement for rescue sedation medication in the all-treated analysis set is shown in TABLE 46. In the all-treated analysis set, 68/350 (19.4%) of subjects receiving MELT-300; 30/89 (33.7%) of subjects receiving midazolam alone; and 55/88 (62.5%) of subjects receiving placebo required rescue sedation medication. Treatment difference p-values between the MELT-300 treatment arm and the midazolam or placebo treatment arms in the all-treated analysis set were p=0.004 and p<0.001, respectively. Data for the requirement for rescue sedation medication in the per-protocol (PP) analysis set is shown in TABLE 47. In the PP analysis set, 68/347 (19.6%) of subjects receiving MELT-300; 30/89 (33.7%) of subjects receiving midazolam alone; and 55/87 (63.2%) of subjects receiving placebo required rescue sedation medication. Treatment difference p-values between the MELT-300 treatment arm and the midazolam or placebo treatment arms in the PP analysis set were p=0.004 and p<0.001, respectively.

Analysis of the second key secondary analysis of preoperative procedural sedation responders was performed. A preoperative procedural sedation responder was defined as a subject that achieved the target sedation level (RSS level 2 or 3) by the start of surgery without the need for preoperative rescue sedation medication. Group comparisons and statistics were analogous to those for TABLES 42-47. Data from the intention-to-treat analysis set is shown in TABLE 48. In the ITT analysis set, 289/353 (81.9%) of subjects receiving MELT-300; 65/89 (73.0%) of subjects receiving midazolam alone; and 34/89 (38.2%) of subjects receiving placebo were preoperative procedural sedation responders. Treatment difference p-values between the MELT-300 treatment arm and the midazolam or placebo treatment arms in the ITT analysis set were p=0.062 and p<0.001, respectively. A sensitivity analysis of the primary endpoint of procedural sedation responders (ITT analysis set) did not reveal any significant differences (not shown). Data for preoperative procedural sedation responders in the all-treated analysis set is shown in TABLE 49. In the all-treated analysis set. 289/350 (82.6%) of subjects receiving MELT-300; 65/89 (73.0%) of subjects receiving midazolam alone; and 34/88 (38.6%) of subjects receiving placebo were preoperative procedural sedation responders. Treatment difference p-values between the MELT-300 treatment arm and the midazolam or placebo treatment arms in the all-treated analysis set were p=0.042 and p<0.001, respectively. Data for preoperative procedural sedation responders in the per-protocol (PP) analysis set is shown in TABLE 50. In the PP analysis set, 287/347 (82.7%) of subjects receiving MELT-300; 65/89 (73.0%) of subjects receiving midazolam alone; and 34/87 (39.1%) of subjects receiving placebo were preoperative procedural sedation responders. Treatment difference p-values between the MELT-300 treatment arm and the midazolam or placebo treatment arms in the PP analysis set were p=0.039 and p<0.001, respectively.

A secondary analysis of rescue sedation medication use (both preoperatively and intraoperatively) for the ITT analysis set is shown in TABLE 51. In the ITT analysis set, 37/353 (10.5%) of subjects receiving MELT-300; 16/89 (18.0%) of subjects receiving midazolam alone; and 28/89 (31.5%) of subjects receiving placebo received preoperative rescue sedation medication. Treatment difference p-values between the MELT 300 treatment arm and the midazolam or placebo treatment arms in the ITT analysis set were p=0.052 and p<0.001, respectively. In the ITT analysis set, 30/353 (8.5%) of subjects receiving MELT-300; 15/89 (16.9%) of subjects receiving midazolam alone; and 28/89 (31.5%) of subjects receiving placebo received intraoperative rescue sedation medication. Treatment difference p-values between the MELT-300 treatment arm and the midazolam or placebo treatment arms in the ITT analysis set were p=0.020 and p<0.001, respectively.

A secondary analysis of Ramsay sedation scale (RSS) scores for the ITT analysis set is shown in TABLE 52. The RSS scores are summarized for: the baseline, just prior to the start of surgery, the minimum and maximum preoperative score, the minimum and maximum intraoperative score, the minimum and maximum postoperative score, and the maximum overall score.

Evaluation of time to achieve preoperative target sedation level for the all-treated analysis set is shown in TABLE 53. The data provided includes the time to achieve preoperative target sedation level, which was defined as the time from the administration of study drug until the first Ramsay Sedation Scale (RSS) score of 2 or 3, calculated in minutes. Calculations were for subjects dosed with MELT-300 who achieved a preoperative RSS score of 2 or 3 using the Kaplan-Meier (KM) method. The analysis excluded subjects who receive preoperative rescue sedation medication. The data provided also includes a sensitivity analysis of time to achieve preoperative target sedation level, which was defined as the time from the administration of study drug until the first Ramsay Sedation Scale (RSS) score of 2 or 3, calculated in minutes. Calculations were for subjects dosed with MELT-300 who achieved a preoperative RSS score of 2 or 3 using the KM method. Sensitivity analysis censored subjects who receive preoperative rescue sedation medication at the time rescue sedation medication was first administered.

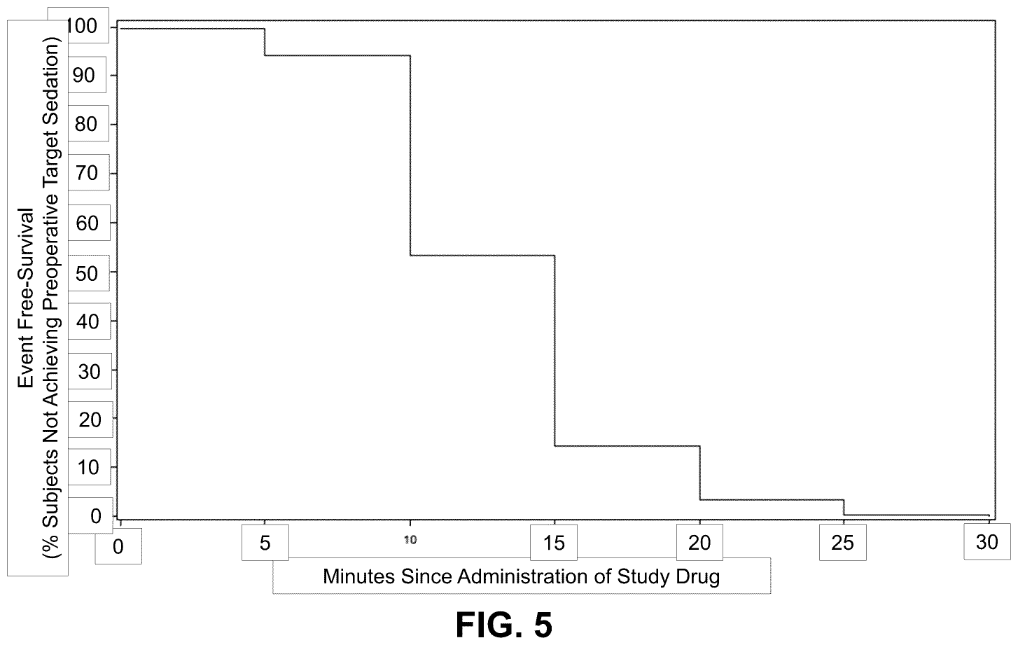

A plot of the Kaplan-Meier Curve for time to achieve preoperative target sedation level for the all-treated analysis set is provided in FIG. 5. A secondary analysis of surgery completion in the intention-to-treat (ITT) analysis set is provided in TABLE 54. In the ITT analysis set, 349/353 (98.9%) of subjects receiving MELT-300; 89/89 (100.0%) of subjects receiving midazolam alone; and 87/89 (97.8%) of subjects completed the surgery, and all of those subjects completing the surgery completed the surgery without intervention due to pain or anxiety.

In the intention-to-treat analysis set, 3/353 (0.8%) of subjects receiving MELT-300; 1/89 (1.1%) of subjects receiving midazolam alone; and 2/89 (2.2%) of subjects received intraoperative supplemental analgesia (data not shown).

Surgeon satisfaction with the operative experience was measured postoperatively on Day 1 using a 5-point Likert scale (5=very satisfied; 4=satisfied; 3=neither satisfied nor dissatisfied; 2=dissatisfied; 1=very dissatisfied). Results are provided in TABLE 55. Comparisons between MELT-300 and midazolam are displayed in the midazolam column, and comparisons between MELT-300 and placebo are displayed in the placebo column. The means were compared using 2-sample t-tests and the Satterthwaite method. The proportion of satisfied responses (‘Very Satisfied’ or ‘Satisfied’, pooled) were tested using a Pearson's chi-square test; and a Fisher's exact test was used instead if any expected values were <5.

Patient satisfaction with the operative experience was measured at the Follow-Up visit using a 5-point Likert scale (5=very satisfied; 4=satisfied; 3=neither satisfied nor dissatisfied; 2=dissatisfied; 1=very dissatisfied). Results are provided in TABLE 56. Comparisons between MELT-300 and midazolam are displayed in the midazolam column, and comparisons between MELT-300 and placebo are displayed in the placebo column. The means were compared using 2-sample t-tests and the Satterthwaite method. The proportion of satisfied responses (‘Very Satisfied’ or ‘Satisfied’, pooled) were tested using a Pearson's chi-square test; and a Fisher's exact test was used instead if any expected values were <5.

An overview of adverse events (AEs) is provided in TABLE 57 for the safety analysis set. Reported data includes an overall summary of the presence of any AEs, treatment-emergent ocular adverse events (TEAEs), treatment-related TEAEs, TEAE of special interest (TEAESI), serious AEs (SEAs), and treatment-emergent SAEs (TESAE). Categories were not mutually exclusive. Results include the number of subjects, percent of subjects, and number of events in each category, respectively. A summary of treatment-emergence adverse events (TEAEs) by location, system organ class, and preferred term for the safety analysis set is provided in TABLE 58. A summary of treatment-related treatment-emergent adverse events (TRTEAEs) by system organ class, and preferred term for the safety analysis set is provided in TABLE 59. No SAEs (overall), TESAEs, or treatment-related TESAEs were observed. No SAEs resulting in death or discontinuation of the study was observed.

TABLE 39
Summary of Disposition (All Subjects)

| | MELT-300 | | Midazolam | | Placebo | | Overall | |
|---|---|---|---|---|---|---|---|---|
| Subjects Screened | | | | | | | 828 | (100.0) |
| Subjects Randomized | | | | | | | 531 | (64.1) |
| Screen Failures | | | | | | | 297 | (35.9) |
| Exclusion 03 | | | | | | | 1 | (0.1) |
| Exclusion 06 | | | | | | | 3 | (0.4) |
| Exclusion 07 | | | | | | | 2 | (0.2) |
| Exclusion 10 | | | | | | | 1 | (0.1) |
| Exclusion 12 | | | | | | | 5 | (0.6) |
| Exclusion 13 | | | | | | | 7 | (0.8) |
| Exclusion 14 | | | | | | | 10 | (1.2) |
| Exclusion 15 | | | | | | | 25 | (3.0) |
| Exclusion 16 | | | | | | | 96 | (11.6) |
| Exclusion 17 | | | | | | | 6 | (0.7) |
| Exclusion 18 | | | | | | | 10 | (1.2) |
| Exclusion 02 | | | | | | | 13 | (1.6) |
| Exclusion 04 | | | | | | | 4 | (0.5) |
| Exclusion 07 | | | | | | | 124 | (15.0) |
| Analysis Sets | | | | | | | | |
| Intent-to-treat (ITT) Analysis Set | 353 | (100.0) | 89 | (100.0) | 89 | (100.0) | 531 | (100.) |
| Safety Analysis | 350 | (99.2) | 89 | (100.0) | 88 | (98.9) | 527 | (99.2) |
| All-Treated Analysis Set | 350 | (99.2) | 89 | (100.0) | 88 | (98.9) | 527 | (99.2) |
| Per-protocol (PP) Analysis Set | 347 | (98.3) | 89 | (100.0) | 87 | (97.8) | 523 | (98.5) |
| Subjects Events | | | | | | | | |
| Subjects Dosed | 350 | (99.2) | 89 | (100) | 88 | (98.9) | 527 | (99.2) |
| Subjects Initiated Surgery | 349 | (98.9) | 89 | (100) | 87 | (97.8) | 525 | (98.9) |
| Subjects Completed Surgery | 349 | (98.9) | 89 | (100) | 87 | (97.8) | 525 | (98.9) |
| End of Study Status | | | | | | | | |
| Completed | 350 | (99.2) | 89 | (100) | 88 | (98.9) | 527 | (99.2) |
| Discontinued | 3 | (0.8) | 0 | | 1 | (1.1) | 4 | (0.8) |
| Primary Reason for Study | | | | | | | | |
| Discontinuation | | | | | | | | |

| Withdrawal by Subject | 1 | (0.3) | 0 | 0 | | 1 | (0.2) |
|---|---|---|---|---|---|---|---|
| Adverse Event | 1 | (0.3) | 0 | 0 | | 1 | (0.2) |
| Pregnancy | 0 | | 0 | 0 | | 0 | |
| Physician Decision | 0 | | 0 | 0 | | 0 | |
| Protocol Violation | 1 | (0.3) | 0 | 1 | (1.1) | 2 | (0.4) |

| | | | | |
|---|---|---|---|---|
| Lost to Follow Up | 0 | 0 | 0 | 0 |
| Other | 0 | 0 | 0 | 0 |

TABLE 40
Summary of Protocol Deviations (ITT Analysis Set)

| | MELT-300 | | Midazolam | | Placebo | | Overall | |
|---|---|---|---|---|---|---|---|---|
| | (N = 353) | | (N = 89) | | (N = 89) | | (N = 541) | |
| | n(%) | | n(%) | | n(%) | | n(%) | |
| Any site-level deviation | | | | | | | 3 | (23.1) |
| Regulatory or ICH-GCP | | | | | | | 0 | |
| Data Privacy | | | | | | | 0 | |
| IP (non-subject) | | | | | | | 0 | |
| Equipment or Facilities | | | | | | | 0 | |
| Training or Qualification | | | | | | | 0 | |
| Safety Reporting | | | | | | | 3 | (23.1) |
| Study Procedure (non-subject) | | | | | | | 0 | |
| Other | | | | | | | 0 | |
| Any subject-level protocol deviation | 202 | (57.2) | 46 | (51.7) | 46 | (51.7) | 294 | (55.4) |
| Inclusion and Exclusion Criteria | 13 | (3.7) | 2 | (2.2) | 19 | (3.6) | 19 | (3.6) |
| Study Visit Schedule | 144 | (40.8) | 30 | (33.7) | 210 | (39.5) | 210 | (39.5) |
| Missed Procedure | 79 | (22.4) | 20 | (22.5) | 117 | (22.0) | 117 | (22.0) |
| Investigational Product | 1 | (0.3) | 0 | | 1 | (0.2) | 1 | (0.2) |
| Informed Consent | 8 | (2.3) | 5 | (5.6) | 16 | (3.0) | 16 | (3.0) |

| | | | | | | |
|---|---|---|---|---|---|---|
| Prohibited Medication | 0 | 0 | 1 | (0.2) | 1 | (0.2) |

| | | | | |
|---|---|---|---|---|
| Other | 0 | 0 | 0 | 0 |

TABLE 41
Summary of Demographics and Baseline
Characteristics (ITT Analysis Set)

| | MELT-300 | | Placebo | | Placebo | |
|---|---|---|---|---|---|---|
| | (N = 353) | | (N = 89) | | (N = 89) | |
| Age (years) | | | | | | |
| n | 353 | | 89 | | 89 | |
| Mean ± SD | 69.1 ± 7.09 | | 68.6 ± 7.99 | | 67.9 ± 8.41 | |
| Median | 70.0 | | 69.0 | | 69.0 | |
| Min, Max | 30, 89 | | 45, 84 | | 36, 85 | |
| Age Group - n (%) | | | | | | |
| 18 to <65 years | 77 | (21.8) | 18 | (20.0) | 25 | (28.1) |
| >=65 years | 276 | (78.2) | 71 | (79.8) | 64 | (71.9) |
| Sex - n (%) | | | | | | |
| 18 to <65 years | 216 | (61.2) | 57 | (64.0) | 53 | (59.6) |
| >=65 years | 137 | (38.8) | 32 | (36.0) | 36 | (40.4) |
| Ethnicity - n (%) | | | | | | |
| 18 to <65 years | 32 | (9.1) | 9 | (10.1) | 9 | (10.1) |
| >=65 years | 321 | (90.9) | 80 | (89.9) | 80 | (89.9) |
| Race - n (%) | | | | | | |
| American Indian or | 1 | (0.3) | 0 | | 1 | (1.1) |
| Alaska Native | | | | | | |
| Asian | 2 | (0.6) | 0 | | 1 | (1.1) |
| Black or African American | 22 | (6.2) | 0 | | 2 | (2.2) |
| Native Hawaiian or | 0 | | 1 | (1.1) | 0 | |
| Other Pacific Islander | | | | | | |
| White | 320 | (90.7) | 88 | (98.9) | 83 | (93.3) |
| Other | 5 | (1.4) | 0 | | 1 | (1.1) |
| Multiple | 1 | (0.3) | 0 | | 1 | (1.1) |
| Unknown | 2 | (0.6) | 0 | | 0 | |
| Caffeine per Day (cups) | | | | | | |
| n | 353 | | 89 | | 89 | |
| Mean ± SD | 1.9 ± 2.00 | | 2.1 ± 1.88 | | 1.9 ± 1.75 | |
| Median | 2.0 | | 2.0 | | 2.0 | |
| Min, Max | 0, 18 | | 0, 10 | | 0, 10 | |
| Smoking History - n (%) | | | | | | |
| Never | 235 | (66.6) | 50 | (56.2) | 52 | (58.4) |
| Former | 80 | (22.7) | 28 | (31.5) | 24 | (27.0) |
| Current | 38 | (10.8) | 11 | (12.4) | 13 | (14.6) |
| History of Illicit Drug or | | | | | | |
| Alcohol Abuse - n (%) | | | | | | |
| Yes | 13 | (3.7) | 8 | (9.0) | 7 | (7.9) |
| No | 340 | (96.3) | 81 | (91.0) | 82 | (92.1) |
| Drug/Alcohol-Free for >=5 | | | | | | |
| Years - n (%) | | | | | | |
| Yes | 13 | (100.0) | 8 | (100.0) | 7 | (100.0) |

| | | | |
|---|---|---|---|
| No | 0 | 0 | 0 |

TABLE 42
Primary Analysis of Procedural Sedation
Responders (ITT Analysis Set)

| | MELT-300 | Midazolam | Placebo |
|---|---|---|---|
| | (N = 353) | (N = 89) | (N = 89) |
| Procedural sedation | 277 (78.5) | 58 (65.2) | 32 (36.0) |
| responder - n (%) | | | |
| MELT-300 vs | | | |
| comparator | | | |
| Main analysis: | | | |
| Pearson's | | | |
| chi-square test: | | | |
| p-value | | 0.009 | <.001 |
| Statistic | | 6.85 | 61.08 |
| Degrees of freedom | | 1 | 1 |
| Risk difference | | 0.133 | 0.425 |
| 95% CI | | (0.031, | (0.313, |
| | | 0.244) | 0.527) |
| Supportive stratified | | | |
| analysis: | | | |
| CMH row-mean | | | |
| score test: | | | |
| p-value | | 0.008 | <.001 |
| Statistic | | 7.06 | 66.38 |
| Degrees of freedom | | 1 | 1 |
| Common risk | | 0.155 | 0.413 |
| difference 95% CI | | (0.060, | (0.323, |
| | | 0.250) | 0.502) |

TABLE 43
Supplementary Analysis of the Primary Endpoint: Procedural
Sedation Responders (All-Treated Analysis Set)

| | MELT-300 | Midazolam | Placebo |
|---|---|---|---|
| | (N = 350) | (N = 89) | (N = 88) |
| Procedural sedation | 277 (79.1) | 58 (65.2) | 32 (36.4) |
| responder - n (%) | | | |
| MELT-300 vs | | | |
| comparator | | | |
| Main analysis: | | | |
| Pearson's | | | |
| chi-square test: | | | |
| p-value | | 0.006 | <.001 |
| Statistic | | 7.66 | 61.94 |
| Degrees of freedom | | 1 | 1 |
| Risk difference | | 0.140 | 0.428 |
| 95% CI | | (0.038, | (0.315, |
| | | 0.251) | 0.530) |
| Supportive stratified | | | |
| analysis: | | | |
| CMH row-mean | | | |
| score test: | | | |
| p-value | | 0.005 | <.001 |
| Statistic | | 7.91 | 67.27 |
| Degrees of freedom | | 1 | 1 |
| Common risk | | 0.161 | 0.413 |
| difference 95% CI | | (0.066, | (0.323, |
| | | 0.256) | 0.504) |

TABLE 44
Supplementary Analysis of the Primary Endpoint:
Procedural Sedation Responders (PP Analysis Set)

| | MELT-300 | Midazolam | Placebo |
|---|---|---|---|
| | (N = 347) | (N = 89) | (N = 87) |
| Procedural sedation responder - n (%) | 275 (79.3) | 58 (65.2) | 32 (36.8) |
| MELT-300 vs comparator | | | |
| Main analysis: | | | |
| Pearson's chi-square test: | | | |
| p-value | | 0.005 | <.001 |
| Statistic | | 7.79 | 60.61 |
| Degrees of freedom | | 1 | 1 |
| Risk difference | | 0.141 | 0.425 |
| 95% CI | | (0.039, 0.252) | (0.311, 0.528) |
| Supportive stratified analysis: | | | |
| CMH row-mean score test: | | | |
| p-value | | 0.004 | <.001 |
| Statistic | | 8.37 | 66.21 |
| Degrees of freedom | | 1 | 1 |
| Common risk difference | | 0.163 | 0.410 |
| 95% CI | | (0.068, 0.258) | (0.319, 0.501) |

TABLE 45
First Key Secondary Analysis of Requirement for
Rescue Sedation Medication (ITT Analysis Set)

| | MELT-300 | Midazolam | Placebo |
|---|---|---|---|
| | (N = 353) | (N = 89) | (N = 89) |
| Requirement for rescue sedation | 68 (19.3) | 30 (33.7) | 55 (61.8) |
| medication - n (%) | | | |
| MELT-300 vs comparator | | | |
| Main analysis: | | | |
| Pearson's chi-square test: | | | |
| p-value | | 0.003 | <.001 |
| Statistic | | 8.59 | 64.03 |
| Degrees of freedom | | 1 | 1 |
| Risk difference | | −0.144 | −0.425 |
| 95% CI | | (−0.255, −0.044) | (−0.528, −0.313) |
| Supportive stratified analysis: | | | |
| CMH row-mean score test: | | | |
| p-value | | 0.003 | <.001 |
| Statistic | | 8.78 | 67.67 |
| Degrees of freedom | | 1 | 1 |
| Common risk difference | | −0.168 | −0.402 |
| 95% CI | | (−0.263, −0.073) | (−0.493, −0.310) |

TABLE 46
Supplementary Analysis of the First Key Secondary Endpoint: Requirement
for Rescue Sedation Medication (All-Treated Analysis Set)

| | MELT-300 | Midazolam | Placebo |
|---|---|---|---|
| | (N = 350) | (N = 89) | (N = 88) |
| Requirement for rescue sedation | 68 (19.4) | 30 (33.7) | 55 (62.5) |
| medication - n (%) | | | |
| MELT-300 vs comparator | | | |
| Main analysis: | | | |
| Pearson's chi-square test: | | | |
| p-value | | 0.004 | <.001 |
| Statistic | | 8.34 | 64.59 |
| Degrees of freedom | | 1 | 1 |
| Risk difference | | −0.143 | −0.431 |
| 95% CI | | (−0.253, −0.042) | (−0.534, −0.318) |
| Supportive stratified analysis: | | | |
| CMH row-mean score test: | | | |
| p-value | | 0.003 | <.001 |
| Statistic | | 8.54 | 68.86 |
| Degrees of freedom | | 1 | 1 |
| Common risk difference | | −0.166 | −0.422 |
| 95% CI | | (−0.261, −0.072) | (−0.512, −0.332) |

TABLE 47
Supplementary Analysis of the First Key Secondary Endpoint:
Requirement for Rescue Sedation Medication (PP Analysis Set)

| | MELT-300 | Midazolam | Placebo |
|---|---|---|---|
| | (N = 347) | (N = 89) | (N = 87) |
| Requirement for rescue sedation | 68 (19.6) | 30 (33.7) | 55 (63.2) |
| medication - n (%) | | | |
| MELT-300 vs comparator | | | |
| Main analysis: | | | |
| Pearson's chi-square test: | | | |
| p-value | | 0.004 | <.001 |
| Statistic | | 8.09 | 65.18 |
| Degrees of freedom | | 1 | 1 |
| Risk difference | | −0.141 | −0.436 |
| 95% CI | | (−0.252, −0.041) | (−0.539, −0.323) |
| Supportive stratified analysis: | | | |
| CMH row-mean score test: | | | |
| p-value | | 0.004 | <.001 |
| Statistic | | 8.36 | 69.73 |
| Degrees of freedom | | 1 | 1 |
| Common risk difference | | −0.165 | −0.424 |
| 95% CI | | (−0.260, −0.070) | (−0.514, −0.334) |

TABLE 48
Second Key Secondary Analysis of Preoperative Procedural
Sedation Responders (ITT Analysis Set)

| | MELT-300 | Midazolam | Placebo |
|---|---|---|---|
| | (N = 353) | (N = 89) | (N = 89) |
| Preoperative procedural sedation | 289 (81.9) | 65 (73.0) | 34 (38.2) |
| responder - n (%) | | | |
| MELT-300 vs comparator | | | |
| Main analysis: | | | |
| Pearson's chi-square test: | | | |
| p-value | | 0.062 | <.001 |
| Statistic | | 3.48 | 68.89 |
| Degrees of freedom | | 1 | 1 |
| Risk difference | | 0.088 | 0.437 |
| 95% CI | | (−0.004, 0.196) | (0.325, 0.539) |
| Supportive stratified analysis: | | | |
| CMH row-mean score test: | | | |
| p-value | | 0.032 | <.001 |
| Statistic | | 4.62 | 75.28 |
| Degrees of freedom | | 1 | 1 |
| Common risk difference | | 0.126 | 0.432 |
| 95% CI | | (0.034, 0.218) | (0.343, 0.520) |

TABLE 49
Supplementary Analysis of the Second Key Secondary Endpoint: Preoperative
Procedural Sedation Responders (All-Treated Analysis Set)

| | MELT-300 | Midazolam | Placebo |
|---|---|---|---|
| | (N = 350) | (N = 89) | (N = 88) |
| Preoperative procedural sedation | 289 (82.6) | 65 (73.0) | 34 (38.6) |
| responder - n (%) | | | |
| MELT-300 vs comparator | | | |
| Main analysis: | | | |
| Pearson's chi-square test: | | | |
| p-value | | 0.042 | <.001 |
| Statistic | | 4.13 | 70.10 |
| Degrees of freedom | | 1 | 1 |
| Risk difference | | 0.095 | 0.439 |
| 95% CI | | (0.003, 0.203) | (0.327, 0.542) |
| Supportive stratified analysis: | | | |
| CMH row-mean score test: | | | |
| p-value | | 0.021 | <.001 |
| Statistic | | 5.34 | 76.78 |
| Degrees of freedom | | 1 | 1 |
| Common risk difference | | 0.132 | 0.432 |
| 95% CI | | (0.041, 0.224) | (0.343, 0.522) |

TABLE 50
Supplementary Analysis of the Second Key Secondary Endpoint:
Preoperative Procedural Sedation Responders (PP Analysis Set)

| | MELT-300 | Midazolam | Placebo |
|---|---|---|---|
| | (N = 347) | (N = 89) | (N = 87) |
| Preoperative procedural sedation | 287 (82.7) | 65 (73.0) | 34 (39.1) |
| responder - n (%) | | | |
| MELT-300 vs comparator | | | |
| Main analysis: | | | |
| Pearson's chi-square test: | | | |
| p-value | | 0.039 | <.001 |
| Statistic | | 4.26 | 68.75 |
| Degrees of freedom | | 1 | 1 |
| Risk difference | | 0.097 | 0.436 |
| 95% CI | | (0.004, 0.204) | (0.323, 0.540) |
| Supportive stratified analysis: | | | |
| CMH row-mean score test: | | | |
| p-value | | 0.016 | <.001 |
| Statistic | | 5.75 | 75.87 |
| Degrees of freedom | | 1 | 1 |
| Common risk difference | | 0.134 | 0.430 |
| 95% CI | | (0.043, 0.226) | (0.340, 0.519) |

TABLE 51
Secondary Analysis of Rescue Sedation Medication Use (ITT Analysis Set)

| | MELT-300 | Midazolam | Placebo |
|---|---|---|---|
| | (N = 353) | (N = 89) | (N = 89) |
| Received preoperative rescue sedation | 37 (10.5) | 16 (18.0) | 28 (31.5) |
| medication - n (%) | | | |
| MELT-300 vs comparator | | | |
| Pearson's chi-square test: | | | |
| p-value | | 0.052 | <.001 |
| Statistic | | 3.78 | 24.94 |
| Degrees of freedom | | 1 | 1 |
| Received intraoperative rescue sedation | 30 (8.5) | 15 (16.9) | 28 (31.5) |
| medication - n (%) | | | |
| MELT-300 vs comparator | | | |
| Pearson's chi-square test: | | | |
| p-value | | 0.020 | <.001 |
| Statistic | | 5.43 | 32.87 |
| Degrees of freedom | | 1 | 1 |

TABLE 52
Secondary Analysis of Ramsay Sedation Scale Scores (ITT Analysis Set)

| Visit | MELT-300 | | Midazolam | | Placebo | |
|---|---|---|---|---|---|---|
| Score | (N = 353) | | (N = 89) | | (N = 89) | |
| Baseline | | | | | | |
| Prior rescue sedation medication given | 0 | | 0 | | 0 | |
| 1 | 350 | (99.2) | 89 | (100.0) | 88 | (98.9) |
| 2-3 | 0 | | 0 | | 0 | |
| 4 | 0 | | 0 | | 0 | |
| 5+ | 0 | | 0 | | 0 | |
| Missing | 3 | (0.8) | 0 | | 1 | (1.1) |
| MELT-300 vs comparator | | | | | | |
| CMH row-mean score test: | | | | | | |
| p-value | | | — | | — | |
| Statistic | | | — | | — | |
| Degrees of freedom | | | — | | — | |
| Just prior to the start of surgery | | | | | | |
| Prior rescue sedation medication given | 36 | (10.2) | 17 | (19.1) | 28 | (31.5) |
| 1 | 20 | (5.7) | 7 | (7.9) | 25 | (28.1) |
| 2-3 | 290 | (82.2) | 65 | (73.0) | 34 | (38.2) |
| 4 | 3 | (0.8) | 0 | | 0 | |
| 5+ | 0 | | 0 | | 0 | |
| Missing | 4 | (1.1) | 0 | | 2 | (2.2) |
| MELT-300 vs comparator | | | | | | |
| CMH row-mean score test: | | | | | | |
| p-value | | | 0.006 | | <.001 | |
| Statistic | | | 7.58 | | 75.89 | |
| Degrees of freedom | | | 1 | | 1 | |
| Maximum preoperative score | | | | | | |
| Prior rescue sedation medication given | 0 | | 0 | | 0 | |
| 1 | 53 | (15.0) | 22 | (24.7) | 51 | (57.3) |
| 2-3 | 294 | (83.3) | 67 | (75.3) | 37 | (41.6) |
| 4 | 3 | (0.8) | 0 | | 0 | |
| 5+ | 0 | | 0 | | 0 | |
| Missing | 3 | (0.8) | 0 | | 1 | (1.1) |
| MELT-300 vs comparator | | | | | | |
| CMH row-mean score test: | | | | | | |
| p-value | | | 0.015 | | <.001 | |
| Statistic | | | 5.89 | | 76.77 | |
| Degrees of freedom | | | 1 | | 1 | |
| Minimum intraoperative score | | | | | | |
| Prior rescue sedation medication given | 46 | (13.0) | 25 | (28.1) | 37 | (41.6) |
| 1 | 17 | (4.8) | 4 | (4.5) | 17 | (19.1) |
| 2-3 | 284 | (80.5) | 60 | (67.4) | 33 | (37.1) |
| 4 | 2 | (0.6) | 0 | | 0 | |
| 5+ | 0 | | 0 | | 0 | |
| Missing | 4 | (1.1) | 0 | | 2 | (2.2) |
| MELT-300 vs comparator | | | | | | |
| CMH row-mean score test: | | | | | | |
| p-value | | | <.001 | | <.001 | |
| Statistic | | | 11.11 | | 74.26 | |
| Degrees of freedom | | | 1 | | 1 | |
| Maximum intraoperative score | | | | | | |
| Prior rescue sedation medication given | 46 | (13.0) | 25 | (28.1) | 37 | (41.6) |
| 1 | 11 | (3.1) | 4 | (4.5) | 12 | (13.5) |
| 2-3 | 289 | (81.9) | 60 | (67.4) | 38 | (42.7) |
| 4 | 3 | (0.8) | 0 | | 0 | |
| 5+ | 0 | | 0 | | 0 | |
| Missing | 4 | (1.1) | 0 | | 2 | (2.2) |
| MELT-300 vs comparator | | | | | | |
| CMH row-mean score test: | | | | | | |
| p-value | | | <.001 | | <.001 | |
| Statistic | | | 14.39 | | 69.35 | |
| Degrees of freedom | | | 1 | | 1 | |
| Minimum postoperative score | | | | | | |
| Prior rescue sedation medication given | 66 | (18.7) | 29 | (32.6) | 55 | (61.8) |
| 1 | 214 | (60.6) | 49 | (55.1) | 28 | (31.5) |
| 2-3 | 66 | (18.7) | 10 | (11.2) | 4 | (4.5) |
| 4 | 0 | | 0 | | 0 | |
| 5+ | 0 | | 0 | | 0 | |
| Missing | 7 | (2.0) | 1 | (1.1) | 2 | (2.2) |
| MELT-300 vs comparator | | | | | | |
| CMH row-mean score test: | | | | | | |
| p-value | | | 0.002 | | <.001 | |
| Statistic | | | 9.68 | | 62.42 | |
| Degrees of freedom | | | 1 | | 1 | |
| Maximum postoperative score | | | | | | |
| Prior rescue sedation medication given | 66 | (18.7) | 29 | (32.6) | 55 | (61.8) |
| 1 | 177 | (50.1) | 45 | (50.6) | 27 | (30.3) |
| 2-3 | 103 | (29.2) | 14 | (15.7) | 5 | (5.6) |
| 4 | 0 | | 0 | | 0 | |
| 5+ | 0 | | 0 | | 0 | |
| Missing | 7 | (2.0) | 1 | (1.1) | 2 | (2.2) |
| MELT-300 vs comparator | | | | | | |
| CMH row-mean score test: | | | | | | |
| p-value | | | 0.001 | | <.001 | |
| Statistic | | | 10.67 | | 63.60 | |
| Degrees of freedom | | | 1 | | 1 | |
| Maximum score overall | | | | | | |
| Prior rescue sedation medication given | 0 | | 0 | | 0 | |
| 1 | 51 | (14.4) | 22 | (24.7) | 47 | (52.8) |
| 2-3 | 296 | (83.9) | 67 | (75.3) | 41 | (46.1) |

| 4 | 3 | (0.8) | 0 | 0 | |
|---|---|---|---|---|---|
| 5+ | 0 | | 0 | 0 | |
| Missing | 3 | (0.8) | 0 | 1 | (1.1) |

| | | |
|---|---|---|
| MELT-300 vs comparator | | |
| CMH row-mean score test: | | |
| p-value | 0.009 | <.001 |
| Statistic | 6.77 | 71.59 |
| Degrees of freedom | 1 | 1 |

TABLE 53
Evaluation of Time to Achieve Preoperative Target
Sedation Level (All-Treated Analysis Set)

| | MELT-300 | |
|---|---|---|
| | (N = 353) | |
| Time to achieve preoperative target sedation level | | |
| Subject Events - n (%) | | |
| Achieved preoperative target sedation level | 293 | (83.0) |
| KM time-to-event estimates (minutes) (95% CI): | | |
| Q1 | 10 | (NE, NE) |
| Median | 15 | (10, 15) |
| Q3 | 15 | (NE, NE) |
| KM event-free survival rates (95% CI): | | |
| 5 Minutes | 0.94 | (0.91, 0.96) |
| 10 Minutes | 0.53 | (0.47, 0.59) |
| 15 Minutes | 0.14 | (0.11, 0.19) |
| 20 Minutes | 0.03 | (0.02, 0.06) |
| 25 Minutes | 0.00 | (0.00, 0.02) |
| 30 Minutes | 0.00 | (NE, NE) |
| Sensitivity analysis of time to achieve | | |
| preoperative target sedation level | | |
| Subject Events - n (%) | | |
| Achieved preoperative target sedation level | 293 | (83.0) |
| Censored | 37 | (10.5) |
| KM time-to-event estimates (minutes) (95% CI): | | |
| Q1 | 10 | (NE, NE) |
| Median | 15 | (NE, NE) |
| Q3 | 15 | (15, 20) |
| KM event-free survival rates (95% CI): | | |
| 5 Minutes | 0.95 | (0.92, 0.97) |
| 10 Minutes | 0.58 | (0.53, 0.63) |
| 15 Minutes | 0.23 | (0.19, 0.28) |
| 20 Minutes | 0.14 | (0.10, 0.18) |
| 25 Minutes | 0.06 | (0.03, 0.10) |
| 30 Minutes | 0.03 | (0.00, 0.10) |
| 35 Minutes | NE | (NE, NE) |

TABLE 54
Secondary Analysis of Surgery Completion (ITT Analysis Set)

| | MELT-300 | Midazolam | Placebo |
|---|---|---|---|
| | (N = 353) | (N = 89) | (N = 89) |
| Completed the surgery - n (%) | 349 (98.9) | 89 (100.0) | 87 (97.8) |
| MELT-300 vs comparator | | | |
| Fisher's exact test: | | | |
| p-value | | 0.588 | 0.348 |
| Completed surgery without intervention | 349 (98.9) | 89 (100.0) | 87 (97.8) |
| due to pain or anxiety - n (%) | | | |
| MELT-300 vs comparator | | | |
| Fisher's exact test: | | | |
| p-value | | 0.588 | 0.348 |

TABLE 55
Analysis of Surgeon Satisfaction (ITT Analysis Set)

| | MELT-300 | | Midazolam | | Placebo | |
|---|---|---|---|---|---|---|
| | (N = 353) | | (N = 89) | | (N = 89) | |
| Surgeon Satisfaction Scores | | | | | | |
| N | 349 | | 89 | | 87 | |
| Mean ± SD | 4.8 ± 0.49 | | 4.8 ± 0.56 | | 4.7 ± 0.67 | |
| Median | 5.0 | | 5.0 | | 5.0 | |
| Min, Max | 2, 5 | | 2, 5 | | 2, 5 | |
| MELT-300 vs comparator | | | | | | |
| 2-Sample t-test: | | | | | | |
| Difference (95% CI) | | | 0.02 | (−0.11, 0.15) | 0.12 | (−0.04, 0.27) |
| p-value | | | 0.777 | | 0.135 | |
| T-statistic | | | 0.28 | | 1.50 | |
| Degrees of freedom | | | 124 | | 110 | |
| Surgeon Satisfaction Scores - n (%) | | | | | | |
| 5 = Very Satisfied | 288 | (81.6) | 74 | (83.1) | 66 | (74.2) |
| 4 = Satisfied | 52 | (14.7) | 11 | (12.4) | 17 | (19.1) |
| 3 = Neither Satisfied Nor Dissatisfied | 7 | (2.0) | 3 | (3.4) | 1 | (1.1) |
| 2 = Dissatisfied | 2 | (0.6) | 1 | (1.1) | 3 | (3.4) |

| 1 = Very Dissatisfied | 0 | 0 | 0 |
|---|---|---|---|
| MELT-300 vs comparator | | | |
| Fisher's Exact Test: | | | |
| p-value | | 0.310 | 0.302 |

TABLE 56
Analysis of Patient Satisfaction (ITT Analysis Set)

| | MELT-300 | | Midazolam | | Placebo | |
|---|---|---|---|---|---|---|
| | (N = 353) | | (N = 89) | | (N = 89) | |
| Patient Satisfaction Scores | | | | | | |
| N | 350 | | 89 | | 88 | |
| Mean ± SD | 4.8 ± 0.53 | | 4.8 ± 0.55 | | 4.6 ± 0.71 | |
| Median | 5.0 | | 5.0 | | 5.0 | |
| Min, Max | 1, 5 | | 2, 5 | | 1, 5 | |
| MELT-300 vs comparator | | | | | | |
| 2-Sample t-test: | | | | | | |
| Difference (95% CI) | | | 0.02 | (−0.10, 0.15) | 0.19 | (0.03, 0.35) |
| p-value | | | 0.707 | | 0.022 | |
| T-statistic | | | 0.38 | | 2.32 | |
| Degrees of freedom | | | 132 | | 113 | |
| Patient Satisfaction Scores - n (%) | | | | | | |
| 5 = Very Satisfied | 284 | (80.5) | 71 | (79.8) | 59 | (66.3) |
| 4 = Satisfied | 59 | (16.7) | 15 | (16.9) | 25 | (28.1) |
| 3 = Neither Satisfied Nor Dissatisfied | 3 | (0.8) | 2 | (2.2) | 2 | (2.2) |
| 2 = Dissatisfied | 3 | (0.8) | 1 | (1.1) | 1 | (1.1) |

| | | | | | |
|---|---|---|---|---|---|
| 1 = Very Dissatisfied | 1 | (0.3) | 0 | 1 | (1.1) |

| | | |
|---|---|---|
| MELT-300 vs comparator | | |
| Fisher's Exact Test: | | |
| p-value | 0.431 | 0.243 |

TABLE 57
Overview of Adverse Events (Safety Analysis Set)

| | Melt | | | | | | | |
|---|---|---|---|---|---|---|---|---|
| | 300 | | Midazolam | | Placebo | | Overall | |
| | (N = 350) | | (N = 89) | | (N = 88) | | (N = 527) | |
| | n (%) | # | n (%) | # | n (%) | # | n (%) | # |
| Location: Overall | | | | | | | | |
| Any AE | 72 (20.6) | 112 | 16 (18.0) | 24 | 16 (18.2) | 4 | 104 (19.7) | 160 |
| Any TEAE | 68 (19.4) | 96 | 13 (14.6) | 20 | 15 (17.0) | 2 | 96 (18.2) | 138 |
| Any Treatment-Related TEAE | 25 (7.1) | 32 | 2 (2.2) | 4 | 4 (4.5) | 7 | 31 (5.9) | 43 |
| Any TEAESI | 32 (9.1) | 34 | 2 (2.2) | 2 | 7 (8.0) | 9 | 41 (7.8) | 45 |
| Ketamine emergence | 0 | 0 | 0 | 0 | 0 | 0 | 0 | 0 |
| delirium | | | | | | | | |
| Respiratory AEs | 10 (2.9) | 10 | 1 (1.1) | 1 | 1 (1.1) | 1 | 12 (2.3) | 12 |
| Cardiovascular AEs | 13 (3.7) | 14 | 1 (1.1) | 1 | 5 (5.7) | 6 | 19 (3.6) | 21 |
| Neurocognitive AEs | 1 (0.3) | 1 | 0 | 0 | 0 | 0 | 1 (0.2) | 1 |
| Abuse-related AEs | 7 (2.0) | 8 | 0 | 0 | 1 (1.1) | 1 | 8 (1.5) | 9 |
| Oversedation requiring the | 0 | 0 | 0 | 0 | 0 | 0 | 0 | 0 |
| use of flumazenil | | | | | | | | |
| Oral/pharyngeal AEs | 2 (0.6) | 2 | 0 | 0 | 1 (1.1) | 1 | 3 (0.6) | 3 |
| Any Treatment-Related | 14 (4.0) | 15 | 1 (1.1) | 1 | 4 (4.5) | 5 | 19 (3.6) | 21 |
| TEAESI | | | | | | | | |
| Any Grade 3+ TEAE | 1 (0.3) | 1 | 0 | 0 | 0 | 0 | 1 (0.2) | 1 |
| Any Grade 3+ Treatment- | 0 | 0 | | | | | | |
| Related TEAE | | | | | | | | |
| Any SAE | 0 | 0 | 0 | 0 | 0 | 0 | 0 | 0 |
| Any TESAE | 0 | 0 | 0 | 0 | 0 | 0 | 0 | 0 |
| Any Treatment-Related TESAE | 0 | 0 | 0 | 0 | 0 | 0 | 0 | 0 |
| Any Unexpected TEAE | 24 (6.9) | 28 | 6 (6.7) | 9 | 6 (6.8) | 6 | 36 (6.8) | 43 |
| Any Unexpected Treatment- | 0 | 0 | 0 | 0 | 0 | 0 | 0 | 0 |
| Related TEAE | | | | | | | | |
| Any TEAE Resulting in Study | 0 | 0 | 0 | 0 | 0 | 0 | 0 | 0 |
| Drug Interruption | | | | | | | | |
| Any TEAE Resulting in Study | 0 | 0 | 0 | 0 | 0 | 0 | 0 | 0 |
| Drug Withdrawal | | | | | | | | |
| Any TEAE Resulting in Death | 0 | 0 | 0 | 0 | 0 | 0 | 0 | 0 |
| Any Treatment-Related TEAE | 0 | 0 | 0 | 0 | 0 | 0 | 0 | 0 |
| Resulting in Death | | | | | | | | |
| Location: Non-Ocular | | | | | | | | |
| Any AE | 53 (15.1) | 80 | 7 (7.9) | 9 | 9 (10.2) | 14 | 69 (13.1) | 103 |
| Any TEAE | 48 (13.7) | 64 | 4 (4.5) | 5 | 8 (9.1) | 12 | 60 (11.4) | 81 |
| Any Treatment-Related TEAE | 24 (6.9) | 31 | 2 (2.2) | 3 | 4 (4.5) | 7 | 30 (5.7) | 41 |
| Any TEAESI | 32 (9.1) | 34 | 2 (2.2) | 2 | 7 (8.0) | 9 | 41 (7.8) | 45 |
| Ketamine emergence delirium | 0 | 0 | 0 | 0 | 0 | 0 | 0 | 0 |
| Respiratory AEs | 10 (2.9) | 10 | 1 (1.1) | 1 | 1 (1.1) | 1 | 12 (2.3) | 12 |
| Cardiovascular AEs | 13 (3.7) | 14 | 1 (1.1) | 1 | 5 (5.7) | 6 | 19 (3.6) | 21 |
| Neurocognitive AEs | 1 (0.3) | 1 | 0 | 0 | 0 | 0 | 1 (0.2) | 1 |
| Abuse-related AEs | 7 (2.0) | 8 | 0 | 0 | 1 (1.1) | 1 | 8 (1.5) | 9 |
| Oversedation requiring the use | 0 | 0 | 0 | 0 | 0 | 0 | 0 | 0 |
| of flumazenil | | | | | | | | |
| Oral/pharyngeal AEs | 2 (0.6) | 2 | 0 | 0 | 1 (1.1) | 1 | 3 (0.6) | 3 |
| Any Treatment-Related TEAESI | 14 (4.0) | 15 | 1 (1.1) | 1 | 4 (4.5) | 5 | 19 (3.6) | 21 |
| Any Grade 3+ TEAE | 0 | 0 | 0 | 0 | 0 | 0 | 0 | 0 |
| Any Grade 3+ Treatment- | 0 | 0 | | | | | | |
| Related TEAE | | | | | | | | |
| Any SAE | 0 | 0 | 0 | 0 | 0 | 0 | 0 | 0 |
| Any TESAE | 0 | 0 | 0 | 0 | 0 | 0 | 0 | 0 |
| Any Treatment-Related TESAE | 0 | 0 | 0 | 0 | 0 | 0 | 0 | 0 |
| Any Unexpected TEAE | 6 (1.7) | 6 | 0 | 0 | 0 | 0 | 6 (1.1) | 6 |
| Any Unexpected Treatment- | 0 | 0 | 0 | 0 | 0 | 0 | 0 | 0 |
| Related TEAE | | | | | | | | |
| Any TEAE Resulting in Study | 0 | 0 | 0 | 0 | 0 | 0 | 0 | 0 |
| Drug Interruption | | | | | | | | |
| Any TEAE Resulting in Study | 0 | 0 | 0 | 0 | 0 | 0 | 0 | 0 |
| Drug Withdrawal | | | | | | | | |
| Any TEAE Resulting in Death | 0 | 0 | 0 | 0 | 0 | 0 | 0 | 0 |
| Any Treatment-Related TEAE | 0 | 0 | 0 | 0 | 0 | 0 | 0 | 0 |
| Resulting in Death | | | | | | | | |
| Location: Ocular | | | | | | | | |
| Any AE | 26 (7.4) | 32 | 10 (11.2) | 15 | 9 (10.2) | 10 | 45 (8.5) | 57 |
| Any TEAE | 26 (7.4) | 32 | 10 (11.2) | 15 | 9 (10.2) | 10 | 45 (8.5) | 57 |
| Any Treatment-Related TEAE | 1 (0.3) | 1 | 1 (1.1) | 1 | 0 | 0 | 2 (0.4) | 2 |
| Any TEAESI | 0 | 0 | 2 (2.2) | 0 | 0 | 0 | 0 | 0 |
| Ketamine emergence delirium | 0 | 0 | 0 | 0 | 0 | 0 | 0 | 0 |
| Respiratory AEs | 0 | 0 | 1 (1.1) | 0 | 0 | 0 | 0 | 0 |
| Cardiovascular AEs | 0 | 0 | 1 (1.1) | 0 | 0 | 0 | 0 | 0 |
| Neurocognitive AEs | 0 | 0 | 0 | 0 | 0 | 0 | 0 | 0 |
| Abuse-related AEs | 0 | 0 | 0 | 0 | 0 | 0 | 0 | 0 |
| Oversedation requiring the use | 0 | 0 | 0 | 0 | 0 | 0 | 0 | 0 |
| of flumazenil | | | | | | | | |
| Oral/pharyngeal AEs | 0 | 0 | 0 | 0 | 0 | 0 | 0 | 0 |
| Any Treatment-Related TEAESI | 0 | 0 | 0 | 0 | 0 | 0 | 0 | 0 |
| Any Grade 3+ TEAE | 1 (0.3) | 1 | 0 | 0 | | | 1 (0.2) | 1 |
| Any Grade 3+ Treatment- | 0 | 0 | 0 | 0 | 0 | 0 | 0 | 0 |
| Related TEAE | | | | | | | | |
| Any SAE | 0 | 0 | 0 | 0 | 0 | 0 | 0 | 0 |
| Any TESAE | 0 | 0 | 0 | 0 | 0 | 0 | 0 | 0 |
| Any Treatment-Related TESAE | 0 | 0 | 0 | 0 | 0 | 0 | 0 | 0 |
| Any Unexpected TEAE | 19 (5.4) | 22 | 6 (6.7) | 9 | 6 (6.8) | 6 | 31 (5.9) | 37 |
| Any Unexpected Treatment- | 0 | 0 | 0 | 0 | 0 | 0 | 0 | 0 |
| Related TEAE | | | | | | | | |
| Any TEAE Resulting in Study | 0 | | 0 | 0 | 0 | 0 | 0 | 0 |
| Drug Interruption | | | | | | | | |
| Any TEAE Resulting in Study | 0 | 0 | 0 | 0 | 0 | 0 | 0 | 0 |
| Drug Withdrawal | | | | | | | | |
| Any TEAE Resulting in Death | 0 | 0 | 0 | 0 | 0 | 0 | 0 | 0 |

TABLE 58
Summary of Treatment-Emergent Adverse Events by Location, System
Organ Class, and Preferred Term (Safety Analysis Set)

| | MELT-300 | | Midazolam | | Placebo | | Overall | |
|---|---|---|---|---|---|---|---|---|
| System Organ Class | (N = 350) | | (N = 89) | | (N = 88) | | (N = 527) | |
| Preferred Term | n(%) | | n(%) | | n( %) | | n(%) | |
| Location: Overall | | | | | | | | |
| Any TEAE | 68 | (19.4) | 13 | (14.6) | 15 | (17.0) | 96 | (18.2) |
| Cardiac disorders | 4 | (1.1) | 1 | (1.1) | 0 | | 5 | (0.9) |
| Tachycardia | 2 | (0.6) | 1 | (1.1) | 0 | | 3 | (0.6) |
| Bradycardia | 2 | (0.6) | 0 | | 0 | | 2 | (0.4) |
| Eye disorders | 24 | (6.9) | 9 | (10.1) | 9 | (10.2) | 42 | (8.0) |
| Vision blurred | 7 | (2.0) | 5 | (5.6) | 4 | (4.5) | 16 | (0.3) |
| Foreign body sensation in eyes | 5 | (1.4) | 2 | (2.2) | 1 | (1.1) | 8 | (1.5) |
| Eye irritation | 2 | (0.6) | 2 | (2.2) | 2 | (2.3) | 6 | (1.1) |
| Eye pain | 2 | (0.6) | 2 | (2.2) | 2 | (2.3) | 6 | (1.1) |
| Halo vision | 1 | (0.3) | 1 | (1.1) | 0 | | 2 | (0.4) |
| Photophobia | 2 | (0.6) | 0 | | 0 | | 2 | (0.4) |
| Photopsia | 2 | (0.6) | 0 | | 0 | | 2 | (0.4) |
| Visual impairment | 2 | (0.6) | 0 | | 0 | | 2 | (0.4) |
| Abnormal sensation in eye | 1 | (0.3) | 0 | | 0 | | 1 | (0.2) |
| Corneal oedema | 1 | (0.3) | 0 | | 0 | | 1 | (0.2) |
| Diplopia | 1 | (0.3) | 0 | | 0 | | 1 | (0.2) |
| Eye discharge | 0 | | 1 | (1.1) | 0 | | 1 | (0.2) |
| Glare | 1 | (0.3) | 0 | | 0 | | 1 | (0.2) |
| Lacrimation increased | 0 | | 0 | | 1 | (1.1) | 1 | (0.2) |
| Ocular discomfort | 1 | (0.3) | 0 | | 0 | | 1 | (0.2) |
| Periorbital discomfort | 0 | | 1 | (1.1) | 0 | | 1 | (0.2) |
| Visual field defect | 1 | (0.3) | 0 | | 0 | | 1 | (0.2) |
| Gastrointestinal disorders | 4 | (1.1) | 0 | | 1 | (1.1) | 5 | (0.9) |
| Nausea | 3 | (0.9) | 0 | | 0 | | 3 | (0.6) |
| Hypoaesthesia oral | 1 | (0.3) | 0 | | 1 | (1.1) | 2 | (0.4) |
| General disorders and administration | | | | | | | | |
| site conditions | | | | | | | | |
| Fatigue | 4 | (1.1) | 0 | | 0 | | 4 | (0.8) |
| Instillation site pain | 2 | (0.6) | 0 | | 0 | | 2 | (0.4) |
| Pyrexia | 1 | (0.3) | 0 | | 0 | | 1 | (0.2) |
| Infections and infestations | 1 | (0.3) | 0 | | 0 | | 1 | (0.2) |
| Sinusitis | 1 | (0.3) | 0 | | 0 | | 1 | (0.2) |
| Injury, poisoning and procedural | 7 | (2.0) | 0 | | 0 | | 7 | (1.3) |
| complications | | | | | | | | |
| Procedural nausea | 4 | (1.1) | 0 | | 0 | | 4 | (0.8) |
| Procedural vomiting | 2 | (0.6) | 0 | | 0 | | 2 | (0.4) |
| Corneal abrasion | 1 | (0.3) | 0 | | 0 | | 1 | (0.2) |
| Post procedural hypotension | 1 | (0.3) | 0 | | 0 | | 1 | (0.2) |
| Procedural pain | 1 | (0.3) | 0 | | 0 | | 1 | (0.2) |
| Investigations | 16 | (4.6) | 0 | | 5 | (5.7) | 21 | (4.0) |
| Blood pressure increased | 7 | (2.0) | 0 | | 0 | | 11 | (2.1) |
| Oxygen saturation decreased | 8 | (2.3) | 0 | | 0 | | 9 | (1.7) |
| Blood pressure systolic increased | 1 | (0.3) | 0 | | 0 | | 1 | (0.2) |
| Musculoskeletal and connective tissue | 3 | (0.9) | 0 | | 0 | | 3 | (0.6) |
| disorders | | | | | | | | |
| Back pain | 1 | (0.3) | 0 | | 0 | | 1 | (0.2) |
| Myalgia | 1 | (0.3) | 0 | | 0 | | 1 | (0.2) |
| Neck pain | 1 | (0.3) | 0 | | 0 | | 1 | (0.2) |
| Nervous system disorders | 11 | (3.1) | 3 | (3.4) | 2 | (2.3) | 16 | (3.0) |
| Headache | 6 | (1.7) | 2 | (2.2) | 2 | (2.3) | 10 | (1.9) |
| Dizziness | 2 | (0.6) | 0 | | 0 | | 2 | (0.4) |
| Amnesia | 1 | (0.3) | 0 | | 0 | | 1 | (0.2) |
| Balance disorder | 1 | (0.3) | 0 | | 0 | | 1 | (0.2) |
| Lethargy | 1 | (0.3) | 0 | | 0 | | 1 | (0.2) |
| Nystagmus | 0 | | 1 | (1.1) | 0 | | 1 | (0.2) |
| Paraesthesia | 0 | | 1 | (1.1) | 0 | | 1 | (0.2) |
| Taste disorder | 1 | (0.3) | 0 | | 0 | | 1 | (0.2) |
| Product issues | 6 | (1.7) | 0 | | 0 | | 6 | (1.1) |
| Product after taste | 6 | (1.7) | 0 | | 0 | | 6 | (1.1) |
| Psychiatric disorders | 2 | (0.6) | 0 | | 1 | (1.1) | 3 | (0.6) |
| Anxiety | 2 | (0.6) | 0 | | 1 | (1.1) | 3 | (0.6) |
| Respiratory, thoracic, and mediastinal | 4 | (1.1) | 1 | (1.1) | 1 | (1.1) | 6 | (1.1) |
| disorders | | | | | | | | |
| Hypoxia | 2 | (0.6) | 1 | (1.1) | 0 | | 3 | (0.6) |
| Cough | 1 | (0.3) | 0 | | 0 | | 1 | (0.2) |
| Hiccups | 0 | | 0 | | 1 | (1.1) | 1 | (0.2) |
| Nasal Discomfort | 1 | (0.3) | 0 | | 0 | | 1 | (0.2) |
| Surgical and medical procedures | 1 | (0.3) | 0 | | 0 | | 1 | (0.2) |
| Dental local anaesthesia | 1 | (0.3) | 0 | | 0 | | 1 | (0.2) |
| Vascular disorders | 0 | | 0 | | 1 | (1.1) | 1 | (0.2) |
| Hypertension | 0 | | 0 | | 1 | (1.1) | 1 | (0.2) |
| Location: Non-Ocular | | | | | | | | |
| Any TEAE | 48 | (13.7) | 4 | (4.5) | 8 | (9.1) | 60 | (11.4) |
| Cardiac disorders | 4 | (1.1) | 0 | | 1 | (1.1) | 5 | (0.9) |
| Tachycardia | 3 | (0.9) | 0 | | 0 | | 3 | (0.6) |
| Bradycardia | 1 | (0.3) | 0 | | 1 | (1.1) | 2 | (0.4) |
| Gastrointestinal disorders | 3 | (0.9) | 0 | | 0 | | | |
| Nausea | 2 | (0.6) | 0 | | 0 | | | |
| Hypoaesthesia oral | 1 | (0.3) | 0 | | 0 | | | |
| General disorders and administration | 3 | (0.9) | 0 | | 0 | | 3 | (0.6) |
| site conditions | | | | | | | | |
| Fatigue | 2 | (0.6) | 0 | | 0 | | 2 | (0.4) |
| Pyrexia | 1 | (0.3) | 0 | | 0 | | 1 | (0.2) |
| Infections and infestations | 1 | (0.3) | 0 | | 0 | | 1 | (0.2) |
| Sinusitis | 1 | (0.3) | 0 | | 0 | | 1 | (0.2) |
| Injury, poisoning and procedural | 5 | (1.4) | 0 | | 0 | | 5 | (0.9) |
| complications | | | | | | | | |
| Procedural nausea | 4 | (1.1) | 0 | | 0 | | 4 | (0.8) |
| Procedural vomiting | 2 | (0.6) | 0 | | 0 | | 2 | (0.4) |
| Post procedural hypotension | 1 | (0.3) | 0 | | 0 | | 1 | (0.2) |
| Investigations | 16 | (4.6) | 0 | | 5 | (5.7) | 5 | (0.9) |
| Blood pressure increased | 7 | (2.) | 0 | | 4 | (4.5) | 4 | (0.8) |
| Oxygen saturation decreased | 8 | (2.3) | 0 | | 1 | (1.1)0 | 2 | (0.4) |
| Blood pressure systolic increased | 1 | (0.3) | 0 | | 0 | | 1 | (0.2) |
| Musculoskeletal and connective tissue | 3 | (0.9) | 0 | | 0 | | 3 | (0.6) |
| disorders | | | | | | | | |
| Back pain | 1 | (0.3) | 0 | | 0 | | 1 | (0.2) |
| Myalgia | 1 | (0.3) | 0 | | 0 | | 1 | (0.2) |
| Neck pain | 1 | (0.3) | 0 | | 0 | | 1 | (0.2) |
| Nervous system disorders | 11 | (3.1) | 3 | (3.4) | 2 | (2.3) | 16 | (3.0) |
| Headache | 6 | (1.7) | 2 | (2.2) | 2 | (2.3) | 10 | (1.9) |
| Dizziness | 2 | (0.6) | 0 | | 0 | | 2 | (0.4) |
| Amnesia | 1 | (0.3) | 0 | | 0 | | 1 | (0.2) |
| Balance disorder | 1 | (0.3) | 0 | | 0 | | 1 | (0.2) |
| Lethargy | 1 | (0.3) | 0 | | 0 | | 1 | (0.2) |
| Paraesthesia | 0 | | 1 | (1.1) | 0 | | 1 | (0.2) |
| Taste disorder | 1 | (0.3) | 0 | | 0 | | 1 | (0.2) |
| Product issues | 6 | (1.7) | 0 | | 0 | | 6 | (1.1) |
| Product after taste | 6 | (1.7) | 0 | | 0 | | 6 | (1.1) |
| Psychiatric disorders | 2 | (0.6) | 0 | | 1 | (1.1) | 3 | (0.6) |
| Anxiety | 2 | (0.6) | 0 | | 1 | (1.1) | 3 | (0.6) |
| Respiratory, thoracic, and mediastinal | 4 | (1.1) | 1 | (1.1) | 1 | (1.1) | 6 | (1.1) |
| disorders | | | | | | | | |
| Hypoxia | 2 | (0.6) | 1 | (1.1) | 0 | | 3 | (0.6) |
| Cough | 1 | (0.3) | 0 | | 0 | | 1 | (0.2) |
| Hiccups | 0 | | 0 | | 1 | (1.1) | 1 | (0.2) |
| Nasal Discomfort | 1 | (0.3) | 0 | | 0 | | 1 | (0.2) |
| Surgical and medical procedures | 1 | (0.3) | 0 | | 0 | | 1 | (0.2) |
| Dental local anaesthesia | 1 | (0.3) | 0 | | 0 | | 1 | (0.2) |
| Vascular disorders | 0 | | 0 | | 1 | (1.1) | 1 | (0.2) |
| Hypertension | 0 | | 0 | | 1 | (1.1) | 1 | (0.2) |
| Location: Ocular | | | | | | | | |
| Any TEAE | 26 | (7.4) | 10 | (11.2) | 9 | (10.2) | 45 | (8.5) |
| Eye disorders | 24 | (6.9) | 9 | (10.1) | 9 | (10.2) | 42 | (8.0) |
| Vision blurred | 7 | (2.0) | 5 | (5.6) | 4 | (4.45) | 16 | (3.0) |
| Foreign body sensation in eyes | 5 | (1.4) | 2 | (2.2) | 1 | (1.1) | 8 | (1.5) |
| Eye irritation | 2 | (0.6) | 2 | (2.2) | 2 | (2.3) | 6 | (1.1) |
| Eye pain | 2 | (0.6) | 2 | (2.2) | 2 | (2.3) | 6 | (1.1) |

| | | | | | | | |
|---|---|---|---|---|---|---|---|
| Halo vision | 1 | (0.3) | 1 | (1.1) | 0 | 2 | (0.4) |

| | | | | | | |
|---|---|---|---|---|---|---|
| Photophobia | 2 | (0.6) | 0 | 0 | 2 | (0.4) |
| Photopsia | 2 | (0.6) | 0 | 0 | 2 | (0.4) |
| Visual impairment | 2 | (0.6) | 0 | 0 | 2 | (0.4) |
| Abnormal sensation in eye | 1 | (0.3) | 0 | 0 | 1 | (0.2) |
| Corneal oedema | 1 | (0.3) | 0 | 0 | 1 | (0.2) |
| Diplopia | 1 | (0.3) | 0 | 0 | 1 | (0.2) |

| | | | | | | |
|---|---|---|---|---|---|---|
| Eye discharge | 0 | 1 | (1.1) | 0 | 1 | (0.2) |

| | | | | | | |
|---|---|---|---|---|---|---|
| Glare | 1 | (0.3) | 0 | 0 | 1 | (0.2) |

| | | | | | | |
|---|---|---|---|---|---|---|
| Lacrimation increased | 0 | 0 | 1 | (1.1) | 1 | (0.2) |

| | | | | | | |
|---|---|---|---|---|---|---|
| Ocular discomfort | 1 | (0.3) | 0 | 0 | 1 | (0.2) |

| | | | | | | |
|---|---|---|---|---|---|---|
| Periorbital discomfort | 0 | 1 | (1.1) | 0 | 1 | (0.2) |

| Visual field defect | 1 | (0.3) | 0 | 0 | 1 | (0.2) |
|---|---|---|---|---|---|---|
| General disorders and administration | 1 | (0.3) | 0 | 0 | 1 | (0.2) |
| site conditions | | | | | | |
| Installation site pain | 1 | (0.3) | 0 | 0 | 1 | (0.2) |
| Injury, poisoning and procedural | 2 | (0.6) | 0 | | | |
| complications | | | | | | |
| Corneal abrasion | 1 | (0.3) | 0 | | | |
| Procedural pain | 1 | (0.3) | 0 | | | |

| | | | | | | |
|---|---|---|---|---|---|---|
| Nervous system disorders | 0 | 1 | (1.1) | 0 | 1 | (0.2) |
| Nystagmus | 0 | 1 | (1.1) | 0 | 1 | (0.2) |

TABLE 59
Summary of Treatment-Related Treatment-Emergent Adverse Events
by System Organ Class and Preferred Term (Safety Analysis Set)

| | MELT-300 | Midazolam | Placebo | Overall |
|---|---|---|---|---|
| System Organ Class | (N = 350) | (N = 89) | (N = 88) | (N = 527) |
| Preferred Term | n(%) | n(%) | n( %) | n(%) |
| Any Treatment-Related TEAE | 25 (7.1) | 2 (2.2) | 4 (4.5) | 31 (5.9) |
| Cardiac disorders | 2 (0.6) | 0 | 0 | 2 (0.4) |
| Tachycardia | 1 (0.3) | 0 | 0 | 1 (0.2) |
| Bradycardia | 1 (0.3) | 0 | 0 | 1 (0.2) |
| Eye disorders | 1 (0.3) | 0 | 0 | 1 (0.2) |
| Visual impairment | 1 (0.3) | 0 | 0 | 1 (0.2) |
| Gastrointestinal disorders | 2 (0.6) | | | |
| Hypoaesthesia oral | 1 (0.3) | | | |
| Nausea | 1 (0.3) | | | |
| Injury, poisoning and procedural | 4 (1.1) | 0 | 0 | 4 (0.8) |
| complications | | | | |
| Procedural nausea | 4 (1.1) | 0 | 0 | 4 (0.8) |
| Procedural vomiting | 2 (0.6) | 0 | 0 | 2 (0.4) |
| Investigations | 6 (1.7) | 0 | 3 (3.4) | 9 (1.7) |
| Blood pressure increased | 3 (0.9) | 0 | 2 (2.3) | 5 (0.9) |
| Oxygen saturation decreased | 3 (0.9) | 0 | 1 (1.1) | 4 (0.8) |
| Nervous system disorders | 5 (1.4) | 2 (2.2) | 1 (1.1) | 8 (1.5) |
| Headache | 2 (0.6) | 1 (1.1) | 1 (1.1) | 4 (0.8) |
| Dizziness | 1 (0.3) | 0 | 0 | 1 (0.2) |
| Lethargy | 1 (0.3) | 0 | 0 | 1 (0.2) |
| Nystagmus | 0 | | 0 | 1 (0.2) |
| Paraesthesia | 0 | 1 (1.1) | 0 | 1 (0.2) |
| Taste disorder | 1 (0.3) | 1 (1.1) | 0 | 1 (0.2) |
| Product issues | 6 (1.7) | 0 | 0 | 6 (1.1) |
| Product after taste | 6 (1.7) | 0 | 0 | 6 (1.1) |
| Psychiatric disorders | 1 (0.3) | 0 | 0 | 1 (0.2) |
| Anxiety | 1 (0.3) | 0 | 0 | 1 (0.2) |
| Respiratory, thoracic, and mediastinal | 1 (0.3) | 1 (1.1) | 1 (1.1) | 3 (0.6) |
| disorders | | | | |
| Hypoxia | 1 (0.3) | 1 (1.1) | 0 | 2 (0.4) |
| Hiccups | 0 | 0 | 1 (1.1) | 1 (0.2) |
| Surgical and medical procedures | 1 (0.3) | 0 | 0 | 1 (0.2) |
| Dental local anaesthesia | 1 (0.3) | 0 | 0 | 1 (0.2) |

What is claimed is:

1. A method of inducing procedural sedation in a subject, the method comprising sublingually administering a pharmaceutical composition comprising midazolam and ketamine, wherein the administration achieves a level of sedation for procedural sedation that lasts for a time period of 45 minutes or less.

2. The method of claim 1, wherein the procedural sedation lasts for a time period of 30 minutes or less.

3. The method of claim 1, wherein the procedural sedation lasts for a time period of 15 minutes or less.

4. The method of claim 1, wherein the level of sedation achieved is measured via the Ramsay sedation scale.

5. The method of claim 1, wherein the level of sedation achieved is greater than achieved by administering midazolam alone or ketamine alone.

6. The method of claim 1, wherein the midazolam is present in the pharmaceutical composition in an amount from about 0.2 mass % to about 5.0 mass %.

7. The method of claim 1, wherein the ketamine is present in the pharmaceutical composition in an amount from about 1.0 mass % to about 10.0 mass %.

8. The method of claim 1, wherein the weight ratio of midazolam to ketamine in the pharmaceutical composition is about 1:5 to about 1:20.

9. The method of claim 8, wherein the weight ratio of midazolam to ketamine in the pharmaceutical composition is about 3:25.

10. The method of claim 1, wherein the pharmaceutical composition comprises about 3 mg of midazolam and about 25 mg of ketamine.

11. The method of claim 8, wherein the weight ratio of midazolam to ketamine in the pharmaceutical composition is about 3:50.

12. The method of claim 1, wherein the pharmaceutical composition comprises about 3 mg of midazolam and about 50 mg of ketamine.

13. The method of claim 1, wherein the pharmaceutical composition comprises a matrix former.

14. The method of claim 13, wherein the matrix former comprises mannitol, gelatin, or a combination thereof.

15. The method of claim 13, wherein the matrix former is present in the pharmaceutical composition in an amount from about 40% to about 95% by weight of the pharmaceutical composition.

16. The method of claim 13, wherein the matrix former is present in the pharmaceutical composition in an amount from about 80% to about 95% by weight of the pharmaceutical composition.

17. The method of claim 13, wherein the pharmaceutical composition is a freeze-dried composition.

18. The method of claim 1, wherein the pharmaceutical composition further comprises a sweetener.

19. The method of claim 1, wherein the pharmaceutical composition further comprises a flavoring agent.

20. The method of claim 1, wherein the sublingual administration results in a Cmax of midazolam in the subject that is at least about 10% lower, at least about 20% lower, at least about 30% lower, at least about 40% lower, at least about 50% lower, at least about 60% lower, at least about 70% lower, or at least about 80% lower than a Cmax of midazolam resulting from intravenous administration of an equivalent amount of midazolam.

21. The method of claim 1, wherein the sublingual administration results in a Cmax of midazolam in the subject of less than or equal to about 140 ng/mL.

22. The method of claim 1, wherein the sublingual administration results in a Cmax of 1-hydroxymidazolam in the subject that is at least about 25% greater, at least about 50% greater, at least about 100% greater, or at least about 200% greater than a Cmax of 1-hydroxymidazolam resulting from intravenous administration of an equal amount of midazolam.

23. The method of claim 1, wherein the sublingual administration results in a Cmax of 1-hydroxymidazolam in the subject that is greater than or equal to about 6 ng/ml.

24. The method of claim 1, wherein the sublingual administration results in a Cmax of ketamine in the subject that is at least about 10% lower, at least about 20% lower, at least about 30% lower, at least about 40% lower, at least about 50% lower, at least about 60% lower, at least about 70% lower, or at least about 80% lower than a Cmax achieved from intravenous administration of an equal amount of ketamine.

25. The method of claim 1, wherein the sublingual administration results in a Cmax of ketamine in the subject of less than or equal to about 275 ng/ml.

26. The method of claim 1, wherein the sublingual administration results in a Cmax of norketamine in the subject that is at least about 10% greater, at least about 25% greater, at least about 50% greater, at least about 100% greater, at least about 150% greater, at least about 200% greater, at least about 300% greater, or at least about 400% greater than a Cmax of norketamine achieved from intravenous administration of an equal amount of ketamine.

27. The method of claim 1, wherein the sublingual administration results in a Cmax of norketamine in the subject of greater than or equal to about 30 ng/ml.

28. A method of inducing procedural sedation in a subject, the method comprising:
sublingually administering to the subject a first dose of a pharmaceutical composition comprising midazolam and ketamine; and
sublingually administering to the subject a second dose of the pharmaceutical composition after the sublingual administration of the first dose,
wherein the procedural sedation lasts 45 minutes or less.

29. A solid pharmaceutical composition formulated for sublingual and or buccal administration comprising about 3 mg of midazolam and about 50 mg of ketamine.
